(19) United States
(12) Patent Application Publication
(10) Pub. No.: US 2026/0306664 A1
(43) Pub. Date: Oct. 8, 2026

(54) PROPHYLACTIC STEROID REGIMEN FOR RECOMBINANT ADENO-ASSOCIATED VIRAL VECTOR THERAPIES

(71) Applicants: AbbVie Inc., North Chicago, IL (US); REGENXBIO Inc., Rockville, MD (US)

(72) Inventors: Francisco Lopez, Ladera Ranch, CA (US); Amber Lewis, Newport Beach, CA (US); Quoc Ho, Irvine, CA (US); Michael Robinson, Huntington Beach, CA (US); Camille Beniga, Irvine, CA (US); Marither Chuidian, Irvine, CA (US); Masara Issa, Laguna Hills, CA (US); Sherri Van Everen, Rocklin, CA (US); Saba Kadlec, Wauwatosa, WI (US); Stephen Pakola, Irvington, NY (US); Anna Abolian, San Francisco, CA (US)

(21) Appl. No.: 19/638,616

(22) Filed: Apr. 3, 2026

Related U.S. Application Data
(60) Provisional application No. 63/783,034, filed on Apr. 3, 2025.
(60) Provisional application No. 63/858,287, filed on Aug. 5, 2025.
(60) Provisional application No. 63/945,382, filed on Dec. 19, 2025.

(51) Int. Cl. A61K 48/00; A61K 31/573; A61K 39/395; A61P 27/00

(52) U.S. Cl. CPC A61K 48/0033; A61K 31/573; A61K 39/3955; A61K 48/005; A61K 48/0075; A61K 48/0083; A61P 27/00

(57) ABSTRACT

Provided herein are methods of treating neovascular age-related macular degeneration (nAMD) and diabetic retinopathy (DR) with or without center involved-diabetic macular edema (CI-DME) in a subject in need thereof, wherein the methods comprise administering surabgene lomparvovec treatment and a steroid treatment about three days prior to administration of surabgene lomparvovec, or about six to seven days prior to administration of surabgene lomparvovec.

### 1. CROSS REFERENCE TO RELATED APPLICATIONS

This application claims priority to U.S. provisional application No. 63/783,034 filed Apr. 3, 2025; U.S. provisional application No. 63/858,287 filed Aug. 5, 2025; and U.S. provisional application No. 63/945,382 filed Dec. 19, 2025, each of which are incorporated by reference in their entireties herein.

### 2. FIELD

Methods of treating neovascular age-related macular degeneration (nAMD) and diabetic retinopathy (DR) with or without center involved-diabetic macular edema (CI-DME) in a subject in need thereof, wherein the methods comprise administering adeno-associated viral vector-based gene therapy treatment.

### 3. BACKGROUND

Surabgene lomparvovec (also known as RGX-314) is an investigational AAV gene therapy that is being developed as a potential treatment for neovascular age-related macular degeneration (nAMD) and diabetic retinopathy (DR). The genetic material contained in the surabgene lomparvovec vector is a recombinant DNA encoding a humanized monoclonal IgG 1 kappa Fab, which is designed to bind and inhibit human VEGF and is similar to the approved anti-VEGF agent ranibizumab (LUCENTIS®; Genentech). The proposed treatment with surabgene lomparvovec is a single treatment, via one administration to the eye, expected to provide continuous levels of anti-VEGF activity in the range where established efficacy has been demonstrated for ranibizumab, which has been approved in many countries for IVT injection treatment in patients with nAMD among other indications. Ranibizumab is also approved by the US FDA and European Medical Agency for IVT injection treatment in patients with DR. A long-term duration of efficacy following a single intervention will be a significant benefit to patients who currently must undergo monthly or otherwise frequent injections of ranibizumab. It is expected that surabgene lomparvovec will offer patients an alternative treatment to approved anti-VEGF therapies and potentially reduce the treatment burden of currently available therapies while maintaining patients' vision with a favorable risk/benefit profile.

### 4. SUMMARY

Provided herein are methods of treating a retinal disease associated with hVEGF, such as neovascular age-related macular degeneration (nAMD), diabetic retinopathy (DR), or diabetic macular edema (DME), in a subject in need thereof, wherein the methods comprise administering adeno-associated viral vector-based gene therapy treatment.

In particular, it was surprisingly found that the methods described herein (and in particular the described steroid regimens beginning 3-7 days prior to SCS administration of surabgene lomparvovec to patients results in an unexpected amount of mitigation of inflammation associated with surabgene lomparvovec when compared to the administration of surabgene lomparvovec concomitantly with (i.e., on the same day) beginning a steroid regimen where more inflammation was observed.

In some embodiments, the method is a method of treating nAMD, DR or DME in a subject in need thereof, wherein the method comprises administering surabgene lomparvovec treatment and a steroid treatment, wherein (A) the surabgene lomparvovec treatment comprises administering a therapeutically effective amount of surabgene lomparvovec to the suprachoroidal space of an eye of the subject; and (B) the steroid treatment comprises administering a therapeutically effective amount of a steroid to the eye of the subject about three days prior to administration of surabgene lomparvovec.

In some embodiments, the method is a method of treating nAMD, DR or DME in a subject in need thereof, wherein the method comprises administering surabgene lomparvovec treatment and a steroid treatment, wherein (A) the surabgene lomparvovec treatment comprises administering a therapeutically effective amount of surabgene lomparvovec to the suprachoroidal space of an eye of the subject; and (B) the steroid treatment comprises administering a therapeutically effective amount of a steroid to the eye of the subject about six to seven days prior to administration of the surabgene lomparvovec.

In some embodiments of the method of treating nAMD, DR or DME, the steroid treatment enables administration of surabgene lomparvovec at doses equal to or greater than about 1×10¹² GC/eye. In some embodiments of the method of treating nAMD, the steroid treatment enables administration of surabgene lomparvovec at doses equal to or greater than about 5×10¹¹ GC/eye.

In some embodiments of the method of treating nAMD, DR or DME, the method results in an efficacy result, a safety result, or both; wherein the efficacy result is selected from the group consisting of (1) the subject having a reduction in choroidal neovascularization (CNV) lesion size and/or leakage area relative to baseline, (2) the subject having a gain of visual acuity relative to baseline, (3) the subject not having a significant loss of visual acuity relative to baseline, (4) the subject having a reduction in central retinal thickness (CRT) relative to a baseline, or (5) the subject showing a reduction in a supplemental anti-VEGF injection annualized rate after the administration of the surabgene lomparvovec, wherein the subject receiving no more than 2, no more than 1, or no supplemental anti-VEGF injections after the administration of the surabgene lomparvovec; and wherein the safety result is mitigation of ocular inflammation of episcleritis, anterior chamber cell, vitritis, iridocyclitis, iritis, eye inflammation, anterior chamber flare, vitreal cells, keratic precipitates, and uveitis.

In some embodiments of the method of treating nAMD, DR or DME, the steroid administration continues for about 7 to 12 weeks, or for about 7 to 14 weeks, or for about 7 to 20 weeks. In some embodiments of the method of treating nAMD, the steroid administration continues for 7 weeks, 12 weeks, 14 weeks or 20 weeks in a tapering regimen.

In another embodiment, the method is a method of treating diabetic retinopathy (DR) with or without center involved-diabetic macular edema (CI-DME) in a subject in need thereof, wherein the method comprises administering surabgene lomparvovec treatment and a steroid treatment; wherein (A) the surabgene lomparvovec treatment comprises administering a therapeutically effective amount of surabgene lomparvovec to the suprachoroidal space of an eye of the subject; and (B) the steroid treatment comprises administering a therapeutically effective amount of a steroid to the eye of the subject about three days prior to administration of the surabgene lomparvovec.

In another embodiment, the method is a method of treating diabetic retinopathy (DR) with or without center involved-diabetic macular edema (CI-DME) in a subject in need thereof, wherein the method comprises administering surabgene lomparvovec treatment and a steroid treatment; wherein (A) the surabgene lomparvovec treatment comprises administering a therapeutically effective amount of surabgene lomparvovec to the suprachoroidal space of an eye of the subject; and (B) the steroid treatment comprises administering a therapeutically effective amount of a steroid to the eye of the subject about six to seven days prior to administration of the surabgene lomparvovec.

In some embodiments of the method of treating DR with or without CI-DME, the steroid treatment enables administration of surabgene lomparvovec at doses equal to or greater than about 1×10¹² GC/eye;

In some embodiments of the method of treating DR with or without CI-DME, the method results in an efficacy result, a safety result, or both; wherein the efficacy result is selected from the group consisting of (1) the subject maintaining best-corrected visual acuity (BCVA) relative to baseline, (2) the subject showing an improvement in their diabetic retinopathy severity scale (DRSS) score relative to baseline, (3) the subject no longer having CI-DME that was present when the surabgene lomparvovec was administered, (4) the subject not developing any sight-threatening ocular diabetic complications in the eye treated with surabgene lomparvovec, (5) the subject not developing ocular diabetic complications requiring treatment per standard of care (SOC) for such complications in the eye treated with surabgene lomparvovec, (6) if the subject develops neovascularization due to diabetic retinopathy requiring treatment per standard of care (SOC), then the subject only requires one SOC treatment to treat the neovascularization, (7) the subject not developing ocular diabetic complications requiring surgical intervention per standard of care (SOC) for such complications in the eye treated with surabgene lomparvovec, (8) the subject has a CST of less than 320 μm or no more than 290 μm after administration of the surabgene lomparvovec, (9) the subject not showing a worsening in their diabetic retinopathy severity scale (DRSS) score of two steps or more relative to baseline, (10) the subject not developing a vision-threatening complication (VTC) due to diabetic retinopathy, (11) subject having either a one-step reduction in their DRSS score, no change in their DRSS score, or a one-step improvement in their ETDRS DRSS score, (12) the subject maintains a ETDRS BCVA of within ±5 letters at week 48 after administration of the surabgene lomparvovec when compared to their ETDRS BCVA when the surabgene lomparvovec was administered, (13) the subject gains more than 5 letters of ETDRS BCVA at week 48 after administration of the surabgene lomparvovec when compared to their ETDRS BCVA when the surabgene lomparvovec was administered; and wherein the safety result is mitigation of ocular inflammation of episcleritis, anterior chamber cell, vitritis, iridocyclitis, iritis, eye inflammation, anterior chamber flare, vitreal cells, keratic precipitates, and uveitis.

In some embodiments of the method of treating DR with or without CI-DME, the steroid administration continues for about 7 to 12 weeks, or for about 7 to 14 weeks, or for about 7 to 16 weeks, or for about 7 to 20 weeks. In some embodiments of the method of treating DR with or without CI-DME, the steroid administration continues for 7 weeks, 12 weeks, 14 weeks, 16 weeks, or 20 weeks in a tapering regimen.

In another embodiment, the methods include a method of treating a retinal disease associated with hVEGF (e.g., nAMD, DR, or DME) in a subject in need thereof, the method comprising administering surabgene lomparvovec to the suprachoroidal space of an eye of said subject, wherein said administration of surabgene lomparvovec is at a dose in an amount of 1×10¹² GC/eye to 2×10¹² GC/eye; and administering difluprednate or a pharmaceutically acceptable salt thereof to the eye of said subject prior to and after administration of surabgene lomparvovec; wherein: the difluprednate or a pharmaceutically acceptable salt thereof is administered 1 drop 4 times daily for at least 3 days and up to 7 days prior to administration of the single dose of surabgene lomparvovec; and the dose of surabgene lomparvovec is safe and efficacious in treating said retinal disease at year 1 after administration of surabgene lomparvovec. For example, the difluprednate or pharmaceutically acceptable salt thereof is administered 1 drop 4 times daily for 7 days prior to administration of surabgene lomparvovec. In some embodiments, the dose of surabgene lomparvovec is safe and efficacious in treating said retinal disease at year 2 after administration of surabgene lomparvovec. In some embodiments, the difluprednate is topically administered.

In some embodiments of the method of treating a retinal disease associated with hVEGF, the difluprednate or pharmaceutically acceptable salt thereof is administered at a dose of 0.05% (w/v); for example, a 0.05% (w/v) ophthalmic emulsion of difluprednate.

In some embodiments of the method of treating a retinal disease associated with hVEGF, administration of the difluprednate or pharmaceutically acceptable salt thereof continues for 7 to 16 weeks or 7 to 20 weeks after said administration of surabgene lomparvovec.

In some embodiments of the method of treating a retinal disease associated with hVEGF, the method further comprises administering the difluprednate or a pharmaceutically acceptable salt thereof to the eye of said subject beginning Day 2 (the day immediately after the day of the administration of the dose of surabgene lomparvovec), 1 drop 4 times daily for 4 weeks, followed by 1 drop 3 times daily for 2 weeks, followed by 1 drop 2 times daily for 2 weeks, followed by 1 drop once daily for 2 weeks, and followed by 1 drop 1 time every other day for 2 weeks. In some embodiments, the method further comprises administering the difluprednate or a pharmaceutically acceptable salt thereof to the eye of said subject beginning Day 2 (the day immediately after the day of the administration of the dose of surabgene lomparvovec), 1 drop 4 times daily for 4 weeks, followed by 1 drop 3 times daily for 2 weeks, followed by 1 drop 2 times daily for 2 weeks, followed by 1 drop once daily for 2 weeks, and followed by 1 drop 1 time every other day for 2 weeks; wherein the dose of surabgene lomparvovec is in an amount of 1×10¹² GC/eye. In some embodiments, the method further includes administering 1 drop of the difluprednate or a pharmaceutically acceptable salt thereof to the eye of said subject during the day of, but after, the administration of the dose of surabgene lomparvovec.

In some embodiments of the method of treating a retinal disease associated with hVEGF, the method further comprises administering the difluprednate or a pharmaceutically acceptable salt thereof to the eye of said subject beginning Day 2 (the day immediately after the day of the administration of the dose of surabgene lomparvovec), 1 drop 4 times daily for 5 weeks, followed by 1 drop 3 times daily for 3 weeks, followed by 1 drop 2 times daily for 3 weeks, followed by 1 drop once daily for 3 weeks, and followed by 1 drop 1 time every other day for 2 weeks. In some embodiments, the method further comprises administering the difluprednate or a pharmaceutically acceptable salt thereof to the eye of said subject beginning Day 2 (the day immediately after the day of the administration of the dose of surabgene lomparvovec), 1 drop 4 times daily for 5 weeks, followed by 1 drop 3 times daily for 3 weeks, followed by 1 drop 2 times daily for 3 weeks, followed by 1 drop once daily for 3 weeks, and followed by 1 drop 1 time every other day for 2 weeks; wherein the dose of surabgene lomparvovec is in an amount of 1.5×10¹² GC/eye. In some embodiments, the method further includes administering 1 drop of the difluprednate or a pharmaceutically acceptable salt thereof to the eye of said subject during the day of, but after, the administration of the dose of surabgene lomparvovec.

In another embodiment, the methods include a method of treating a retinal disease associated with hVEGF (e.g., nAMD, DR, or DME) in a subject in need thereof, the method comprising, administering surabgene lomparvovec to the suprachoroidal space of an eye of said subject, wherein said administration of surabgene lomparvovec is at a dose in an amount of 1×10¹² GC/eye to 2×10¹² GC/eye; and administering prednisolone or a pharmaceutically acceptable salt thereof to the eye of said subject prior to and after administration of surabgene lomparvovec; wherein: the prednisolone or a pharmaceutically acceptable salt thereof is administered 2 drops 6 times daily for at least 3 days and up to 7 days prior to administration of surabgene lomparvovec; and the surabgene lomparvovec is safe and efficacious in treating said retinal disease at year 1 after administration of the single dose of surabgene lomparvovec. For example, the difluprednate or pharmaceutically acceptable salt thereof is administered 2 drops 6 times daily for 7 days prior to administration of surabgene lomparvovec. In some embodiments, the dose of surabgene lomparvovec is safe and efficacious in treating said retinal disease at year 2 after administration of surabgene lomparvovec.

In some embodiments of the method of treating a retinal disease associated with hVEGF, the prednisolone or pharmaceutically acceptable salt thereof is administered at a dose of 1% (w/v). In some embodiments, the prednisolone or pharmaceutically acceptable salt thereof is prednisolone acetate. In some embodiments, the prednisolone acetate is a prednisolone acetate ophthalmic solution.

In some embodiments of the method of treating a retinal disease associated with hVEGF, administration of the prednisolone or pharmaceutically acceptable salt thereof continues for 7 to 16 weeks or 7 to 20 weeks after said administration of surabgene lomparvovec.

In some embodiments of the method of treating a retinal disease associated with hVEGF, the method further comprising administering prednisolone or a pharmaceutically acceptable salt thereof to the eye of said subject beginning Day 2 (the day immediately after the day of the administration of the dose of surabgene lomparvovec), 2 drops 6 times daily for 4 weeks, followed by 2 drops 5 times daily for 1 week, followed by 2 drops 4 times daily for 1 week, followed by 2 drops 3 times daily for 1 week, followed by 2 drops 2 times daily for 1 week, followed by 2 drops once daily for 2 weeks, and followed by 2 drops 1 time every other day for 2 weeks. In some embodiments, the method further comprising administering prednisolone or a pharmaceutically acceptable salt thereof to the eye of said subject beginning Day 2 (the day immediately after the day of the administration of the dose of surabgene lomparvovec), 2 drops 6 times daily for 4 weeks, followed by 2 drops 5 times daily for 1 week, followed by 2 drops 4 times daily for 1 week, followed by 2 drops 3 times daily for 1 week, followed by 2 drops 2 times daily for 1 week, followed by 2 drops once daily for 2 weeks, and followed by 2 drops 1 time every other day for 2 weeks; wherein the dose of surabgene lomparvovec is in an amount of 1×10¹² GC/eye. In some embodiments, the method further includes administering 1 drop of the difluprednate or a pharmaceutically acceptable salt thereof to the eye of said subject during the day of, but after, the administration of the dose of surabgene lomparvovec.

In some embodiments of the method of treating a retinal disease associated with hVEGF, the method further comprises administering prednisolone or a pharmaceutically acceptable salt thereof to the eye of said subject beginning Day 2 (the day immediately after the day of the administration of the dose of surabgene lomparvovec), 2 drops 6 times daily for 5 weeks, followed by 2 drops 5 times daily for 2 weeks, followed by 2 drops 4 times daily for 2 weeks, followed by 2 drops 3 times daily for 2 weeks, followed by 2 drops 2 times daily for 2 weeks, followed by 2 drops once daily for 1 week, and followed by 2 drops 1 time every other day for 2 weeks. In some embodiments, the method further comprises administering prednisolone or a pharmaceutically acceptable salt thereof to the eye of said subject beginning Day 2 (the day immediately after the day of the administration of the dose of surabgene lomparvovec), 2 drops 6 times daily for 5 weeks, followed by 2 drops 5 times daily for 2 weeks, followed by 2 drops 4 times daily for 2 weeks, followed by 2 drops 3 times daily for 2 weeks, followed by 2 drops 2 times daily for 2 weeks, followed by 2 drops once daily for 1 week, and followed by 2 drops 1 time every other day for 2 weeks; wherein the dose of surabgene lomparvovec is in an amount of 1.5×10¹² GC/eye. In some embodiments, the method further includes administering 2 drops of the prednisolone or a pharmaceutically acceptable salt thereof to the eye of said subject during the day of, but after, the administration of the dose of surabgene lomparvovec. In some embodiments of the method, the method further comprises waiting up to 5 minutes, or from 5 to 10 minutes, or 5 minutes after administering a first drop of prednisolone or a pharmaceutically acceptable salt thereof to the eye of said subject to administer a second drop of prednisolone or a pharmaceutically acceptable salt thereof.

### 5. BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1 Schematic representation of surabgene lomparvovec vector genome.

FIG. 2 Study Schematic for Cohort 7 in Example 1. Abbreviations in the figure: D=Day; LTFU=long-term follow-up; PRN=as needed; Q4W-every 4 weeks; SD-OCT=spectral domain-optical coherence tomography; SV=Screening Visit; W=Week. DUREZOL or an equivalent steroid can be used.

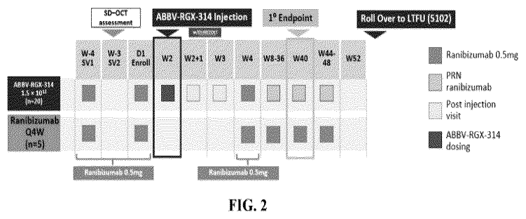

FIG. 3 Study Schematic for Part 2, Cohort A in Example 2. Abbreviations in the figure: CO=cross over; CRC=central reading center; D=Day; EOS=end of study; GC=genome copies; LTFU=long-term follow-up; OCT=optical coherence tomography; PRN=as needed; Q8W=every 8 weeks; SCS=suprachoroidal space; W=Week.

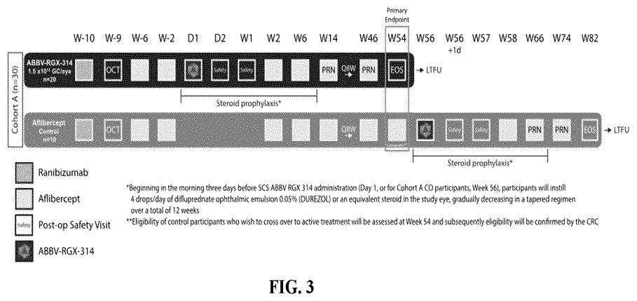

FIG. 4 Study Schematic for Example 3. Abbreviations and symbols in figure: D=Day; DRSS=diabetic retinopathy severity scale; EOS=end of study; Pt=part; SCS=suprachoroidal space; SV=Screening Visit; W=week; Y=year; asterisk=regimen will begin 3 days prior to Day 1 and will be tapered over 7 weeks.

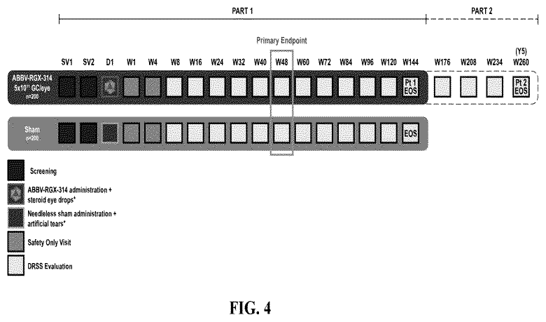

FIG. 5 provides concentration of Durezol 0.05% over time for Example 6.

FIG. 6 provides concentration of Pred Forte 1% (2 gtts) over time for Example 6.

FIG. 7 provides concentration of Pred Forte 1% (1 gtt) over time for Example 6.

### 6. DETAILED DESCRIPTION

Described herein are methods of treating neovascular age-related macular degeneration (nAMD), diabetic retinopathy (DR), or diabetic macular edema (DME) in a subject in need thereof, wherein the methods comprise administering surabgene lomparvovec and a steroid regimen.

Surabgene lomparvovec can be administered during a single visit by injection into the suprachoroidal space (SCS), which is the space between the sclera and the choroid), using a short, small-gauge needle (microneedle) that is designed to penetrate the sclera and reach the SCS. Delivery of surabgene lomparvovec to the SCS via microneedle can be performed in an outpatient clinical setting under sterile conditions, similar to IVT injections, thus eliminating the need for a surgical procedure and associated surgical risks with similar treatment effect.

However, despite the aforementioned advantages of SCS delivery, SCS delivery requires a larger dose than subretinal administration and can result in ocular inflammation (e.g., episcleritis). For example, U.S. Pat. No. 11,197,937, which is incorporated by reference in its entirety, granted Dec. 14. 2021, disclosed a higher immune response and other adverse effects at a surabgene lomparvovec dose of 1.0×10¹² GC/eye compared to lower subretinal doses. As a result, surabgene lomparvovec treatment using higher doses, such as doses of 1×10¹² GC/eye or greater has been considered problematic and preferably avoided.

It was surprisingly found that the methods described herein (and in particular the described steroid regimens beginning at least three days and up to seven days prior to SCS administration of the surabgene lomparvovec to patients) resulted in an unexpected amount of mitigation of inflammation and/or other adverse events associated with the surabgene lomparvovec when compared to the administration of surabgene lomparvovec concomitantly with (i.e., on the same day) beginning a steroid regimen where more inflammation and/or adverse events were observed. Orally administered steroids were not utilized, because based upon the inventors' experience, such steroids are unable to provide effective immune inhibition within the eye and thus are not comparable to the topical administration of steroids described herein. Without being bound by theory, such results may be unique to the eye due, at least in part, to the eye being a pseudo-immune privileged organ, and such a result may or may not apply to other organs.

Due to the surprising mitigation of inflammation achieved with the methods described herein, patients were able to receive higher doses of the SCS surabgene lomparvovec than what was previously achievable in patients who were administered surabgene lomparvovec with concurrent steroid administration. Thus, patients were able to achieve higher levels of efficacy with comparable safety profile than what was previously observed with lower doses, making it feasible to administer via SCS a single large dose of surabgene lomparvovec (e.g., 1×10¹² GC/eye, 1.5×10¹² GC/eye, or 2×10¹² GC/eye) whose therapeutic effect can last for one year, or even two years or longer, without additional surabgene lomparvovec dosing in the same eye.

### 6.1 Surabgene Lomparvovec

### 6.1.1 Structure and Description

Surabgene lomparvovec (also known as RGX-314, and AAV8.CB7.CI.amd42.RBG) has a CAS Registry Number of 2374935-18-7 and contain a vector genome the sequence of which is depicted as SEQ ID NO: 14 in U.S. Pat. No. 11,197,937, which is incorporated by reference in its entirety, granted Dec. 14. 2021.

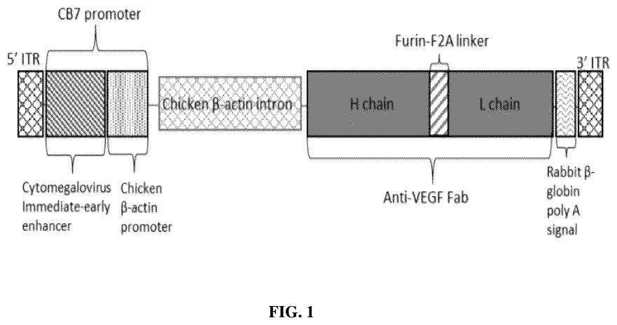

A schematic of the surabgene lomparvovec vector genome is shown in FIG. 1. The gene cassette is flanked by the AAV2 inverted terminal repeats and expresses human anti-VEGF Fab. Expression from the cassette is driven by a CB7 promoter, a hybrid of a CMV immediate-early enhancer and the chicken β-actin promoter. Transcription from this promoter is enhanced by the presence of the chicken β-actin intron. The polyadenylation signal for the expression cassette is from the rabbit β-globin gene. The nucleic acid sequences coding for the heavy and light chains of the ranibizumab Fab produced by surabgene lomparvovec are separated by a self-cleaving furin-F2A linker. The incorporation of the furin-F2A linker ensures expression of equal amounts of the heavy and the light chain polypeptides. Due to the mechanism of furin-mediated cleavage, surabgene lomparvovec contains an additional amino acid in the last position of the heavy chain in addition to all the amino acids normally found in ranibizumab.

Ranibizumab is a humanized anti-VEGF-A monoclonal antibody sold by Genentech under the brand name LUCENTIS®. The ranibizumab Fab fragment is comprised of one variable domain and one constant domain from both the heavy and light chains of the ranibizumab mAb.

Pharmaceutical compositions of “surabgene lomparvovec” include all pharmaceutically acceptable compositions comprising “surabgene lomparvovec” and one or more diluents, vehicles and/or excipients.

In certain embodiments of the methods provided herein, the method is a method of treating neovascular age-related macular degeneration (nAMD). In certain embodiments, the method is a method of treating diabetic retinopathy (DR). In certain embodiments, the method is a method of treating diabetic retinopathy (DR) with center involved-diabetic macular edema (CI-DME). In certain embodiments, the method is a method of treating diabetic retinopathy (DR) without center involved-diabetic macular edema (CI-DME). In certain embodiments, the method is a method of treating diabetic macular edema (DME).

In certain embodiments of the methods provided herein, the method results in the prevention of ocular inflammation. In certain embodiments of the methods provided herein, the method results in the prevention of intraocular inflammation. In certain embodiments of the methods provided herein, the method results in the prevention of episcleritis. In certain embodiments of the methods provided herein, the method results in the prevention of DR progression. In certain embodiments of the methods provided herein, the method results in the prevention of NPDR progression. In certain embodiments of the methods provided herein, the method results in the prevention of NPDR with CI-DME progression. In certain embodiments of the methods provided herein, the method results in the prevention of NPDR progression without CI-DME progression. In certain embodiments of the methods provided herein, the method results in the prevention of nAMD progression. In certain embodiments of the methods provided herein, the method results in the prevention of Vision Threatening Complications (VTCs).

In certain embodiments, the methods provided herein are for the administration to patients diagnosed with moderately-severe NPDR to severe NPDR without CI-DME. In certain embodiments, the methods provided herein are for the administration to patients diagnosed with DR with CI-DME

In certain embodiments, the patients have been shown to be responsive to treatment with an anti-VEGF antigen-binding fragment injected intravitreally prior to treatment with surabgene lomparvovec. In specific embodiments, the patients have previously been treated with LUCENTIS® (ranibizumab), EYLEA® (aflibercept), and/or AVASTIN® (bevacizumab), and have been found to be responsive to one or more of said LUCENTIS® (ranibizumab), EYLEA® (aflibercept), and/or AVASTIN® (bevacizumab).

In certain embodiments, surabgene lomparvovec is supplied as a frozen sterile, single use solution of the active ingredient in a formulation buffer.

Therapeutically effective doses of surabgene lomparvovec can be administered suprachoroidally (e.g., by suprachoroidal injection) in a volume of 100 μL or less, for example, in a volume of 50-100 μL. Suprachoroidal administration procedures involve administration of a drug to the suprachoroidal space of the eye, and are normally performed using a suprachoroidal drug delivery device such as a microinjector with a microneedle (see, e.g., Hariprasad, 2016, Retinal Physician 13:20-23; Goldstein, 2014, Retina Today 9 (5): 82-87; each of which is incorporated by reference herein in its entirety). The suprachoroidal drug delivery devices that can be used to deposit the expression vector in the suprachoroidal space according to the embodiments described herein include, but are not limited to, suprachoroidal drug delivery devices manufactured by Clearside® Biomedical, Inc. (see, for example, Hariprasad, 2016, Retinal Physician 13:20-23). Suprachoroidal administration should result in delivery of the soluble transgene product to the retina, the vitreous humor, and/or the aqueous humor. The expression of the transgene product (e.g., the encoded anti-VEGF antibody) by retinal cells, e.g., rod, cone, retinal pigment epithelial, horizontal, bipolar, amacrine, ganglion, and/or Müller cells, results in delivery and maintenance of the transgene product in the retina, the vitreous humor, and/or the aqueous humor. In a specific embodiment, doses that maintain a concentration of the transgene product at a Cₘᵢₙ of at least 0.330 μg/mL in the vitreous humour, or 0.110 μg/mL in the aqueous humour (the anterior chamber of the eye) for three months are desired; thereafter, vitreous Cₘᵢₙ concentrations of the transgene product ranging from 1.70 to 6.60 μg/mL, and/or aqueous Cₘᵢₙ concentrations ranging from 0.567 to 2.20 μg/mL should be maintained. However, because the transgene product is continuously produced, maintenance of lower concentrations can be effective. In a specific embodiment, the concentration of the transgene product can be measured in patient samples of the vitreous humour and/or aqueous from the anterior chamber of the treated eye. Alternatively, vitreous humour concentrations can be estimated and/or monitored by measuring the patient's serum concentrations of the transgene product—the ratio of systemic to vitreal exposure to the transgene product is about 1:90,000. (E.g., see, vitreous humor and serum concentrations of ranibizumab reported in Xu L, et al., 2013, Invest. Opthal. Vis. Sci. 54:1616-1624, at p. 1621 and Table 5 at p. 1623, which is incorporated by reference herein in its entirety).

Vector transgenes have the potential to spread to unintended recipients from shedding (release of vectors that did not infect the target cells and were cleared from the body via feces or bodily fluids), mobilization (transgene replication and transfer out of the target cell), or germ line transmission (genetic transmission to offspring through semen). Vector shedding may be determined for example by measuring vector DNA in biological fluids such as tears, serum or urine using quantitative polymerase chain reaction. In some embodiments, no vector gene copies are detectable in a biological fluid (e.g., tears, serum or urine) at any time point after administration of the vector. In some embodiments, less than 1000, less than 500, less than 100, less than 50 or less than 10 vector gene copies/5 μL are detectable by quantitative polymerase chain reaction in a biological fluid (e.g., tears, serum or urine) at any point after administration. In specific embodiments, 210 vector gene copies/5 μL or less are detectable in serum. In some embodiments, less than 1000, less than 500, less than 100, less than 50 or less than 10 vector gene copies/5 μL are detectable by quantitative polymerase chain reaction in a biological fluid (e.g., tears, serum or urine) by 1, 2, 3, 4, 5, 6, 7, 8, 9, 10, 11, 12, 13 or 14 weeks after administration. In specific embodiments, no vector gene copies are detectable in serum by week 14 after administration of the vector.

The goal of gene therapy treatment provided herein is to slow or arrest the progression of retinal degeneration, and to slow or prevent loss of vision with minimal intervention/invasive procedures. Efficacy may be monitored by measuring BCVA (Best-Corrected Visual Acuity), intraocular pressure, slit lamp biomicroscopy, indirect ophthalmoscopy, SD-OCT (Spectral Domain-Optical Coherence Tomography), electroretinography (ERG). Signs of vision loss, infection, inflammation and other safety events, including retinal detachment may also be monitored. Retinal thickness may be monitored to determine efficacy of the treatments provided herein. Without being bound by any particular theory, thickness of the retina may be used as a clinical readout, wherein the greater reduction in retinal thickness or the longer period of time before thickening of the retina, the more efficacious the treatment. Retinal thickness may be determined, for example, by SD-OCT. SD-OCT is a three-dimensional imaging technology which uses low-coherence interferometry to determine the echo time delay and magnitude of backscattered light reflected off an object of interest. OCT can be used to scan the layers of a tissue sample (e.g., the retina) with 3 to 15 μm axial resolution, and SD-OCT improves axial resolution and scan speed over previous forms of the technology (Schuman, 2008, Trans. Am. Opthamol. Soc. 106:426-458). Retinal function may be determined, for example, by ERG. ERG is a non-invasive electrophysiologic test of retinal function, approved by the FDA for use in humans, which examines the light sensitive cells of the eye (the rods and cones), and their connecting ganglion cells, in particular, their response to a flash stimulation.

### 6.1.2 Pharmaceutical Compositions

Compositions are described comprising surabgene lomparvovec and a suitable carrier. In certain embodiments of the methods provided herein, surabgene lomparvovec is formulated to be suitable for administration to the suprachoroidal space of the eye of the subject. In some embodiments, the formulated composition is a formulation and/or pharmaceutical composition described in WO2021/071835, which is incorporated herein by reference.

### 6.2 Methods of Treatment

Described herein are methods of treating neovascular age-related macular degeneration (nAMD), diabetic retinopathy (DR), or diabetic macular edema (DME) in a subject in need thereof, wherein the methods comprise administering surabgene lomparvovec and a steroid regimen.

In particular, it was surprisingly found that the methods described herein (and in particular the described steroid regimens beginning at least three days and up to seven days prior to SCS administration of the surabgene lomparvovec to patients) resulted in an unexpected amount of mitigation of inflammation and/or other adverse events associated with the surabgene lomparvovec when compared to the administration of surabgene lomparvovec concomitantly with (i.e., on the same day) beginning a steroid regimen where more inflammation was observed. Orally administered steroids were not utilized, because based upon the inventors' experience, such steroids are unable to provide effective immune inhibition within the eye and thus are not comparable to the topical administration of steroids described herein. Without being bound by theory, such results are believed to be unique to the eye due, at least in part, to the eye being a pseudo-immune privileged organ, and such a result may or may not apply to other organs.

Due to the surprising mitigation of inflammation achieved with the methods described herein, patients were able to receive higher doses of the surabgene lomparvovec than what was previously achievable in patients who were administered surabgene lomparvovec with concurrent steroid administration. Thus, patients were able to achieve higher levels of efficacy than what was previously observed with lower doses.

### 6.2.1 Method for the Delivery of Surabgene Lomparvovec

Methods are described for the administration of a therapeutically effective amount of surabgene lomparvovec to human subjects having an ocular disease, particularly an ocular disease caused by increased neovascularization. More particularly, methods are described for administration of a therapeutically effective amount of surabgene lomparvovec to patients having neovascular age-related macular degeneration (nAMD), diabetic retinopathy (DR), or diabetic macular edema (DME), in particular, for suprachoroidal administration are described.

Methods are described for suprachoroidal administration of a therapeutically effective amount of surabgene lomparvovec to patients diagnosed with neovascular age-related macular degeneration (nAMD) or diabetic retinopathy (DR) by suprachoroidal injection.

Also provided herein are methods for suprachoroidal administration of a therapeutically effective amount of surabgene lomparvovec via the suprachoroidal space.

In an embodiment provided herein is a method of suprachoroidal administration for treating neovascular age-related macular degeneration (nAMD) or diabetic retinopathy (DR), comprising administering to the suprachoroidal space in the eye of a human subject in need of treatment surabgene lomparvovec, such that the administration results in treatment of neovascular age-related macular degeneration or diabetic retinopathy. In certain embodiments, the administering step is by injecting the surabgene lomparvovec into the suprachoroidal space using a suprachoroidal drug delivery device. In certain embodiments, the suprachoroidal drug delivery device is a microinjector. In certain embodiments, the treatment requires one injection. In certain embodiments, the treatment requires two injections into the eye in need of treatment.

In certain embodiments, delivery to the suprachoroidal space can be performed using the methods and/or devices described and disclosed in International Publication Nos. WO 2016/042162, WO 2017/046358, WO 2017/158365, and WO 2017/158366, each of which is incorporated by reference in its entirety.

In certain embodiments, delivery of surabgene lomparvovec to the suprachoroidal space is performed using the investigational Clearside SCS Microinjector®. For each surabgene lomparvovec vial, 2 syringes are drawn up: one with a 900-μm needle and one with a 1100-μm needle. The needles are designed to penetrate the sclera and reach the SCS. The administration procedure starts with the 900-μm needle and the 1100-μm needle serves as a backup in the event a longer needle is needed. Administration is performed in the clinic under aseptic conditions.

In some embodiments, the suprachoroidal drug delivery device is a syringe with a 1 millimeter 30 gauge needle. In some embodiments, the syringe has a larger circumference (e.g., 29 gauge needle). During an injection using this device, the needle pierces to the base of the sclera and fluid containing drug enters the suprachoroidal space, leading to expansion of the suprachoroidal space. As a result, there is tactile and visual feedback during the injection. Following the injection, the fluid flows posteriorly and absorbs dominantly in the choroid and retina. This results in the production of transgene protein from all retinal cell layers and choroidal cells. Using this type of device and procedure allows for a quick and easy in-office procedure with low risk of complications.

In certain embodiments, surabgene lomparvovec is administered by multiple suprachoroidal injections. In certain embodiments, surabgene lomparvovec is administered by double suprachoroidal injections. In certain embodiments, the first injection in the right eye is administered in the superior temporal quadrant (i.e., between the 10 o'clock and 11 o'clock positions), and the second injection in the same eye is administered in the inferior nasal quadrant (i.e., between the 4 o'clock and 5 o'clock positions). In certain embodiments, the first injection in the right eye is administered in the inferior nasal quadrant (i.e., between the 4 o'clock and 5 o'clock positions), and the second injection in the same eye is administered in the superior temporal quadrant (i.e., between the 10 o'clock and 11 o'clock positions). In certain embodiments, the first injection in the left eye is administered in the superior temporal quadrant (i.e., between the 1 o'clock and 2 o'clock positions), and the second injection in the same eye is administered in the inferior nasal quadrant (i.e., between the 7 o'clock and 8 o'clock positions). In certain embodiments, the first injection in the left eye is administered in the inferior nasal quadrant (i.e., between the 7 o'clock and 8 o'clock positions), and the second injection in the same eye is administered in the superior temporal quadrant (i.e., between the 1 o'clock and 2 o'clock positions).

In certain embodiments, surabgene lomparvovec is administered by a single suprachoroidal injection. In certain embodiments, the single injection in the right eye is administered in the superior temporal quadrant (i.e., between the 10 o'clock and 11 o'clock positions). In certain embodiments, the single injection in the right eye is administered in the inferior nasal quadrant (i.e., between the 4 o'clock and 5 o'clock positions). In certain embodiments, the single injection in the left eye is administered in the superior temporal quadrant (i.e., between the 1 o'clock and 2 o'clock positions). In certain embodiments, the single injection in the left eye is administered in the inferior nasal quadrant (i.e., between the 7 o'clock and 8 o'clock positions).

### 6.2.2 Target Patient Populations

The subjects treated in accordance with the methods described herein can be any mammals such as rodents, domestic animals such as dogs or cats, or primates, e.g. non-human primates. In a one embodiment, the subject is a human. In certain embodiments, the methods provided herein are for the administration to patients diagnosed with neovascular age-related macular degeneration (nAMD) or diabetic retinopathy (DR).

In certain embodiments, the methods provided herein are for the administration to patients diagnosed with severe AMD. In certain embodiments, the methods provided herein are for the administration to patients diagnosed with attenuated AMD.

In certain embodiments, the methods provided herein are for the administration to patients diagnosed with severe neovascular (or “wet”) AMD. In certain embodiments, the methods provided herein are for the administration to patients diagnosed with attenuated neovascular AMD.

In certain embodiments, the methods provided herein are for the administration to patients diagnosed with AMD who have been identified as responsive to treatment with an anti-VEGF antibody.

In certain embodiments, the methods provided herein are for the administration to patients diagnosed with AMD who have been identified as responsive to treatment with an anti-VEGF antigen-binding fragment.

In certain embodiments, the methods provided herein are for the administration to patients diagnosed with AMD who have been identified as responsive to treatment with an anti-VEGF antigen-binding fragment injected intravitreally prior to treatment with surabgene lomparvovec.

In certain embodiments, the methods provided herein are for the administration to patients diagnosed with AMD who have been identified as responsive to treatment with LUCENTIS® (ranibizumab), EYLEA® (aflibercept), and/or AVASTIN® (bevacizumab).

In certain embodiments, a patient diagnosed with AMD is identified as responsive to treatment with an anti-VEGF antigen-binding fragment (e.g., ranibizumab) if the patient has improvement in fluid after intravitreal injection of the anti-VEGF antigen-binding fragment to the patient prior to treatment with gene therapy. In certain embodiments, a patient diagnosed with AMD is identified as responsive to treatment with an anti-VEGF antigen-binding fragment (e.g., ranibizumab) if the patient has improvement in fluid and has a central retinal thickness (CRT)<400 μm after intravitreal injection of the anti-VEGF antigen-binding fragment to the patient prior to treatment with gene therapy. In some embodiments, the anti-VEGF antigen-binding fragment is intravitreally injected to the patient at 0.5 mg per month for two months prior to treatment with gene therapy. In other embodiments, the anti-VEGF antigen-binding fragment is intravitreally injected to the patient at 0.5 mg per month for three months prior to treatment with gene therapy. In another embodiment, a patient has improvement in fluid if he or she has an improvement in inner retinal (parafovea 3 mm) fluid of >50 μm or 30% relative to the level prior to the intravitreal injection of the anti-VEGF antigen-binding fragment, or has an improvement in center subfield thickness of >50 μm or 30% as determined by the CRC relative to the level prior to the intravitreal injection of the anti-VEGF antigen-binding fragment.

In certain embodiments, the methods provided herein are for the administration to patients diagnosed with AMD who have disease other than fluid contributing to an increase in CRT (i.e., pigment epithelial detachment (PED) or subretinal hyperreflective material (SHRM)) and who have <75 μm of fluid (intraretinal or subretinal), as determined by the CRC.

In certain embodiments of the methods described herein, the patient has a BCVA in the eye to be treated that is ≤20/20 and ≥20/400 before treatment. In certain embodiments, the patient has a BCVA in the eye to be treated that is ≤20/25 and ≥20/125 (≤83 and ≥44 ETDRS letters) before treatment. In a specific embodiment, the patient has a BCVA in the eye to be treated that is ≤20/63 and ≥20/400 before treatment. In certain embodiments, the patient has a BCVA in the eye to be treated that is ≥69 ETDRS letters (≥20/40 Snellen).

In certain embodiments of the methods described herein, the patient has a negative or low serum titer result (≤300) for recombinant viral vector neutralizing antibodies (NAbs). In certain embodiments, the patients has a negative or low serum titer result (≤300) for AAV8 NAbs. In other embodiments, the patients has a serum titer result >300 for recombinant viral vector NAbs. In certain embodiments, the patients has a serum titer result >300 for AAV8 NAbs. In certain embodiments, the methods described herein are effective for treatment of AMD or DR in patients having a negative or low serum titer result (≤300) for AAV8 NAbs or in patients having a serum titer result >300 for recombinant viral vector NAbs. In certain embodiments, the methods described herein are as effective for treatment of AMD and DR in patients having a negative or low serum titer result (≤300) for AAV8 NAbs as in patients having a serum titer result >300 for recombinant viral vector NAbs.

In certain embodiments of the methods described herein, the patient is not concurrently having an anticoagulation therapy.

In certain embodiments, the methods provided herein are for the administration to patients diagnosed with severe diabetic retinopathy. In certain embodiments, the methods provided herein are for the administration to patients diagnosed with attenuated diabetic retinopathy.

Diabetic retinopathy is graded according to whether it is non-proliferative diabetic retinopathy (NPDR) or the more advanced proliferative diabetic retinopathy (PDR), and then with regards to the severity of the NPDR or PDR.

One method of grading diabetic retinopathy that is common in clinical studies is the Early Treatment Diabetic Retinopathy Study Diabetic Retinopathy Severity Scale, or ETDRS-DRSS (also referred to simply as DRSS score).

The DRSS scores are a series of numbers that in ascending severity which classify the diabetic retinopathy severity based on various anatomical finding. On that scale, NPDR corresponds to levels 35 (mild NPDR), 43 (moderate NPDR), 47 (moderately severe NPDR, and 53 (severe NPDR), while PDR corresponds to levels 61 (mild PDR), 65 (moderate PDR), 71 (high risk PDR), 75 (high risk PDR), 81 (advanced PDR), and 85 (advanced PDR). See, Early Treatment Diabetic Retinopathy Study Research Group. “Fundus photographic risk factors for progression of diabetic retinopathy: ETDRS report number 12.” Ophthalmology 98.5 (1991): 823-833. See also, Staurenghi, Giovanni, et al. “Impact of baseline Diabetic Retinopathy Severity Scale scores on visual outcomes in the VIVID-DME and VISTA-DME studies.” British Journal of Ophthalmology 102.7 (2018): 954-958.

In clinical practice, many ophthalmologists and retina specialists simply classify diabetic retinopathy by whether it is NPDR or PDR, and then divide the NPDR into three sub-levels: mild, moderate, and severe without generally using the particular DRSS scores (see, Koetting, C. “The four stages of diabetic retinopathy.” Modern Optometry (2019): 21-24). These classifications can be further subdivided by whether or not diabetic macular edema-(DME) is present, as is done with the ICD-10 codes for diabetic retinopathy which classifies diabetic retinopathy by the type of diabetes, the DR severity, and presence of DME (see, Dugan, Joy, and Jay Shubrook. “International classification of diseases, 10th revision, coding for diabetes.” Clinical Diabetes 35.4 (2017): 232-238).

In certain embodiments, the methods provided herein are for the administration to patients diagnosed with moderately-severe NPDR. In certain embodiments, the methods provided herein are for the administration to patients diagnosed with severe NPDR. In certain embodiments, the methods provided herein are for the administration to patients diagnosed with mild PDR. In certain embodiments, the methods provided herein are for the administration to patients diagnosed with moderate PDR. In certain embodiments, the methods provided herein are for the administration to patients diagnosed with moderately-severe NPDR to severe NPDR without CI-DME. In certain embodiments, the methods provided herein are for the administration to patients diagnosed with DR with CI-DME.

In certain embodiments, the methods provided herein are for the administration to patients whose ETDRS-DRSS Levels are 47, 53, 61 or 65. In certain embodiments, the methods provided herein are for the administration to patients whose ETDRS-DRSS Levels are Level 47. In certain embodiments, the methods provided herein are for the administration to patients whose ETDRS-DRSS Levels are Level 53. In certain embodiments, the methods provided herein are for the administration to patients whose ETDRS-DRSS Levels are Level 61. In certain embodiments, the methods provided herein are for the administration to patients whose ETDRS-DRSS Levels are Level 65. In certain embodiments, the methods provided herein are for the administration to patients whose BCVA ≥69 ETDRS letters (≥ 20/40 Snellen). In certain embodiments, the methods provided herein are for the administration to patients whose BCVA is between ≤20/25 and ≥20/125 (≤83 and ≥44 ETDRS letters). In certain embodiments, the methods provided herein are for the administration to patients whose BCVA in the study eye is 78 to 25 ETDRS letters (approximate Snellen equivalent 20/32 to 20/320).

In certain embodiments of the methods described herein, the patient has an Early Treatment Diabetic Retinopathy Study (ETDRS) BCVA letter score between ≤78 and ≥25 in the eye to be treated before treatment.

In certain embodiments, the subject treated in accordance with the methods described herein is female. In certain embodiments, the subject treated in accordance with the methods described herein is male. In certain embodiments, the subject treated in accordance with the methods described herein can be of any age. In certain embodiments, the subject treated in accordance with the methods described herein is 18 years old or older. In certain embodiments, the subject treated in accordance with the methods described herein is between 18-89 years of age. In certain embodiments, the subject treated in accordance with the methods described herein is between 25-89 years of age. In certain embodiments, the subject treated in accordance with the methods described herein has DR secondary to diabetes mellitus Type 1. In certain embodiments, the subject treated in accordance with the methods described herein has DR secondary to diabetes mellitus Type 2. In certain embodiments, the subject treated in accordance with the methods described herein is 18 years old or older with DR secondary to diabetes mellitus Type 1 or Type 2. In certain embodiments, the subject treated in accordance with the methods described herein is between 18-89 years of age with DR secondary to diabetes mellitus Type 1 or Type 2.

In certain embodiments, the subject treated in accordance with the methods described herein is phakic. In other specific embodiments, the subject treated in accordance with the methods described herein is pseudophakic.

In certain embodiments, the subject treated in accordance with the methods described herein has a hemoglobin A1c≤12%.

In certain embodiments, the subject treated in accordance with the methods described herein has best-corrected visual acuity (BCVA) in the eye to be treated of ≥69 ETDRS letters (approximate Snellen equivalent 20/40 or better).

In some embodiments, the method further comprises obtaining or having obtained a biological sample from the subject and determining that the subject has a serum level of hemoglobin A1c of less than or equal to 10%. In some embodiments, the method further comprises obtaining or having obtained a biological sample from the subject and determining that the subject has a serum level of hemoglobin A1c of less than or equal to 12%.

In some embodiments, the method prevents progression to proliferative stages of retinopathy in the subject.

In certain embodiments of the methods described herein, the patient has a negative or low serum titer result (≤300) for recombinant viral vector neutralizing antibodies (NAbs). In certain embodiments, the patient has a negative or low serum titer result (≤300) for AAV8 NAbs. In other embodiments, the patient has a serum titer result >300 for recombinant viral vector NAbs. In certain embodiments, the patient has a serum titer result >300 for AAV8 NAbs.

ETDRS-DR severity scale (DRSS) Levels are determined using standard 4-widefield or modified 7-field digital stereoscopic fundus photographs or equivalent; they may also be measured by monoscopic or stereo photography in accordance with Li et al., 2010, Retina Invest Ophthalmol Vis Sci. 2010; 51:3184-3192, or an analogous method.

### 6.2.3 Dosage and Mode of Administration

Therapeutically effective doses of surabgene lomparvovec according to the methods described herein can be administered suprachoroidally (e.g., by suprachoroidal injection) in a volume of 100 μL or less, for example, in a volume of 50-100 μL. The two injections may be administered during the same visit.

In certain embodiments, a therapeutically effective dose of surabgene lomparvovec is administered to the subject as a single dosage form. In certain embodiments, the therapeutically effective dose of surabgene lomparvovec is administered to the subject as a single injection. In certain embodiments, the therapeutically effective dose of surabgene lomparvovec is administered to the subject as two injections. In certain embodiments, the therapeutically effective dose of surabgene lomparvovec is administered to the subject as a single injection per eye. In certain embodiments, the therapeutically effective dose of surabgene lomparvovec is administered to the subject as two injections per eye.

In certain embodiments, surabgene lomparvovec is administered suprachoroidally (e.g., by suprachoroidal injection). In a specific embodiment, suprachoroidal administration (e.g., an injection into the suprachoroidal space) is performed using a suprachoroidal drug delivery device. Suprachoroidal drug delivery devices are often used in suprachoroidal administration procedures, which involve administration of a drug to the suprachoroidal space of the eye (see, e.g., Hariprasad, 2016, Retinal Physician 13:20-23; Goldstein, 2014, Retina Today 9 (5): 82-87; Baldassarre et al., 2017; each of which is incorporated by reference herein in its entirety). The suprachoroidal drug delivery devices that can be used to deposit the expression vector in the subretinal space according to the embodiments described herein include, but are not limited to, suprachoroidal drug delivery devices manufactured by Clearside® Biomedical, Inc. (see, for example, Hariprasad, 2016, Retinal Physician 13:20-23) and MedOne suprachoroidal catheters.

In a specific embodiment, the suprachoroidal drug delivery device is a syringe with a 30 G×900 μm needle, or a 30 G×1100 μm needle. In a specific embodiment, the suprachoroidal drug delivery device is a syringe with a 30 G×1100 μm needle. During an injection using this device, the needle pierces to the base of the sclera and fluid containing drug enters the suprachoroidal space, leading to expansion of the suprachoroidal space. As a result, there is tactile and visual feedback during the injection. Following the injection, the fluid flows posteriorly and absorbs dominantly in the choroid and retina. This results in the production of transgene protein from all retinal cell layers and choroidal cells. Using this type of device and procedure allows for a quick and easy in-office procedure with low risk of complications.

In certain embodiments of the methods described herein, surabgene lomparvovec is administered suprachoroidally (e.g., by suprachoroidal injection). In a specific embodiment, suprachoroidal administration (e.g., an injection into the suprachoroidal space) is performed using a suprachoroidal drug delivery device. Suprachoroidal drug delivery devices are often used in suprachoroidal administration procedures, which involve administration of a drug to the suprachoroidal space of the eye (see, e.g., Hariprasad, 2016, Retinal Physician 13:20-23; Goldstein, 2014, Retina Today 9 (5): 82-87; Baldassarre et al., 2017; each of which is incorporated by reference herein in its entirety). The suprachoroidal drug delivery devices that can be used to deposit the recombinant vector in the suprachoroidal space according to the embodiments described herein include, but are not limited to, suprachoroidal drug delivery devices manufactured by Clearside® Biomedical, Inc. (see, for example, Hariprasad, 2016, Retinal Physician 13:20-23) and MedOne suprachoroidal catheters. In another embodiment, the suprachoroidal drug delivery device that can be used in accordance with the methods described herein comprises the micro volume injector delivery system, which is manufactured by Altaviz (see FIGS. 8A and 8B) (see, e.g. International Patent Application Publication No. WO 2013/177215, United States Patent Application Publication No. 2019/0175825, and United States Patent Application Publication No. 2019/0167906) that can be used for any administration route described herein for eye administration. The micro volume injector delivery system may include a gas-powered module providing high force delivery and improved precision, as described in United States Patent Application Publication No. 2019/0175825 and United States Patent Application Publication No. 2019/0167906. In addition, the micro volume injector delivery system may include a hydraulic drive for providing a consistent dose rate, and a low-force activation lever for controlling the gas-powered module and, in turn, the fluid delivery. The micro volume injector is a micro volume injector with dose guidance and can be used with, for example, a suprachoroidal needle (for example, the Clearside® needle) or a subretinal needle. The benefits of using micro volume injector include: (a) more controlled delivery (for example, due to having precision injection flow rate control and dose guidance), (b) single surgeon, single hand, one finger operation; (c) pneumatic drive with 10 μL increment dosage; (d) divorced from the vitrectomy machine; (e) 400 μL syringe dose; (f) digitally guided delivery; (g) digitally recorded delivery; and (h) agnostic tip (for example, the MedOne 38 g needle and the Dorc 41 g needle can be used for subretinal delivery, while the Clearside® needle and the Visionisti OY adaptor can be used for suprachoroidal delivery). In another embodiment, the suprachoroidal drug delivery device that can be used in accordance with the methods described herein is a tool that comprises a normal length hypodermic needle with an adaptor (and also a needle guide) manufactured by Visionisti OY, which adaptor turns the normal length hypodermic needle into a suprachoroidal needle by controlling the length of the needle tip exposing from the adapter (see, for example, U.S. Design Pat. No. D878,575; and International Patent Application. Publication No. WO/2016/083669). In a specific embodiment, the suprachoroidal drug delivery device is a syringe with a 1 millimeter 30 gauge needle. During an injection using this device, the needle pierces to the base of the sclera and fluid containing drug enters the suprachoroidal space, leading to expansion of the suprachoroidal space. As a result, there is tactile and visual feedback during the injection. Following the injection, the fluid flows posteriorly and absorbs dominantly in the choroid and retina. This results in the production of therapeutic product from all retinal cell layers and choroidal cells. Using this type of device and procedure allows for a quick and easy in-office procedure with low risk of complications.

In certain embodiments, dosages are measured by genome copies per mL or the number of genome copies administered to the eye of the patient administered suprachoroidally, by suprachoroidal injection. In certain embodiments, 5×10¹¹ genome copies per mL to 2×10¹² genome copies per mL are administered. In another specific embodiment, 5×10¹¹ genome copies per mL to 1×10¹² genome copies per mL are administered. In another specific embodiment, 1×10¹² genome copies per mL to 1.5×10¹² genome copies per mL are administered. In another specific embodiment, 1.5×10¹² genome copies per mL to 2×10¹² genome copies per mL are administered. In another specific embodiment, 5×10¹¹ genome copies per mL are administered. In another specific embodiment, 1×10¹² genome copies per mL are administered. In another specific embodiment, 1.5×10¹² genome copies per mL are administered. In another specific embodiment, 2×10¹² genome copies per mL are administered. In another specific embodiment, 5×10¹² genome copies per mL to 2×10¹³ genome copies per mL are administered. In another specific embodiment, 5×10¹² genome copies per mL are administered. In another specific embodiment, 1×10¹³ genome copies per mL are administered. In another specific embodiment, 1.5×10¹³ genome copies per mL are administered. In another specific embodiment, 2×10¹³ genome copies per mL are administered.

In certain embodiments, 5×10¹¹ genome copies per eye are administered by suprachoroidal injection. In certain embodiments, 1×10¹² genome copies per eye are administered by suprachoroidal injection. In certain embodiments, 1.5×10¹² genome copies per eye are administered by suprachoroidal injection. In certain embodiments, 2×10¹² genome copies per eye are administered by suprachoroidal injection.

In certain embodiments, 5×10¹¹ genome copies per eye are administered by a single suprachoroidal injection. In certain embodiments, 1×10¹² genome copies per eye are administered by a single suprachoroidal injection. In certain embodiments, 1.5×10¹² genome copies per eye are administered by a single suprachoroidal injection. In certain embodiments, 2×10¹² genome copies per eye are administered by a single suprachoroidal injection.

In certain embodiments, 5×10¹¹ genome copies per eye are administered by two suprachoroidal injections. In certain embodiments, 1×10¹² genome copies per eye are administered by two suprachoroidal injections. In certain embodiments, 1.5×10¹² genome copies per eye are administered by two suprachoroidal injections. In certain embodiments, 2×10¹² genome copies per eye are administered by two suprachoroidal injections.

In certain embodiments, 5×10¹¹ genome copies per eye are administered by a single suprachoroidal injection in a volume of 100 μL. In certain embodiments, 1×10¹² genome copies per eye are administered by a single suprachoroidal injection in a volume of 100 μL. In certain embodiments, 1.5×10¹² genome copies per eye are administered by a single suprachoroidal injection in a volume of 100 μL. In certain embodiments, 2×10¹² genome copies per eye are administered by a single suprachoroidal injection in a volume of 100 μL.

In certain embodiments, 5×10¹¹ genome copies per eye are administered by two suprachoroidal injections in a volume of 50 μL each. In certain embodiments, 1×10¹² genome copies per eye are administered by two suprachoroidal injections in a volume of 50 μL each. In certain embodiments, 1.5×10¹² genome copies per eye are administered by two suprachoroidal injections in a volume of 50 μL each. In certain embodiments, 2×10¹² genome copies per eye are administered by two suprachoroidal injections in a volume of 50 μL each.

In certain embodiments, 5×10¹¹ genome copies per administration, or per eye are administered by suprachoroidal injection. In certain embodiments, 1×10¹² genome copies per administration, or per eye are administered by suprachoroidal injection. In certain embodiments, 1.5×10¹² genome copies per administration, or per eye are administered by suprachoroidal injection. In certain embodiments, 2×10¹² genome copies per eye are administered by a single suprachoroidal injection.

In certain embodiments, 5×10¹¹ genome copies per eye are administered by a single suprachoroidal injection. In certain embodiments, 1×10¹² genome copies per eye are administered by a single suprachoroidal injection in a volume of 100 μL. In certain embodiments, 1.5×10¹² genome copies per eye are administered by a single suprachoroidal injection in a volume of 100 μL. In certain embodiments, 2×10¹² genome copies per eye are administered by a single suprachoroidal injection in a volume of 100 μL.

In some embodiments described herein, the surabgene lomparvovec can be administered with the methods described herein to one eye of a subject. In other embodiments, the surabgene lomparvovec can be administered with the methods described herein to both eyes of a subject.

As used herein and unless otherwise specified, the term “about” means within plus or minus 10% of a given value or range.

The term “between” as used in a phrase as such “between A and B” or “between A-B” refers to a range including both A and B.

In certain embodiments, an infrared thermal camera can be used to detect changes in the thermal profile of the ocular surface after the administering of a solution which is cooler than body temperature to detect changes in the thermal profile of the ocular surface that allows for visualization of the spread of the solution, e.g., within the SCS, and can potentially determine whether the administration was successfully completed. This is because in certain embodiments the formulation containing the recombinant vector to be administered is initially frozen, brought to room temperature (68-72° F.), and thawed for a short period of time (e.g., at least 30 minutes) before administration, and thus the formulation is colder than the human eye (about 92° F.) (and sometimes even colder than room temperature) at the time of injection. The drug product is typically used within 4 hours of thaw and the warmest the solution would be is room temperature. In one embodiment, the procedure is videoed with infrared video.

Infrared thermal cameras can detect small changes in temperature. They capture infrared energy through a lens and convert the energy into an electronic signal. The infrared light is focused onto an infrared sensor array which converts the energy into a thermal image. The infrared thermal camera can be used for any method of administration to the eye, including any administration route described herein, for example, suprachoroidal administration. In a specific embodiment, the infrared thermal camera is an FLIR T530 infrared thermal camera. The FLIR T530 infrared thermal camera can capture slight temperature differences with an accuracy of +3.6° F. The camera has an infrared resolution of 76,800 pixels. The camera also utilizes a 24° lens capturing a smaller field of view. A smaller field of view in combination with a high infrared resolution contributes to more detailed thermal profiles of what the operator is imaging. However, other infrared camera can be used that have different abilities and accuracy for capturing slight temperature changes, with different infrared resolutions, and/or with different degrees of lens.

In a specific embodiment, the infrared thermal camera is an FLIR T420 infrared thermal camera. In a specific embodiment, the infrared thermal camera is an FLIR T440 infrared thermal camera. In a specific embodiment, the infrared thermal camera is an Fluke Ti400 infrared thermal camera. In a specific embodiment, the infrared thermal camera is an FLIRE60 infrared thermal camera. In a specific embodiment, the infrared resolution of the infrared thermal camera is equal to or greater than 75,000 pixels. In a specific embodiment, the thermal sensitivity of the infrared thermal camera is equal to or smaller than 0.05° C. at 30° C. In a specific embodiment, the field of view (FOV) of the infrared thermal camera is equal to or lower than 25°×25°.

In certain embodiments, an iron filer is used with the infrared thermal camera to detect changes in the thermal profile of the ocular surface. In one embodiment, the use of an iron filter is able to a generate pseudo-color image, wherein the warmest or high temperature parts are colored white, intermediate temperatures are reds and yellows, and the coolest or low temperature parts are black. In certain embodiments, other types of filters can also be used to generate pseudo-color images of the thermal profile.

The thermal profile for each administration method can be different. For example, in one embodiment, a successful suprachoroidal injection can be characterized by: (a) a slow, wide radial spread of the dark color, (b) very dark color at the beginning, and (c) a gradual change of injectate to lighter color, i.e., a temperature gradient noted by a lighter color. In one embodiment, an unsuccessful suprachoroidal injection can be characterized by: (a) no spread of the dark color, and (b) a minor change in color localized to the injection site without any distribution. In certain embodiments, the small localized temperature drop is result from cannula (low temperature) touching the ocular tissues (high temperature).

### 6.2.4 Sampling and Monitoring of Efficacy

Effects of the methods of treatment provided herein on visual deficits may be measured by BCVA (Best-Corrected Visual Acuity), intraocular pressure, slit lamp biomicroscopy, and/or indirect ophthalmoscopy. Extraocular movement may also be assessed. The intraocular pressure measurements may be conducted using Tonopen, iCare tonometer, or Goldmann applanation tonometry. The slit lamp examination may include an evaluation of the lids/lashes, conjunctiva/sclera, cornea, anterior chamber, iris, lens, and/or vitreous body.

In specific embodiments, effects of the methods provided herein on visual deficits may be measured by whether the human patient's eye that is treated by a method described herein achieves BCVA of greater than 43 letters post-treatment (e.g., 46-50 weeks or 98-102 weeks post-treatment). A BCVA of 43 letters corresponds to 20/160 approximate Snellen equivalent. In a specific embodiment, the human patient's eye that is treated by a method described herein achieves BCVA of greater than 43 letters post-treatment (e.g., 46-50 weeks or 98-102 weeks post-treatment).

In specific embodiments, effects of the methods provided herein on visual deficits may be measured by whether the human patient's eye that is treated by a method described herein achieves BCVA of greater than 84 letters post-treatment (e.g., 46-50 weeks or 98-102 weeks post-treatment). A BCVA of 84 letters corresponds to 20/20 approximate Snellen equivalent. In a specific embodiment, the human patient's eye that is treated by a method described herein achieves BCVA of greater than 84 letters post-treatment (e.g., 46-50 weeks or 98-102 weeks post-treatment). The BCVA testing may be conducted at a distance of 4 meters using ETDRS charts. For participants with reduced vision (inability to read ≥20 letters correctly at 4 meters), the BCVA testing may be conducted at a distance of 1 meter.

Effects of the methods of treatment provided herein on physical changes to eye/retina may be measured by SD-OCT (Spectral Domain-Optical Coherence Tomography).

Efficacy may be monitored as measured by electroretinography (ERG).

Effects of the methods of treatment provided herein may be monitored by measuring signs of vision loss, infection, inflammation and other safety events, including retinal detachment.

Retinal thickness may be monitored to determine efficacy of the treatments provided herein. Without being bound by any particular theory, thickness of the retina may be used as a clinical readout, wherein the greater reduction in retinal thickness or the longer period of time before thickening of the retina, the more efficacious the treatment. Retinal function may be determined, for example, by ERG. ERG is a non-invasive electrophysiologic test of retinal function, approved by the FDA for use in humans, which examines the light sensitive cells of the eye (the rods and cones), and their connecting ganglion cells, in particular, their response to a flash stimulation. Retinal thickness may be determined, for example, by SD-OCT. SD-OCT is a three-dimensional imaging technology which uses low-coherence interferometry to determine the echo time delay and magnitude of backscattered light reflected off an object of interest. OCT can be used to scan the layers of a tissue sample (e.g., the retina) with 3 to 15 μm axial resolution, and SD-OCT improves axial resolution and scan speed over previous forms of the technology (Schuman, 2008, Trans. Am. Opthamol. Soc. 106:426-458).

Effects of the methods provided herein may also be measured by a change from baseline in National Eye Institute Visual Functioning Questionnaire, the Rasch-scored version (NEI-VFQ-28-R) (composite score; activity limitation domain score; and socio-emotional functioning domain score). Effects of the methods provided herein may also be measured by a change from baseline in National Eye Institute Visual Functioning Questionnaire 25-item version (NEI-VFQ-25) (composite score and mental health subscale score.

In specific embodiments, the efficacy of a method described herein is reflected by an improvement in vision at about 4 weeks, 12 weeks, 6 months, 12 months, 24 months, 36 months, or at other desired timepoints. In a specific embodiment, the improvement in vision is characterized by an increase in BCVA, for example, an increase by 1 letter, 2 letters, 3 letters, 4 letters, 5 letters, 6 letters, 7 letters, 8 letters, 9 letters, 10 letters, 11 letters, or 12 letters, or more. In a specific embodiment, the improvement in vision is characterized by a 5%, 10%, 15%, 20%, 30%, 40%, 50% or more increase in visual acuity from baseline.

In specific embodiments, the efficacy of a method described herein is reflected by an reduction in central retinal thickness (CRT) at about 4 weeks, 12 weeks, 6 months, 12 months, 24 months, 36 months, or at other desired timepoint, for example, a 5%, 10%, 15%, 20%, 30%, 40%, 50% or more decrease in central retinal thickness from baseline.

In specific embodiments, there is no inflammation in the eye after treatment or little inflammation in the eye after treatment (for example, an increase in the level of inflammation by 10%, 5%, 2%, 1% or less from baseline).

In specific embodiments, the proportion of subjects who experience ocular inflammation following administration of a steroid treatment followed by surabgene lomparvovec treatment and continued steroid treatment as described herein is less than three-quarters, less than half, less than one-quarter, or less than one-tenth of all subjects in a population of subjects. In specific embodiments, the proportion of subjects who experience ocular inflammation following administration of surabgene lomparvovec treatment and a steroid treatment described herein is reduced by about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100% as compared to the proportion in a reference population. In specific embodiments, the proportion of subjects who experience ocular inflammation following administration of surabgene lomparvovec treatment and a steroid treatment described herein is reduced by between about 5% and about 10%, about 10% and about 15%, about 15% and about 20%, about 20% and about 25%, about 25% and about 30%, about 30% and about 35%, about 35% and about 40%, about 40% and about 45%, about 45% and about 50%, about 50% and about 55%, about 55% and about 60%, about 60% and about 65%, about 65% and about 70%, about 70% and about 75%, about 75% and about 80%, about 80% and about 85%, about 85% and about 90%, about 90% and about 95%, or about 95% and about 100% as compared to the proportion in a reference population. In certain embodiments, the reference population consists of individuals administered surabgene lomparvovec for treatment of neovascular age-related macular generation or diabetic retinopathy, but who have received concomitant steroids (i.e., concurrently with the surabgene lomparvovec administration) as opposed to a prophylactic (i.e., beginning at least about three days prior to the administration of the surabgene lomparvovec) steroid treatment described herein, for example, about three days prior, or about four days prior, or about five days prior, or about six days prior, or about seven days prior to the administration of the surabgene lomparvovec. In this context, about three days includes from 2.5 days to less than 3.5 days, about four days includes from 3.5 days to less than 4.5 days, and so on.

Vector shedding may be determined for example by measuring vector DNA in biological fluids such as tears, serum or urine using quantitative polymerase chain reaction. In some embodiments, no vector gene copies are detectable in urine at any time point after administration of the vector. In some embodiments, less than 1000, less than 500, less than 100, less than 50 or less than 10 vector gene copies/5 μL are detectable by quantitative polymerase chain reaction in a biological fluid (e.g., tears, serum or urine) at any point after administration. In specific embodiments, 210 vector gene copies/5 μL or less are detectable in serum. In some embodiments, less than 1000, less than 500, less than 100, less than 50 or less than 10 vector gene copies/5 μL are detectable by quantitative polymerase chain reaction in a biological fluid (e.g., tears, serum or urine) by 1, 2, 3, 4, 5, 6, 7, 8, 9, 10, 11, 12, 13 or 14 weeks after administration. In specific embodiments, no vector gene copies are detectable in a biological fluid (e.g., tears, serum or urine) by Week 14 after administration of the vector. In some embodiments, no vector gene copies are detectable in a biological fluid (e.g., tears, serum or urine) at any time point after administration of the vector.

In some embodiments, patients treated in accordance with a method provided herein are monitored for the development of Center Involved-Diabetic Macular Edema (CI-DME), cataracts, neovascularization, retinal detachment, diabetes complications, vessel regression, area of leakage, and/or area of retinal nonperfusion. Development of CI-DME, cataracts, neovascularization, retinal detachment, diabetes complications, vessel regression, area of leakage, and area of retinal nonperfusion may be assessed by any method known in the art or provided herein. Diabetic complications developed in a subject may require panretinal photocoagulation (PRP), anti-VEGF therapy and/or surgical intervention). Diabetic complications may be sight-threatening. Cataracts developed in a subject may require surgery. In some embodiments, the vital signs (e.g., heart rate, blood pressure) of a patient treated in accordance with the methods provided herein may be monitored.

The safety of a method of treatment described herein may be assessed by assays known in the art. In certain embodiments, the safety of a method of treatment described herein is assessed by serum chemistry measurements of, e.g., levels of glucose, blood urea nitrogen, creatinine, sodium, potassium, chloride, carbon dioxide, calcium, total protein albumin total bilirubin, direct bilirubin, alkaline phosphatase, alanine aminotransferase, aspartate aminotransferase, and/or creatine kinase. In certain embodiments, the safety of a method of treatment described herein is assessed by hematological measurements of, e.g., platelets, hematocrit, hemoglobin, red blood cells, white blood cells, neutrophils, lymphocytes, monocytes, eosinophils, basophils, mean corpuscular volume, mean corpuscular hemoglobin and/or mean corpuscular hemoglobin concentration. In certain embodiments, the safety of a method of treatment described herein is assessed by urinalysis, e.g., a dipstick test for levels of glucose, ketones, protein, and/or blood (if warranted, a microscopic evaluation may be completed). In certain embodiments, the safety of a method of treatment described herein is assessed by measurements of coagulation (e.g., prothrombin time and/or partial thromboplastin time) or by measurements of hemoglobin A1c.

In certain embodiments, the effects of a method provided herein are determined by statistical analysis. Statistical inference may be done at a significance level of 2-sided α=0.2. Statistical endpoints may be summarized with a corresponding 80% confidence interval.

The effects of a method provided herein may be determined by Fisher's Exact test, wherein a treated population is tested against a historical rate of response (e.g., 5%) in an untreated population.

### 6.2.5 Steroid Regimens

In certain embodiments, the steroid treatment comprises topically administering a steroid.

In certain embodiments, topical administration of the steroid ameliorates or prevents ocular inflammation after administration of surabgene lomparvovec. In certain embodiments, topical administration of the steroid ameliorates ocular inflammation. In certain embodiments, topical administration of the steroid prevents ocular inflammation. In certain embodiments, topical administration of the steroid ameliorates or prevents intraocular inflammation. In certain embodiments, topical administration of the steroid ameliorates intraocular inflammation. In certain embodiments, topical administration of the steroid prevents intraocular inflammation. In certain embodiments, topical administration of the steroid ameliorates or prevents episcleritis. In certain embodiments, topical administration of the steroid ameliorates episcleritis. In certain embodiments, topical administration of the steroid prevents episcleritis.

In certain embodiments, the steroid treatment comprises topically administering a corticosteroid. In certain embodiments, the corticosteroid is selected from the group consisting of prednisolone, dexamethasone, and difluprednate, or pharmaceutically acceptable salts thereof. In certain embodiments, the corticosteroid is prednisolone or a pharmaceutically acceptable salt thereof. In certain embodiments, the corticosteroid is dexamethasone or a pharmaceutically acceptable salt thereof. In certain embodiments, the corticosteroid is difluprednate or a pharmaceutically acceptable salt thereof. In certain embodiments, the steroid treatment comprises administering prednisolone or a pharmaceutically acceptable salt thereof. In certain embodiments, the steroid treatment comprises administering dexamethasone or a pharmaceutically acceptable salt thereof. In certain embodiments, the steroid treatment comprises administering difluprednate or a pharmaceutically acceptable salt thereof.

In some embodiments, the steroid treatment is a steroid regimen which begins three days before administration of the surabgene lomparvovec with daily administration of the steroids. In some embodiments, following administration of surabgene lomparvovec, the daily steroid administration continues for 7 to 12 weeks, or for 7 to 14 weeks, or for 7 to 16 weeks, or for 7 to 20 weeks. In some embodiments, during those 7 to 12 weeks or 7 to 14 weeks or 7 to 16 weeks or 7 to 20 weeks, the dosing of the steroid is decreased to taper down the amount of steroid by decreasing the numbers of times per day the steroid is administered and/or by administering the steroid every other day.

In some embodiments, the steroid treatment is a steroid regimen which begins six to seven days before administration of the surabgene lomparvovec with daily administration of the steroids. In some embodiments, following administration of surabgene lomparvovec, the daily steroid administration continues for 7 to 12 weeks, or for 7 to 14 weeks, or 7 to 16 weeks, or for 7 to 20 weeks. In some embodiments, during those 7 to 12 weeks or 7 to 14 weeks or 7 to 16 weeks or 7 to 20 weeks, the dosing of the steroid is decreased to taper down the amount of steroid by decreasing the numbers of times per day the steroid is administered and/or by administering the steroid every other day.

In certain embodiments, difluprednate or a pharmaceutically acceptable salt thereof is provided as DUREZOL® (0.05% difluprednate ophthalmic emulsion). In certain embodiments, prednisolone or a pharmaceutically acceptable salt thereof is provided as PRED FORTE® (1% prednisolone acetate ophthalmic suspension). In certain embodiments, dexamethasone or a pharmaceutically acceptable salt thereof is provided as MAXIDEX® (0.1% dexamethasone ophthalmic solution) dexamethasone sodium phosphate 0.1% ophthalmic solution, or dexamethasone phosphate 0.1% ophthalmic solution.

In certain embodiments, difluprednate (e.g., DUREZOL®) is first administered prior to administration of surabgene lomparvovec. In certain embodiments, difluprednate is first administered one, two, three, four, five, six or seven days prior to administration of surabgene lomparvovec. In certain embodiments, difluprednate is administered using 1 drop 4 times daily in the eye for 3 days prior to administration of surabgene lomparvovec, or for 4, 5, 6 or 7 days prior to administration of surabgene lomparvovec, 1 drop in the eye the day of surabgene lomparvovec administration, then 1 drop 4 times daily for 4 weeks, followed by 1 drop 3 times daily for 1 week, followed by 1 drop 2 times daily for 1 week, and followed by 1 drop 1 time daily for 1 week. In certain embodiments, difluprednate is administered using 1 drop 4 times daily in the eye for 3 days prior to administration of surabgene lomparvovec, or for 4, 5, 6 or 7 days prior to administration of surabgene lomparvovec, then 1 drop 4 times daily for 4 weeks including the day of administration, followed by 1 drop 3 times daily for 2 weeks, followed by 1 drop 2 times daily for 2 weeks, followed by 1 drop 1 time daily for 2 weeks, and followed by 1 drop 1 time daily every other day for 2 weeks. In certain embodiments, difluprednate is administered using 1 drop 6 times daily in the eye for 3, 4, 5, 6 or 7 days prior to administration of surabgene lomparvovec, then 1 drop 6 times daily for 2 weeks including the day of administration, followed 1 drop 4 times daily for 2 weeks, followed by 1 drop 3 times daily for 2 weeks, followed by 1 drop 2 times daily for 2 weeks, followed by 1 drop 1 time daily for 2 week, and followed by 1 drop 1 time every other day for 2 weeks. In certain embodiments, difluprednate is administered using 1 drop 4 times daily in the eye for 3, 4, 5, 6 or 7 days prior to administration of surabgene lomparvovec, then beginning Day 2 (the day immediately after the day of the administration of the dose of surabgene lomparvovec), 1 drop 4 times daily for 4 weeks, followed by 1 drop 3 times daily for 2 weeks, followed by 1 drop 2 times daily for 2 weeks, followed by 1 drop 1 time daily for 2 weeks, and followed by 1 drop 1 time daily every other day for 2 weeks. In some embodiments, the method further includes administering 1 drop of the difluprednate or a pharmaceutically acceptable salt thereof to the eye of said subject during the day of, but after, the administration of the dose of surabgene lomparvovec. In certain embodiments, difluprednate is administered using 1 drop 4 times daily in the eye for 3, 4, 5, 6 or 7 days prior to administration of surabgene lomparvovec, then beginning Day 2 (the day immediately after the day of the administration of the dose of surabgene lomparvovec), 1 drop 4 times daily for 5 weeks, followed by 1 drop 3 times daily for 3 weeks, followed by 1 drop 2 times daily for 3 weeks, followed by 1 drop once daily for 3 weeks, and followed by 1 drop 1 time every other day for 2 weeks. In some embodiments, the method further includes administering 1 drop of the difluprednate or a pharmaceutically acceptable salt thereof to the eye of said subject during the day of, but after, the administration of the dose of surabgene lomparvovec.

In certain embodiments, prednisolone or a pharmaceutically acceptable salt thereof (e.g., PRED FORTE®) is first administered prior to administration of surabgene lomparvovec. In certain embodiments, prednisolone is first administered one, two, three, four, five, six or seven days prior to administration of surabgene lomparvovec. In certain embodiments, prednisolone is administered using 2 drops 4 times daily in the eye for 3 days prior to administration of surabgene lomparvovec, or for 4, 5, 6 or 7 days prior to administration of surabgene lomparvovec, 2 drops in the eye the day of surabgene lomparvovec administration, then 2 drops 4 times daily for 4 weeks, followed by 2 drops 3 times daily for 1 week, followed by 2 drops 2 times daily for 1 week, and followed by 2 drops 1 time daily for 1 week. In certain embodiments, prednisolone is administered using 2 drops 4 times daily in the eye for 3 days prior to administration of surabgene lomparvovec, or for 4, 5, 6 or 7 days prior to administration of surabgene lomparvovec, then 2 drops 4 times daily for 4 weeks including the day of administration, followed by 2 drops 3 times daily for 2 weeks, followed by 2 drops 2 times daily for 2 weeks, followed by 2 drops 1 time daily for 2 weeks, and followed by 2 drops 1 time daily every other day for 2 weeks. In certain embodiments, prednisolone is administered using 2 drops 6 times daily in the eye for 3 days prior to administration of surabgene lomparvovec, or for 4, 5, 6 or 7 days prior to administration of surabgene lomparvovec, then 2 drops 6 times daily for 4 weeks including the day of administration, followed by 2 drops 5 times daily for 1 week, followed by 2 drops 4 times daily for 1 week, followed by 2 drops 3 times daily for 1 week, followed by 2 drops 2 times daily for 1 week, followed by 2 drops 1 time daily for 2 weeks, and followed by 2 drops 1 time every other day for 2 weeks. In some embodiments, the method further includes administering 1 drop of the difluprednate or a pharmaceutically acceptable salt thereof to the eye of said subject during the day of, but after, the administration of the dose of surabgene lomparvovec. In certain embodiments, prednisolone is administered using 2 drops 8 times daily in the eye for 3, 4, 5, 6 or 7 days prior to administration of surabgene lomparvovec, then 2 drops 8 times daily for 2 weeks including the day of administration, followed by 2 drops 6 times daily for 2 weeks, followed by 2 drops 4 times daily for 2 weeks, followed by 2 drops 3 times daily for 2 weeks, followed by 2 drops 2 times daily for 2 weeks, followed by 2 drops 1 time daily for 1 week, and followed by 2 drops 1 time every other day for 1 week. In certain embodiments, prednisolone is administered using 2 drops 4 times daily in the eye for 3, 4, 5, 6 or 7 days prior to administration of surabgene lomparvovec, then beginning the day of the administration of the dose of surabgene lomparvovec, 2 drops 4 times daily for 4 weeks, followed by 2 drops 3 times daily for 2 weeks, followed by 2 drops 2 times daily for 2 weeks, followed by 2 drops 1 time daily for 2 weeks, and followed by 2 drops 1 time daily every other day for 2 weeks. In certain embodiments, prednisolone is administered using 2 drops 6 times daily in the eye for 3, 4, 5, 6 or 7 days prior to administration of surabgene lomparvovec, then beginning Day 2 (the day immediately after the day of the administration of the dose of surabgene lomparvovec), 2 drops 6 times daily for 4 weeks, followed by 2 drops 5 times daily for 1 week, followed by 2 drops 4 times daily for 1 week, followed by 2 drops 3 times daily for 1 week, followed by 2 drops 2 times daily for 1 week, followed by 2 drops once daily for 2 weeks, and followed by 2 drops 1 time every other day for 2 weeks. In some embodiments, the method further includes administering 1 drop of the difluprednate or a pharmaceutically acceptable salt thereof to the eye of said subject during the day of, but after, the administration of the dose of surabgene lomparvovec. In certain embodiments, prednisolone is administered using 2 drops 6 times daily in the eye for 3, 4, 5, 6 or 7 days prior to administration of surabgene lomparvovec, then beginning the day of the administration of the dose of surabgene lomparvovec, 2 drops 6 times daily for 5 weeks, followed by 2 drops 5 times daily for 2 weeks, followed by 2 drops 4 times daily for 2 weeks, followed by 2 drops 3 times daily for 2 weeks, followed by 2 drops 2 times daily for 2 weeks, followed by 2 drops once daily for 1 week, and followed by 2 drops 1 time every other day for 2 weeks. In some embodiments, the method further includes administering 1 drop of the difluprednate or a pharmaceutically acceptable salt thereof to the eye of said subject during the day of, but after, the administration of the dose of surabgene lomparvovec. Surprisingly, the inventors found topical administration of prednisolone or a pharmaceutically acceptable salt thereof as described herein surprisingly shows a comparable or improved amelioration or prevention of ocular inflammation compared to topical administration of difluprednate or a pharmaceutically acceptable salt thereof as described herein. In certain embodiments, administration of prednisolone or a pharmaceutically acceptable salt thereof includes waiting up to 5 minutes after administering a first drop of prednisolone to administer a second drop of prednisolone. In certain embodiments, administration of prednisolone or a pharmaceutically acceptable salt thereof includes waiting 5 minutes to 10 minutes after administering a first drop of prednisolone to administer a second drop of prednisolone. In certain embodiments, administration of prednisolone or a pharmaceutically acceptable salt thereof includes waiting 5 minutes after administering a first drop of prednisolone to administer a second drop of prednisolone.

In certain embodiments, dexamethasone (e.g., MAXIDEX®, dexamethasone sodium phosphate 0.1% ophthalmic solution, or dexamethasone phosphate 0.1% ophthalmic solution) is first administered prior to administration of surabgene lomparvovec. In certain embodiments, dexamethasone is first administered one, two, three, four, five, six or seven days prior to administration of surabgene lomparvovec. In certain embodiments, dexamethasone is administered using 2 drops 4 times daily in the eye for 3 days prior to administration of surabgene lomparvovec, or for 4, 5, 6 or 7 days prior to administration of surabgene lomparvovec, 2 drops in the eye the day of surabgene lomparvovec administration, then 2 drops 4 times daily for 4 weeks, followed by 2 drops 3 times daily for 1 week, followed by 2 drops 2 times daily for 1 week, and followed by 2 drops 1 time daily for 1 week. In certain embodiments, dexamethasone is administered using 2 drops 4 times daily in the eye for 3 days prior to administration of surabgene lomparvovec, or for 4, 5, 6 or 7 days prior to administration of surabgene lomparvovec, then 2 drops 4 times daily for 4 weeks including the day of administration, followed by 2 drops 3 times daily for 2 weeks, followed by 2 drops 2 times daily for 2 weeks, followed by 2 drops 1 time daily for 2 weeks, and followed by 2 drops 1 time daily every other day for 2 weeks.

6.2.6 Treatment of Neovascular Age-Related Macular Degeneration (nAMD)

In some embodiments, the methods described herein are useful for treating neovascular age-related macular degeneration, or nAMD.

Age-related macular degeneration (AMD) is a progressive degenerative macular disease attacking the region of highest visual acuity (VA), the macula. The neovascular or “wet” form of the disease (nAMD) is characterized by choroidal neovascularization (CNV), which is marked by proliferation of blood vessels and cells including those of the retinal pigment epithelium (RPE). (See, Carmeliet P. “Angiogenesis in life, disease and medicine.” Nature. 2005; 438 (7070): 932-6). Ultimately, photoreceptor death and scar formation result in a severe loss of central vision and the inability to read, write, recognize faces, or drive. Many patients with the disease progress to the point that they can no longer maintain gainful employment or carry out daily activities and, consequently, report a diminished quality of life. (See, Mitchell J, Bradley C. “Quality of life in age-related macular degeneration: a review of the literature.” Health Qual Life Outcomes. 2006; 4:97). Preventive therapies for nAMD have demonstrated little effect, and therapeutic strategies have focused primarily on treating the neovascular lesion.

Excessive vascular endothelial growth factor (VEGF) plays a key part in promoting neovascularization and edema in nAMD. Counteracting the effects of VEGF provides significant therapeutic benefit to patients suffering from the disorder. (See, AAO PPP. “Age-related Macular Degeneration Preferred Practice Pattern®.” American Academy of Ophthalmology. 2019.) Currently available anti-VEGF agents approved for the treatment of nAMD, such as ranibizumab (LUCENTIS®, Genentech), aflibercept (EYLEA®, Regeneron), or brolucizumab (BEOVU®, Novartis), have been shown to increase best-corrected visual acuity (BCVA) over time (See, Schmidt-Erfurth U, Chong V, Loewenstein A, et al. “Guidelines for the management of neovascular age-related macular degeneration by the European Society of Retina Specialists (EURETINA).” Br J Ophthalmol. 2014; 98 (9): 1144-67; see also, Dugel P U, Koh A, Ogura Y, et al. “HAWK and HARRIER: Phase 3, Multicenter, Randomized, Double-Masked Trials of Brolucizumab for Neovascular Age-Related Macular Degeneration.” Ophthalmology. 2020; 127 (1): 72-84). However, their beneficial effects may be limited in duration of effectiveness. (See, Comparison of Age-related Macular Degeneration Treatments Trials Research Group, Maguire M G, Martin D F, et al. “Five-Year Outcomes with Anti-Vascular Endothelial Growth Factor Treatment of Neovascular Age-Related Macular Degeneration: The Comparison of Age-Related Macular Degeneration Treatments Trials.” Ophthalmology. 2016; 123 (8): 1751-61; see also, Rofagha S, Bhisitkul R B, Boyer D S, et al. “Seven-year outcomes in ranibizumab-treated patients in ANCHOR, MARINA, and HORIZON: a multicenter cohort study (SEVEN-UP).” Ophthalmology. 2013; 120 (11): 2292-9). As described above, surabgene lomparvovec is an AAV8-based gene therapy capable of transducing human retinal cells to produce a protein almost identical to ranibizumab ((LUCENTIS®).

In some embodiments, the method of treating nAMD comprises administering surabgene lomparvovec treatment and a steroid treatment in a regimen described herein.

In some embodiments, the method of treating nAMD comprises administering surabgene lomparvovec treatment and a steroid treatment, wherein (A) the surabgene lomparvovec treatment comprises administering a therapeutically effective amount of surabgene lomparvovec to the suprachoroidal space of an eye of the subject; and (B) the steroid treatment comprises administering a therapeutically effective amount of a steroid to the eye of the subject about three days prior to administration of the surabgene lomparvovec.

In some embodiments of the method of treating nAMD, the steroid administration the begins three days before administration of the surabgene lomparvovec continues for about 7 to 12 weeks, or for about 7 to 14 weeks, or for about 7 to 20 weeks, after administration of the surabgene lomparvovec in a tapering regimen as described in the Steroid Regimens section above.

In some embodiments of the method of treating nAMD, the steroid administration the begins six to seven days before administration of the surabgene lomparvovec continues for about 7 to 12 weeks, or for about 7 to 14 weeks, or for about 7 to 20 weeks, after administration of the surabgene lomparvovec in a tapering regimen as described in the Steroid Regimens section above.

In certain embodiments of the method of treating nAMD provided herein, the treatment comprises administering surabgene lomparvovec to the suprachoroidal space of an eye of the subject; and the steroid treatment comprises administering difluprednate to the eye of the subject. In certain embodiments, the difluprednate is administered daily to the eye of the subject. In certain embodiments, the steroid treatment comprises administering difluprednate four times daily. In certain embodiments, the difluprednate is administered four times daily for at least one week, at least two weeks, at least three weeks, or at least four weeks. In a specific embodiment, the difluprednate is administered four times daily for about four weeks. In certain embodiments, the steroid treatment comprises administering difluprednate three times daily. In certain embodiments, the difluprednate is administered three times daily for at least one day, at least two days, at least three days, at least four days, at least five days, at least six days, or at least one week. In a specific embodiment, the difluprednate is administered three times daily for about one week. In certain embodiments, the steroid treatment comprises administering difluprednate two times daily. In certain embodiments, the difluprednate is administered two times daily for at least one day, at least two days, at least three days, at least four days, at least five days, at least six days, or at least one week. In a specific embodiment, the difluprednate is administered two times daily for about one week. In certain embodiments, the steroid treatment comprises administering difluprednate one time daily. In certain embodiments, the difluprednate is administered one time daily for at least one day, at least two days, at least three days, at least four days, at least five days, at least six days, or at least one week. In a specific embodiment, the difluprednate is administered one time daily for about one week. In certain embodiments, the difluprednate is administered to the eye of the subject for a period of at least one week, at least two weeks, at least three weeks, at least four weeks, at least five weeks, at least six weeks, or at least seven weeks. In certain embodiments, the difluprednate is administered to the eye of the subject for a period of at least one week, at least two weeks, at least three weeks, at least four weeks, at least five weeks, at least six weeks, at least seven weeks, at least eight weeks, at least nine weeks, at least ten weeks, at least eleven weeks, at least twelve weeks, at least thirteen weeks, at least fourteen weeks, at least fifteen weeks, at least sixteen weeks, at least seventeen weeks, at least eighteen weeks, at least nineteen weeks or at least twenty weeks. In a specific embodiment, the difluprednate is administered to the eye of the subject for a period of about seven weeks. In a specific embodiment, the difluprednate is administered to the eye of the subject for a period of about twelve weeks. In another specific embodiment, the steroid treatment comprises administering difluprednate once on the first day of the steroid treatment, followed by four times daily for about four weeks, followed by three times daily for about one week, followed by two times daily for about one week, followed by one time daily for about one week. In yet another specific embodiment, the steroid treatment consists of administering difluprednate four times daily for three days prior to surabgene lomparvovec treatment, followed by four times daily for about four weeks, followed by three times daily for about one week, followed by two times daily for about one week, followed by one time daily for about one week. In certain embodiments, the difluprednate is administered in the form of an ophthalmic emulsion. In certain embodiments, the ophthalmic emulsion comprises 0.5 mg/mL (0.05%) difluprednate. In certain embodiments, each administration of difluprednate comprises instilling one drop of the ophthalmic emulsion in the eye of the subject. In certain embodiments, each administration of difluprednate consists of instilling one drop of the ophthalmic emulsion in the eye of the subject.

In certain embodiments of the method of treating nAMD provided herein, the treatment comprises administering surabgene lomparvovec to the suprachoroidal space of an eye of the subject; and the steroid treatment comprises administering prednisolone to the eye of the subject. In certain embodiments, the prednisolone is administered daily to the eye of the subject. In certain embodiments, the steroid treatment comprises administering prednisolone four times daily. In certain embodiments, the prednisolone is administered four times daily for at least one week, at least two weeks, at least three weeks, or at least four weeks. In a specific embodiment, the prednisolone is administered four times daily for about four weeks. In certain embodiments, the steroid treatment comprises administering prednisolone three times daily. In certain embodiments, the prednisolone is administered three times daily for at least one day, at least two days, at least three days, at least four days, at least five days, at least six days, or at least one week. In a specific embodiment, the prednisolone is administered three times daily for about one week. In certain embodiments, the steroid treatment comprises administering prednisolone two times daily. In certain embodiments, the prednisolone is administered two times daily for at least one day, at least two days, at least three days, at least four days, at least five days, at least six days, or at least one week. In a specific embodiment, the prednisolone is administered two times daily for about one week. In certain embodiments, the steroid treatment comprises administering prednisolone one time daily. In certain embodiments, the prednisolone is administered one time daily for at least one day, at least two days, at least three days, at least four days, at least five days, at least six days, or at least one week. In a specific embodiment, the prednisolone is administered one time daily for about one week. In certain embodiments, the prednisolone is administered to the eye of the subject for a period of at least one week, at least two weeks, at least three weeks, at least four weeks, at least five weeks, at least six weeks, or at least seven weeks. In certain embodiments, the prednisolone is administered to the eye of the subject for a period of at least one week, at least two weeks, at least three weeks, at least four weeks, at least five weeks, at least six weeks, at least seven weeks, at least eight weeks, at least nine weeks, at least ten weeks, at least eleven weeks, at least twelve weeks, at least thirteen weeks, at least fourteen weeks, at least fifteen weeks, at least sixteen weeks, at least seventeen weeks, at least eighteen weeks, at least nineteen weeks or at least twenty weeks. In a specific embodiment, the prednisolone is administered to the eye of the subject for a period of about seven weeks. In a specific embodiment, the prednisolone is administered to the eye of the subject for a period of about twelve weeks. In another specific embodiment, the steroid treatment comprises administering prednisolone once on the first day of the steroid treatment, followed by four times daily for about four weeks, followed by three times daily for about one week, followed by two times daily for about one week, followed by one time daily for about one week. In yet another specific embodiment, the steroid treatment consists of administering prednisolone four times daily for three days prior to surabgene lomparvovec treatment, followed by four times daily for about four weeks, followed by three times daily for about one week, followed by two times daily for about one week, followed by one time daily for about one week. In certain embodiments, the prednisolone is administered in the form of an ophthalmic suspension. In certain embodiments, the ophthalmic suspension comprises 1% prednisolone acetate. In certain embodiments, each administration of prednisolone comprises instilling two drops of the ophthalmic suspension in the eye of the subject. In certain embodiments, each administration of prednisolone consists of instilling two drops of the ophthalmic suspension in the eye of the subject.

In certain embodiments of the method of treating nAMD provided herein, the treatment comprises administering surabgene lomparvovec to the suprachoroidal space of an eye of the subject; and the steroid treatment comprises administering dexamethasone to the eye of the subject. In certain embodiments, the dexamethasone is administered daily to the eye of the subject. In certain embodiments, the steroid treatment comprises administering dexamethasone four times daily. In certain embodiments, the dexamethasone is administered four times daily for at least one week, at least two weeks, at least three weeks, or at least four weeks. In a specific embodiment, the dexamethasone is administered four times daily for about four weeks. In certain embodiments, the steroid treatment comprises administering dexamethasone three times daily. In certain embodiments, the dexamethasone is administered three times daily for at least one day, at least two days, at least three days, at least four days, at least five days, at least six days, or at least one week. In a specific embodiment, the dexamethasone is administered three times daily for about one week. In certain embodiments, the steroid treatment comprises administering dexamethasone two times daily. In certain embodiments, the dexamethasone is administered two times daily for at least one day, at least two days, at least three days, at least four days, at least five days, at least six days, or at least one week. In a specific embodiment, the dexamethasone is administered two times daily for about one week. In certain embodiments, the steroid treatment comprises administering dexamethasone one time daily. In certain embodiments, the dexamethasone is administered one time daily for at least one day, at least two days, at least three days, at least four days, at least five days, at least six days, or at least one week. In a specific embodiment, the dexamethasone is administered one time daily for about one week. In certain embodiments, the dexamethasone is administered to the eye of the subject for a period of at least one week, at least two weeks, at least three weeks, at least four weeks, at least five weeks, at least six weeks, or at least seven weeks. In certain embodiments, the dexamethasone is administered to the eye of the subject for a period of at least one week, at least two weeks, at least three weeks, at least four weeks, at least five weeks, at least six weeks, at least seven weeks, at least eight weeks, at least nine weeks, at least ten weeks, at least eleven weeks, at least twelve weeks, at least thirteen weeks, at least fourteen weeks, at least fifteen weeks, at least sixteen weeks, at least seventeen weeks, at least eighteen weeks, at least nineteen weeks or at least twenty weeks. In a specific embodiment, the dexamethasone is administered to the eye of the subject for a period of about seven weeks. In a specific embodiment, the dexamethasone is administered to the eye of the subject for a period of about twelve weeks. In another specific embodiment, the steroid treatment comprises administering dexamethasone once on the first day of the steroid treatment, followed by four times daily for about four weeks, followed by three times daily for about one week, followed by two times daily for about one week, followed by one time daily for about one week. In yet another specific embodiment, the steroid treatment consists of administering dexamethasone four times daily for three days prior to surabgene lomparvovec treatment, followed by four times daily for about four weeks, followed by three times daily for about one week, followed by two times daily for about one week, followed by one time daily for about one week. In certain embodiments, the dexamethasone is administered in the form of an ophthalmic solution. In certain embodiments, the ophthalmic solution comprises 0.1% dexamethasone sodium phosphate. In certain embodiments, each administration of dexamethasone comprises instilling two drops of the ophthalmic solution in the eye of the subject. In certain embodiments, each administration of dexamethasone consists of instilling two drops of the ophthalmic solution in the eye of the subject.

In some embodiments of the method of treating nAMD, the steroid treatment enables administration of surabgene lomparvovec at doses equal to or greater than about 1×10¹² GC/eye. In some embodiments of the method of treating nAMD, the steroid treatment enables administration of surabgene lomparvovec at doses equal to or greater than about 5×10¹¹ GC/eye.

In some embodiments of the method of treating nAMD, surabgene lomparvovec is administered at a dose of between about 5×10¹¹ genome copies per eye and about 2×10¹² genome copies per eye. In certain embodiments, surabgene lomparvovec is administered at a dose of about 5×10¹¹ genome copies per eye. In certain embodiments, surabgene lomparvovec is administered at a dose of about 1×10¹² genome copies per eye. In certain embodiments, surabgene lomparvovec is administered at a dose of about 1.5×10¹² genome copies per eye. In certain embodiments, surabgene lomparvovec is administered at a dose of about 2×10¹² genome copies per eye.

In certain embodiments of the method of treating nAMD, surabgene lomparvovec is administered by a single suprachoroidal injection. In certain embodiments, administration of the steroid as described above ameliorates or prevents ocular inflammation. In certain embodiments, administration of the steroid as described above ameliorates or prevents intraocular inflammation. In certain embodiments, administration of the steroid as described above ameliorates or prevents episcleritis. In certain embodiments, administration of the steroid as described above ameliorates or prevents ocular inflammation associated with the dose of surabgene lomparvovec, the number of suprachoroidal injections, and/or the location of suprachoroidal injections. In certain embodiments, administration of the steroid as described above ameliorates or prevents intraocular inflammation associated with the dose of surabgene lomparvovec, the number of suprachoroidal injections, and/or the location of suprachoroidal injections. In certain embodiments, administration of the steroid as described above ameliorates or prevents episcleritis associated with the dose of surabgene lomparvovec, the number of suprachoroidal injections, and/or the location of suprachoroidal injections. In certain embodiments, the steroid treatment comprises administering a therapeutically effective amount of a corticosteroid. In certain embodiments, the steroid treatment comprises topically administering a therapeutically effective amount of a steroid, for example, a corticosteroid. In certain embodiments, the corticosteroid is selected from the group consisting of prednisolone, dexamethasone, and difluprednate. In certain embodiments, the corticosteroid is prednisolone. In certain embodiments, the corticosteroid is dexamethasone. In certain embodiments, the corticosteroid is difluprednate.

In certain embodiments of the method of treating nAMD, surabgene lomparvovec is administered at a dose of at least about 5×10¹¹ genome copies per eye. In certain embodiments, surabgene lomparvovec is administered at a dose of between about 5×10¹¹ genome copies per eye and about 2×10¹² genome copies per eye. In certain embodiments, surabgene lomparvovec is administered at a dose of at least about 5×10¹¹ genome copies per eye. In certain embodiments, surabgene lomparvovec is administered at a dose of about 1×10¹² genome copies per eye. In certain embodiments, surabgene lomparvovec is administered at a dose of about 1.5×10¹² genome copies per eye. In certain embodiments, surabgene lomparvovec is administered at a dose of about 2×10¹² genome copies per eye. In certain embodiments, surabgene lomparvovec is administered by a single suprachoroidal injection. In certain embodiments, topical administration of the steroid as described above ameliorates or prevents ocular inflammation. In certain embodiments, topical administration of the steroid as described above ameliorates or prevents intraocular inflammation. In certain embodiments, topical administration of the steroid as described above ameliorates or prevents episcleritis. In certain embodiments, topical administration of the steroid as described above prevents ocular inflammation. In certain embodiments, topical administration of the steroid as described above prevents intraocular inflammation. In certain embodiments, topical administration of the steroid prevents episcleritis. In certain embodiments, topical administration of the steroid as described above ameliorates or prevents ocular inflammation associated with the dose of surabgene lomparvovec, the number of suprachoroidal injections, and/or the location of suprachoroidal injections. In certain embodiments, topical administration of the steroid as described above ameliorates or prevents intraocular inflammation associated with the dose of surabgene lomparvovec, the number of suprachoroidal injections, and/or the location of suprachoroidal injections. In certain embodiments, topical administration of the steroid as described above ameliorates or prevents episcleritis associated with the dose of surabgene lomparvovec, the number of suprachoroidal injections, and/or the location of suprachoroidal injections.

In certain embodiments of the method of treating nAMD, the steroid treatment comprises topically administering a therapeutically effective amount of a corticosteroid. In certain embodiments, the topical corticosteroid is selected from the group consisting of prednisolone, dexamethasone, and difluprednate. In certain embodiments, the topical corticosteroid is prednisolone. In certain embodiments, the topical corticosteroid is dexamethasone. In certain embodiments, the topical corticosteroid is difluprednate.

In some embodiments of the method of treating nAMD, the method results in an efficacy result, a safety result, or both.

In some embodiments of the method of treating nAMD, the efficacy result is selected from the group consisting of (1) the subject having a reduction in choroidal neovascularization (CNV) lesion size and/or leakage area relative to baseline, (2) the subject having a gain of visual acuity relative to baseline, (3) the subject not having a significant loss of visual acuity relative to baseline, (4) the subject having a reduction in central retinal thickness (CRT) relative to a baseline, (5) the subject showing a reduction in a supplemental anti-VEGF injection annualized rate after the administration of the surabgene lomparvovec, and (6) the subject receiving no more than 2, no more than 1, or no supplemental anti-VEGF injections after the administration of the surabgene lomparvovec. In some embodiments, the baseline is measured and/or determined at the time (i.e., the same day) the surabgene lomparvovec is administered. In some embodiments, the baseline is measured and/or determined 1, 2, 3, 4, 5, 6, or 7 days before the surabgene lomparvovec is administered. In some embodiments, the baseline is measured and/or determined 1, 2, 3, or 4 weeks before the surabgene lomparvovec is administered.

In some embodiments of the method of treating nAMD, the efficacy results are seen within or at 26, 40, or 52 weeks after the administration of the surabgene lomparvovec. In some embodiments, the efficacy result is seen at all or some of those timepoints.

In some embodiments of the method of treating nAMD, the efficacy result is the subject having a reduction in choroidal neovascularization (CNV) lesion size and/or leakage area relative to baseline. In some embodiments of the method of treating nAMD, the reduction in choroidal neovascularization (CNV) lesion size and/or leakage area relative to a baseline CNV lesion size and/or leakage area is within or at 26, 40, or 52 weeks after the administration of the surabgene lomparvovec.

In some embodiments of the method of treating nAMD, the efficacy result is the subject having a gain of visual acuity relative to baseline. In some embodiments of the method of treating nAMD, the gain of visual acuity is a gain of ≥15 letters of BCVA based on the ETDRS scale relative to a baseline number of letters within or at 52, 40, or 26 weeks after the administration of the surabgene lomparvovec. In some embodiments of the method of treating nAMD, the gain of visual acuity is a gain of ≥10 letters of BCVA based on the ETDRS scale relative to a baseline number of letters within or at 52, 40, or 26 weeks after the administration of the surabgene lomparvovec. In some embodiments of the method of treating nAMD, the gain of visual acuity is a gain of ≥5 letters of BCVA based on the ETDRS scale relative to a baseline number of letters within or at 52, 40, or 26 weeks after the administration of the surabgene lomparvovec. In some embodiments of the method of treating nAMD, the gain of visual acuity is a gain of ≥0 letters of BCVA based on the ETDRS scale relative to a baseline number of letters within or at 52, 40, or 26 weeks after the administration of the surabgene lomparvovec.

In some embodiments of the method of treating nAMD, the efficacy result is the subject not having a significant loss of visual acuity relative to baseline. In some embodiments of the method of treating nAMD, the subject does not have a loss of ≥15 letters of BCVA based on the ETDRS scale relative to a baseline number of letters within or at 52, 40, or 26 weeks after the administration of the surabgene lomparvovec. In some embodiments of the method of treating nAMD, the subject does not have a loss of ≥10 letters of BCVA based on the ETDRS scale relative to a baseline number of letters within or at 52, 40, or 26 weeks after the administration of the surabgene lomparvovec. In some embodiments of the method of treating nAMD, the subject does not have a loss of ≥5 letters of BCVA based on the ETDRS scale relative to a baseline number of letters within or at 52, 40, or 26 weeks after the administration of the surabgene lomparvovec. In some embodiments of the method of treating nAMD, the subject does not have a loss of ≥0 letters of BCVA based on the ETDRS scale relative to a baseline number of letters within or at 52, 40, or 26 weeks after the administration of the surabgene lomparvovec.

In some embodiments of the method of treating nAMD, the efficacy result is the subject having a reduction in central retinal thickness (CRT) relative to a baseline. In some embodiments of the method of treating nAMD, the subject has a reduction in central retinal thickness (CRT) relative to a baseline CRT within or at 52, 40, or 26 weeks after the administration of the surabgene lomparvovec. n some embodiments of the method of treating nAMD, the reduction in CRT is no fluid relative to baseline as measured by SD-OCT. In some embodiments of the method of treating nAMD, the reduction in CRT is a fluid level that is within 30 μm of baseline.

Supplemental injections of an anti-VEGF may be necessary in some subjects to treat the nAMD even after the administration of surabgene lomparvovec. However, it is believed that the surabgene lomparvovec administered according the methods of treating nAMD described herein will transduce cells in the subjects retina to produce a protein almost identical to the anti-VEGF agent ranibizumab, thereby reducing the number of anti-VEGF injections the subject would need to receive to treat the nAMD in the absence of the surabgene lomparvovec administration or even eliminate any need for such anti-VEGF injections.

The anti-VEGF used for supplemental injections can be any of anti-VEGF that is used for treating nAMD including, for example, the approved anti-VEGF agents ranibizumab (LUCENTIS®, Genentech), aflibercept (EYLEA®, Regeneron), or brolucizumab (BEOVU®, Novartis) and their biosimilars. Additional anti-VEGF agents may be available in depending on the country.

In some embodiments of the method of treating nAMD where supplemental injections are monitored, the subject may have already been receiving anti-VEGF in injections as treatment for their nAMD. In other embodiments of the method of treating nAMD where supplemental injections are monitored, they may be treatment-naïve with regards to anti-VEGF injections (e.g., the patient may be treated with surabgene lomparvovec as a first treatment for nAMD soon after diagnosis and had never received any anti-VEGF injections prior to administration of the surabgene lomparvovec). In such situations, a such patients may optionally be screened for responsiveness to ranibizumab (or other anti-VEGF agents) prior to surabgene lomparvovec.

In some embodiments of the method of treating nAMD, the subject may specifically receive an initial pre-treatment screening anti-VEGF injection, and in particular, and injection of ranibizumab to test the subject for responsiveness to the anti-VEGF treatment prior to having the surabgene lomparvovec administered. This specifically administered pre-treatment screening anti-VEGF injection is counted as part of the injections for determining the annualized rate of anti-VEGF injections for the 52 weeks prior to the administration of the surabgene lomparvovec.

In some embodiments of the method of treating nAMD, a subject may receive one or more anti-VEGF injections (e.g., ranibizumab) specifically as part of the treatment after administration of the surabgene lomparvovec (e.g., as a means to provide treatment of the nAMD before sufficient numbers of retinal cells have been transduced by the surabgene lomparvovec to produce the transgene protein product. In such situations, the one or more anti-VEGF injections specifically administered as part of the treatment would not count as supplemental anti-VEGF for the purposes of determining the efficacy results involving annualized rates and numbers of supplemental injections.

The percent reduction in supplemental anti-VEGF injection annualized rate for example through Week 40 and Week 52 compared with the prior year, is calculated as: (the annualized anti-VEGF injection rate in the prior year for the study eye−the annualized supplemental anti-VEGF injection rate through Week 40 and Week 52 for the study eye)/(the annualized anti-VEGF injection rate in the prior year for the study eye)×100%.

Thus, in some embodiments of the method of treating nAMD, the efficacy result is the subject showing a reduction in a supplemental anti-VEGF injection annualized rate after the administration of the surabgene lomparvovec. In some embodiments of the method of treating nAMD, the subject shows a reduction of at least 50% or 75% in a supplemental anti-VEGF injection annualized rate through week 40 or 52 after the surabgene lomparvovec administration when compared with the number of anti-VEGF injections during the 52 weeks preceding the surabgene lomparvovec administration, and wherein the subject received at least one anti-VEGF injection during the 52 weeks preceding the surabgene lomparvovec administration. In some embodiments of the method of treating nAMD, the subject shows a reduction of at least 50% or 75% in a supplemental anti-VEGF injection annualized rate through week 40 or 52 after the surabgene lomparvovec administration when compared with the number of anti-VEGF injections during the 52 weeks preceding a pre-treatment screening anti-VEGF injection administration and wherein the subject received at least one anti-VEGF injection during the 52 weeks preceding a pre-treatment screening anti-VEGF administration.

In some embodiments of the method of treating nAMD, the efficacy result is the subject receiving no more than 2, no more than 1, or no supplemental anti-VEGF injections after the administration of the surabgene lomparvovec. In some embodiments of the method of treating nAMD, the subject receives no more than 2, no more than 1, or no supplemental anti-VEGF injections through week 26, 40, or 52 after the surabgene lomparvovec administration.

In some embodiments of the method of treating nAMD, the safety result is mitigation of ocular inflammation of episcleritis, anterior chamber cell, vitritis, iridocyclitis, iritis, eye inflammation, anterior chamber flare, vitreal cells, keratic precipitates, and/or uveitis.

In some embodiments of the method of treating nAMD, the safety result may be seen at the same time points as the efficacy results (e.g., through and/or week 26, 40, or 52 after the surabgene lomparvovec administration). However, in some embodiments of the method of treating nAMD, the safety result may be seen much sooner than the efficacy results.

Thus, in some embodiments of the method of treating nAMD, the safety result can be observed through and/or at 1, 2, 3, 4, 5, 6, or 7 days after administration of the surabgene lomparvovec. In some embodiments of the method to treat nAMD, the safety result can be observed through and/or at 1, 2, 3, or 4 weeks after the administration of the surabgene lomparvovec.

In some embodiments of the method of treating nAMD, the safety effect can be a reduced amount of inflammation (e.g., less episcleritis, anterior chamber cell, vitritis, iridocyclitis, iritis, eye inflammation, anterior chamber flare, vitreal cells, keratic precipitates, and/or uveitis) in a proportion of subjects when compared to a reference population that received concomitant steroids as described in the Sampling and Monitoring Efficiency section above. Accordingly, a subject can have a reduction in the probability of experiencing inflammation (e.g., lower chance of episcleritis, anterior chamber cell, vitritis, iridocyclitis, iritis, eye inflammation, anterior chamber flare, vitreal cells, keratic precipitates, and/or uveitis) that is equivalent and/or proportional to the difference seen between the proportion of subjects treated by the method of treating nAMD described herein and the proportion of subjects in the reference population.

In some embodiments of the method of treating nAMD, the safety effect can be a complete lack of inflammation (e.g., no episcleritis, anterior chamber cell, vitritis, iridocyclitis, iritis, eye inflammation, anterior chamber flare, vitreal cells, keratic precipitates, and/or uveitis) when compared to subjects in the reference population as described in the Sampling and Monitoring Efficiency section above.

As described above, in some embodiments of the method of treating nAMD, the subjects can be screened for responsiveness to ranibizumab prior to the administration of the surabgene lomparvovec by being administered one or more screening injections of ranibizumab prior to the administration of the surabgene lomparvovec and observing the effect of the screening injection on the subject. As also described above, in some embodiments of the method of treating nAMD, a subject can also optionally receive one or more anti-VEGF injections specifically as part of the treatment after administration of the surabgene lomparvovec.

In some embodiments of the method of treating nAMD, the screening ranibizumab injection can be administered about 6 weeks prior to administration of the surabgene lomparvovec, and the biological effect can be observed at some time after the screening ranibizumab injection. In some embodiments of the method of treating nAMD, the observation of the biological effect can be about 1 week after the screening ranibizumab injection is administered. In other embodiments, the screening ranibizumab can be administered 0 weeks prior to the administration of the surabgene lomparvovec (e.g., in cases where it is decided to administer the surabgene lomparvovec to the subject the same day or week they are screened for responsiveness to the screening ranibizumab injection). In other embodiments, the ranibizumab can be administered more than 6 weeks (e.g., 7, 8, 9, or more weeks) or less than 6 weeks (e.g., 5, 4, 3, 2, or 1 week) before the surabgene lomparvovec, although the subject may require conventional anti-VEGF treatment for the nAMD (e.g., via injections of ranibizumab or other antiVEGF agent per the label) if the screening is done more than 6 weeks prior to the eventual surabgene lomparvovec administration due to the need for some form of treatment until the surabgene lomparvovec can be administered and take effect.

The biological effect to be observed as evidence of the subject's responsiveness can be any biological effect known by a person skilled in the art to be associated with anti-VEGF agents like ranibizumab when used to treat nAMD, such as reduced fluid levels in the retina as observed by SD-OCT. Additional biological effects associated with anti-VEGF agents like ranibizumab when used to treat nAMD would be identifiable by a person skilled in the art.

Accordingly, in some embodiments of the method of treating nAMD, the biological effect can be the subject showing no more than 50 μm of remaining fluid height in the area within 3 mm of the parafovea as determined by SD-OCT when compared to the fluid level when the screening ranibizumab injection was administered if the subject at least 50 μm of fluid when the screening ranibizumab injection was administered, or the subject showing any amount of fluid reduction when compared to the fluid level when the screening ranibizumab injection was administered if the subject at less than 50 μm of fluid when the screening ranibizumab injection was administered.

In some embodiments of the method of treating nAMD, the subject may not require any screening injection of ranibizumab because the patient may have previously shown the necessary responsiveness to ranibizumab (e.g., the patient has already been receiving a successful treatment regimen of ranibizumab prior to consideration for treatment with surabgene lomparvovec).

Once the subject's responsiveness to the ranibizumab protein is confirmed, the surabgene lomparvovec can be administered (e.g., about 0, 1, 2, 3, 4, 5, 6, 7, 8, or 9 weeks or more after the administration of the screening ranibizumab injection). However, in some embodiments, a second ranibizumab injection can be administered in between the screening ranibizumab injection and the administration of the surabgene lomparvovec.

In some embodiments of the method of treating nAMD, the second injection of ranibizumab after the screening injection can be administered about 4 weeks after the screening ranibizumab injection. In some embodiments of the method of treating nAMD, the surabgene can be administered about 2 weeks after the second ranibizumab injection (i.e., 6 weeks after the screening ranibizumab injection, or about 5 weeks after the responsiveness was confirmed if it was confirmed about 1 week after the screening ranibizumab injection). This additional injection of ranibizumab after the screening injection is useful because it may take some time for the surabgene lomparvovec to transduce retinal cells to be able to produce therapeutically effective amounts of the transgene antiVEGF product, and in situations where the surabgene lomparvovec won't be administered immediately when the subject's responsiveness is confirmed, the patient will often still require treatment of the nAMD until the surabgene is administered. This additional dose of ranibizumab can be administered a period of time after the injection of the screening ranibizumab injection (e.g., 4 weeks) as per the label of ranibizumab (e.g., the current label for ranibizumab indicates that 3 to 4 monthly—i.e., every 4 weeks-injections should initially be administered to treat nAMD; the screening and second ranibizumab injections would thus be the first two of the 3 or 4 initial injections per the ranibizumab label). The number of weeks between the second ranibizumab injection and the administration of surabgene lomparvovec would be determined by the number of weeks between the screening ranibizumab injection and the administration of surabgene lomparvovec (e.g., if the 4 weeks between initial ranibizumab injections from the ranibizumab label is being followed and the surabgene lomparvovec is administered 5 weeks after the screening ranibizumab injection instead of 6 weeks, then the second ranibizumab injection will be administered 1 week before the surabgene lomparvovec-still 4 weeks after the screening ranibizumab injection-instead of 2 weeks before the surabgene lomparvovec injection). In cases where the surabgene lomparvovec is administered less than 4 weeks after the screening injection, then the second ranibizumab injection would become a post-treatment ranibizumab injection as described below, and in cases where the surabgene lomparvovec is administered more than 8 weeks before the surabgene lomparvovec, then a third (or more) ranibizumab injection can also be administered before administration of the surabgene lomparvovec. In addition, in situations where the ranibizumab is being administered at times indicated on the label that are different that every 4 weeks (e.g., in accordance with the current ranibizumab label, every 3 months after 4 initial monthly injections or less than monthly subject to assessment after 3 monthly initial injections when the patient has already been on ranibizumab therapy prior to screening), then the period of time between any post-screening ranibizumab injections would be determined by that period of time indicated on the label.

In some embodiments of the method of treating nAMD, the subject can receive one or more post-treatment anti-VEGF injections specifically as part of the treatment after administration of the surabgene lomparvovec (e.g., about 2 weeks after the administration of the surabgene lomparvovec). In some embodiments of the method of treating nAMD, the post-treatment anti-VEGF injection is a ranibizumab injection. In some embodiments of the method of treating nAMD, the post-treatment ranibizumab injection is administered about 2 weeks after the surabgene lomparvovec is administered. However, as indicated above, the additional ranibizumab injections (including the second ranibizumab injection after the screening injection) can serve as a way to treat the nAMD while the surabgene lomparvovec is transducing the retinal cells to produce sufficient levels of anti-VEGF transgene product, and the pos-treatment ranibizumab injections can serve this purpose as well. As with the second ranibizumab injection after the screening ranibizumab injection, the post-treatment ranibizumab injection can be administered a period of time after the injection of the second ranibizumab injection that was administered after the screening ranibizumab injection. For example, if the aforementioned 3 or 4 initial monthly (i.e., every 4 weeks) ranibizumab injections (pre the ranibizumab label) are being administered to the subject before and/or after the surabgene lomparvovec is administered, then the first post-treatment ranibizumab injection would be administered 4 weeks after administration of the second ranibizumab injection administered after the screening ranibizumab injection, and that post-treatment injection would be the third in the series (with the option of administering a second post-treatment ranibizumab if the initial series of 4 monthly ranibizumab injections per the ranibizumab label is desired). The period of time between surabgene lomparvovec administration the first (and subsequent) post-treatment ranibizumab injection(s) would be determined by the period of time between the screening ranibizumab injection and the surabgene lomparvovec injection. For example, if the screening injection is administered 6 weeks prior to the surabgene lomparvovec administration with the second ranibizumab after the screening injection administered 4 weeks after the screening injection (or 2 weeks before the surabgene lomparvovec), then if the 4 weeks between ranibizumab injections as per the ranibizumab label is being followed, the first post-treatment ranibizumab injection would be applied 2 weeks after the surabgene lomparvovec administration as this would be 4 weeks after the second ranibizumab injection that was administered after the screening ranibizumab injection (and any second post-treatment ranibizumab injection would be 6 weeks after the surabgene lomparvovec administration, or 4 weeks after the first post-treatment ranibizumab injection). Other periods of time besides 4 weeks between post-treatment ranibizumab injections can be used if indicated by the label of ranibizumab (or other anti-VEGF agent being used). In addition, more than one post-treatment ranibizumab can be administered to the subject. For example, if the subject is administered the surabgene lomparvovec the day of the screening for ranibizumab responsiveness (i.e., the week after the screening ranibizumab injection is administered), then the subject will likely ne up to 3 or 4 or more post-treatment ranibizumab injections (or other anti-VEGF agent) administered with a timing indicated by the label of the ranibizumab (or other anti-VEGF agent).

In some embodiments of the method of treating nAMD, the subject is phakic, at least in the eye to which the surabgene lomparvovec is administered. In other embodiments of the method of treating nAMD, the subject is pseudophakic, at least in the eye to which the surabgene lomparvovec is administered.

6.2.7 Treatment of Diabetic Retinopathy (DR) with or without Center-Involved Diabetic Macular Edema (CI-DME)

In some embodiments, the methods described herein are useful for treating diabetic retinopathy, or DR, in subjects who may or may not also have center-involved diabetic macular edema, or CI-DME.

Diabetic retinopathy ranges from mild nonproliferative disease to severe proliferative disease. The most common early clinically visible manifestations of nonproliferative diabetic retinopathy (NPDR) include microaneurysm formation and intraretinal hemorrhages. Microvascular damage leads to retinal capillary nonperfusion, cotton wool spots, increased numbers of hemorrhages, venous abnormalities, and intraretinal microvascular abnormalities. At any stage in the course of the disease, increased vasopermeability can result in retinal thickening (edema) and/or exudates that may lead to a loss in central visual acuity (VA). The proliferative diabetic retinopathy (PDR) stage results from closure of arterioles and venules with secondary proliferation of new vessels on the retina, optic disc, or anterior segment. Common complications of DR that risk a patient's vision and require either urgent medical or surgical intervention include CI-DME, tractional retinal detachments, epiretinal membranes, and vitreous hemorrhage. The risk of these complications usually increases as the severity of DR increases, although diabetic macular edema (DME) can be present at any stage of DR.2 The link between diabetic ischemia and subsequent proliferation of angiogenic factors including vascular endothelial growth factor (VEGF) has been established.

Although not used widely in clinical practice for DR without DME, intravitreal anti-VEGF therapy (e.g., ranibizumab (LUCENTIS®, Genentech), aflibercept (EYLEA®, Regeneron)) has shown to be effective in patients for reducing the severity of DR. (See, Writing Committee for the Diabetic Retinopathy Clinical Research N, Gross J G, Glassman A R, et al. Panretinal Photocoagulation vs Intravitreous Ranibizumab for Proliferative Diabetic Retinopathy: A Randomized Clinical Trial. JAMA. 2015; 314 (20): 2137-46; see also, Ip M S, Domalpally A, Hopkins J J, et al. Long-term effects of ranibizumab on diabetic retinopathy severity and progression. Arch Ophthalmol. 2012; 130 (9): 1145-52). As described above, surabgene lomparvovec is an AAV8-based gene therapy capable of transducing human retinal cells to produce a protein almost identical to ranibizumab ((LUCENTIS®).

In some embodiments, the method of treating DR with or without CI-DME comprises administering surabgene lomparvovec treatment and a steroid treatment in a regimen described herein.

In some embodiments of treating DR with or without CI-DME, method comprises administering surabgene lomparvovec treatment and a steroid treatment; wherein (A) the surabgene lomparvovec treatment comprises administering a therapeutically effective amount of surabgene lomparvovec to the suprachoroidal space of an eye of the subject; and (B) the steroid treatment comprises administering a therapeutically effective amount of a steroid to the eye of the subject about three days prior to administration of the surabgene lomparvovec.

In some embodiments of the method of treating DR with or without CI-DME, the steroid administration the begins three days before administration of the surabgene lomparvovec continues for about 7 to 12 weeks after administration of the surabgene lomparvovec in a tapering regimen as described in the Steroid Regimens section above.

In certain embodiments of the method of treating DR with or without CI-DME provided herein, the treatment comprises administering surabgene lomparvovec to the suprachoroidal space of an eye of the subject; and the steroid treatment comprises administering difluprednate to the eye of the subject. In certain embodiments, the difluprednate is administered daily to the eye of the subject. In certain embodiments, the steroid treatment comprises administering difluprednate four times daily. In certain embodiments, the difluprednate is administered four times daily for at least one week, at least two weeks, at least three weeks, or at least four weeks. In a specific embodiment, the difluprednate is administered four times daily for about four weeks. In certain embodiments, the steroid treatment comprises administering difluprednate three times daily. In certain embodiments, the difluprednate is administered three times daily for at least one day, at least two days, at least three days, at least four days, at least five days, at least six days, or at least one week. In a specific embodiment, the difluprednate is administered three times daily for about one week. In certain embodiments, the steroid treatment comprises administering difluprednate two times daily. In certain embodiments, the difluprednate is administered two times daily for at least one day, at least two days, at least three days, at least four days, at least five days, at least six days, or at least one week. In a specific embodiment, the difluprednate is administered two times daily for about one week. In certain embodiments, the steroid treatment comprises administering difluprednate one time daily. In certain embodiments, the difluprednate is administered one time daily for at least one day, at least two days, at least three days, at least four days, at least five days, at least six days, or at least one week. In a specific embodiment, the difluprednate is administered one time daily for about one week. In certain embodiments, the difluprednate is administered to the eye of the subject for a period of at least one week, at least two weeks, at least three weeks, at least four weeks, at least five weeks, at least six weeks, or at least seven weeks. In certain embodiments, the difluprednate is administered to the eye of the subject for a period of at least one week, at least two weeks, at least three weeks, at least four weeks, at least five weeks, at least six weeks, at least seven weeks, at least eight weeks, at least nine weeks, at least ten weeks, at least eleven weeks, at least twelve weeks, at least thirteen weeks, at least fourteen weeks, at least fifteen weeks, at least sixteen weeks, at least seventeen weeks, at least eighteen weeks, at least nineteen weeks or at least twenty weeks. In a specific embodiment, the difluprednate is administered to the eye of the subject for a period of about seven weeks. In a specific embodiment, the difluprednate is administered to the eye of the subject for a period of about twelve weeks. In another specific embodiment, the steroid treatment comprises administering difluprednate once on the first day of the steroid treatment, followed by four times daily for about four weeks, followed by three times daily for about one week, followed by two times daily for about one week, followed by one time daily for about one week. In yet another specific embodiment, the steroid treatment consists of administering difluprednate four times daily for three days prior to surabgene lomparvovec treatment, followed by four times daily for about four weeks, followed by three times daily for about one week, followed by two times daily for about one week, followed by one time daily for about one week. In certain embodiments, the difluprednate is administered in the form of an ophthalmic emulsion. In certain embodiments, the ophthalmic emulsion comprises 0.5 mg/mL (0.05%) difluprednate. In certain embodiments, each administration of difluprednate comprises instilling one drop of the ophthalmic emulsion in the eye of the subject. In certain embodiments, each administration of difluprednate consists of instilling one drop of the ophthalmic emulsion in the eye of the subject.

In certain embodiments of the method of treating DR with or without CI-DME provided herein, the treatment comprises administering surabgene lomparvovec to the suprachoroidal space of an eye of the subject; and the steroid treatment comprises administering prednisolone to the eye of the subject. In certain embodiments, the prednisolone is administered daily to the eye of the subject. In certain embodiments, the steroid treatment comprises administering prednisolone four times daily. In certain embodiments, the prednisolone is administered four times daily for at least one week, at least two weeks, at least three weeks, or at least four weeks. In a specific embodiment, the prednisolone is administered four times daily for about four weeks. In certain embodiments, the steroid treatment comprises administering prednisolone three times daily. In certain embodiments, the prednisolone is administered three times daily for at least one day, at least two days, at least three days, at least four days, at least five days, at least six days, or at least one week. In a specific embodiment, the prednisolone is administered three times daily for about one week. In certain embodiments, the steroid treatment comprises administering prednisolone two times daily. In certain embodiments, the prednisolone is administered two times daily for at least one day, at least two days, at least three days, at least four days, at least five days, at least six days, or at least one week. In a specific embodiment, the prednisolone is administered two times daily for about one week. In certain embodiments, the steroid treatment comprises administering prednisolone one time daily. In certain embodiments, the prednisolone is administered one time daily for at least one day, at least two days, at least three days, at least four days, at least five days, at least six days, or at least one week. In a specific embodiment, the prednisolone is administered one time daily for about one week. In certain embodiments, the prednisolone is administered to the eye of the subject for a period of at least one week, at least two weeks, at least three weeks, at least four weeks, at least five weeks, at least six weeks, or at least seven weeks. In certain embodiments, the prednisolone is administered to the eye of the subject for a period of at least one week, at least two weeks, at least three weeks, at least four weeks, at least five weeks, at least six weeks, at least seven weeks, at least eight weeks, at least nine weeks, at least ten weeks, at least eleven weeks, at least twelve weeks, at least thirteen weeks, at least fourteen weeks, at least fifteen weeks, at least sixteen weeks, at least seventeen weeks, at least eighteen weeks, at least nineteen weeks or at least twenty weeks. In a specific embodiment, the prednisolone is administered to the eye of the subject for a period of about seven weeks. In a specific embodiment, the prednisolone is administered to the eye of the subject for a period of about twelve weeks. In another specific embodiment, the steroid treatment comprises administering prednisolone once on the first day of the steroid treatment, followed by four times daily for about four weeks, followed by three times daily for about one week, followed by two times daily for about one week, followed by one time daily for about one week. In yet another specific embodiment, the steroid treatment consists of administering prednisolone four times daily for three days prior to surabgene lomparvovec treatment, followed by four times daily for about four weeks, followed by three times daily for about one week, followed by two times daily for about one week, followed by one time daily for about one week. In certain embodiments, the prednisolone is administered in the form of an ophthalmic suspension. In certain embodiments, the ophthalmic suspension comprises 1% prednisolone acetate. In certain embodiments, each administration of prednisolone comprises instilling two drops of the ophthalmic suspension in the eye of the subject. In certain embodiments, each administration of prednisolone consists of instilling two drops of the ophthalmic suspension in the eye of the subject.

In certain embodiments of the method of treating DR with or without CI-DME provided herein, the treatment comprises administering surabgene lomparvovec to the suprachoroidal space of an eye of the subject; and the steroid treatment comprises administering dexamethasone to the eye of the subject. In certain embodiments, the dexamethasone is administered daily to the eye of the subject. In certain embodiments, the steroid treatment comprises administering dexamethasone four times daily. In certain embodiments, the dexamethasone is administered four times daily for at least one week, at least two weeks, at least three weeks, or at least four weeks. In a specific embodiment, the dexamethasone is administered four times daily for about four weeks. In certain embodiments, the steroid treatment comprises administering dexamethasone three times daily. In certain embodiments, the dexamethasone is administered three times daily for at least one day, at least two days, at least three days, at least four days, at least five days, at least six days, or at least one week. In a specific embodiment, the dexamethasone is administered three times daily for about one week. In certain embodiments, the steroid treatment comprises administering dexamethasone two times daily. In certain embodiments, the dexamethasone is administered two times daily for at least one day, at least two days, at least three days, at least four days, at least five days, at least six days, or at least one week. In a specific embodiment, the dexamethasone is administered two times daily for about one week. In certain embodiments, the steroid treatment comprises administering dexamethasone one time daily. In certain embodiments, the dexamethasone is administered one time daily for at least one day, at least two days, at least three days, at least four days, at least five days, at least six days, or at least one week. In a specific embodiment, the dexamethasone is administered one time daily for about one week. In certain embodiments, the dexamethasone is administered to the eye of the subject for a period of at least one week, at least two weeks, at least three weeks, at least four weeks, at least five weeks, at least six weeks, or at least seven weeks. In certain embodiments, the dexamethasone is administered to the eye of the subject for a period of at least one week, at least two weeks, at least three weeks, at least four weeks, at least five weeks, at least six weeks, at least seven weeks, at least eight weeks, at least nine weeks, at least ten weeks, at least eleven weeks, at least twelve weeks, at least thirteen weeks, at least fourteen weeks, at least fifteen weeks, at least sixteen weeks, at least seventeen weeks, at least eighteen weeks, at least nineteen weeks or at least twenty weeks. In a specific embodiment, the dexamethasone is administered to the eye of the subject for a period of about seven weeks. In a specific embodiment, the dexamethasone is administered to the eye of the subject for a period of about twelve weeks. In another specific embodiment, the steroid treatment comprises administering dexamethasone once on the first day of the steroid treatment, followed by four times daily for about four weeks, followed by three times daily for about one week, followed by two times daily for about one week, followed by one time daily for about one week. In yet another specific embodiment, the steroid treatment consists of administering dexamethasone four times daily for three days prior to surabgene lomparvovec treatment, followed by four times daily for about four weeks, followed by three times daily for about one week, followed by two times daily for about one week, followed by one time daily for about one week. In certain embodiments, the dexamethasone is administered in the form of an ophthalmic solution. In certain embodiments, the ophthalmic solution comprises 0.1% dexamethasone sodium phosphate. In certain embodiments, each administration of dexamethasone comprises instilling two drops of the ophthalmic solution in the eye of the subject. In certain embodiments, each administration of dexamethasone consists of instilling two drops of the ophthalmic solution in the eye of the subject.

In some embodiments of the method of treating DR with or without CI-DME, the steroid treatment enables administration of surabgene lomparvovec at doses equal to or greater than about 1×10¹² GC/eye. In some embodiments of the method of treating DR with or without CI-DME, the steroid treatment enables administration of surabgene lomparvovec at doses equal to or greater than about 5×10¹¹ GC/eye.

In some embodiments of the method of treating DR with or without CI-DME, surabgene lomparvovec is administered at a dose of between about 5×10¹¹ genome copies per eye and about 2×10¹² genome copies per eye. In certain embodiments, surabgene lomparvovec is administered at a dose of about 5×10¹¹ genome copies per eye. In certain embodiments, surabgene lomparvovec is administered at a dose of about 1×10¹² genome copies per eye. In certain embodiments, surabgene lomparvovec is administered at a dose of about 1.5×10¹² genome copies per eye. In certain embodiments, surabgene lomparvovec is administered at a dose of about 2×10¹² genome copies per eye.

In certain embodiments of the method of treating DR with or without CI-DME, surabgene lomparvovec is administered by a single suprachoroidal injection. In certain embodiments, administration of the steroid as described above ameliorates or prevents ocular inflammation. In certain embodiments, administration of the steroid as described above ameliorates or prevents intraocular inflammation. In certain embodiments, administration of the steroid as described above ameliorates or prevents episcleritis. In certain embodiments, administration of the steroid as described above ameliorates or prevents ocular inflammation associated with the dose of surabgene lomparvovec, the number of suprachoroidal injections, and/or the location of suprachoroidal injections. In certain embodiments, administration of the steroid as described above ameliorates or prevents intraocular inflammation associated with the dose of surabgene lomparvovec, the number of suprachoroidal injections, and/or the location of suprachoroidal injections. In certain embodiments, administration of the steroid as described above ameliorates or prevents episcleritis associated with the dose of surabgene lomparvovec, the number of suprachoroidal injections, and/or the location of suprachoroidal injections. In certain embodiments, the steroid treatment comprises administering a therapeutically effective amount of a corticosteroid. In certain embodiments, the steroid treatment comprises topically administering a therapeutically effective amount of a steroid, for example, a corticosteroid. In certain embodiments, the corticosteroid is selected from the group consisting of prednisolone, dexamethasone, and difluprednate. In certain embodiments, the corticosteroid is prednisolone. In certain embodiments, the corticosteroid is dexamethasone. In certain embodiments, the corticosteroid is difluprednate.

In certain embodiments of the method of treating DR with or without CI-DME, surabgene lomparvovec is administered at a dose of at least about 5×10¹¹ genome copies per eye. In certain embodiments, surabgene lomparvovec is administered at a dose of between about 5×10¹¹ genome copies per eye and about 2×10¹² genome copies per eye. In certain embodiments, surabgene lomparvovec is administered at a dose of at least about 5×10¹¹ genome copies per eye. In certain embodiments, surabgene lomparvovec is administered at a dose of about 1×10¹² genome copies per eye. In certain embodiments, surabgene lomparvovec is administered at a dose of about 1.5×10¹² genome copies per eye. In certain embodiments, surabgene lomparvovec is administered at a dose of about 2×10¹² genome copies per eye. In certain embodiments, surabgene lomparvovec is administered by a single suprachoroidal injection. In certain embodiments, topical administration of the steroid as described above ameliorates or prevents ocular inflammation. In certain embodiments, topical administration of the steroid as described above ameliorates or prevents intraocular inflammation. In certain embodiments, topical administration of the steroid as described above ameliorates or prevents episcleritis. In certain embodiments, topical administration of the steroid as described above prevents ocular inflammation. In certain embodiments, topical administration of the steroid as described above prevents intraocular inflammation. In certain embodiments, topical administration of the steroid prevents episcleritis. In certain embodiments, topical administration of the steroid as described above ameliorates or prevents ocular inflammation associated with the dose of surabgene lomparvovec, the number of suprachoroidal injections, and/or the location of suprachoroidal injections. In certain embodiments, topical administration of the steroid as described above ameliorates or prevents intraocular inflammation associated with the dose of surabgene lomparvovec, the number of suprachoroidal injections, and/or the location of suprachoroidal injections. In certain embodiments, topical administration of the steroid as described above ameliorates or prevents episcleritis associated with the dose of surabgene lomparvovec, the number of suprachoroidal injections, and/or the location of suprachoroidal injections.

In certain embodiments of the method of treating DR with or without CI-DME, the steroid treatment comprises topically administering a therapeutically effective amount of a corticosteroid. In certain embodiments, the topical corticosteroid is selected from the group consisting of prednisolone, dexamethasone, and difluprednate. In certain embodiments, the topical corticosteroid is prednisolone. In certain embodiments, the topical corticosteroid is dexamethasone. In certain embodiments, the topical corticosteroid is difluprednate.

In some embodiments of the method of treating DR with or without CI-DME, the method results in an efficacy result, a safety result, or both.

In some embodiments of the method of treating DR with or without CI-DME, the efficacy result is selected from the group consisting of (1) the subject maintaining best-corrected visual acuity (BCVA) relative to baseline, (2) the subject showing an improvement in their diabetic retinopathy severity scale (DRSS) score relative to baseline, (3) the subject no longer having CI-DME that was present when the surabgene lomparvovec was administered, (4) the subject not developing any sight-threatening ocular diabetic complications in the eye treated with surabgene lomparvovec, (5) the subject not developing ocular diabetic complications requiring treatment per standard of care (SOC) for such complications in the eye treated with surabgene lomparvovec, (6) if the subject develops neovascularization due to diabetic retinopathy requiring treatment per standard of care (SOC), then the subject only requires one SOC treatment to treat the neovascularization, (7) the subject not developing ocular diabetic complications requiring surgical intervention per standard of care (SOC) for such complications in the eye treated with surabgene lomparvovec, (8) the subject has a CST of less than 320 μm or no more than 290 μm after administration of the surabgene lomparvovec, (9) the subject not showing a worsening in their diabetic retinopathy severity scale (DRSS) score of two steps or more relative to baseline, (10) the subject not developing a vision-threatening complication (VTC) due to diabetic retinopathy, (11) subject having either a one-step reduction in their DRSS score, no change in their DRSS score, or a one-step improvement in their ETDRS DRSS score, (12) the subject maintains a ETDRS BCVA of within ±5 letters at week 48 after administration of the surabgene lomparvovec when compared to their ETDRS BCVA when the surabgene lomparvovec was administered, (13) the subject gains more than 5 letters of ETDRS BCVA at week 48 after administration of the surabgene lomparvovec when compared to their ETDRS BCVA when the surabgene lomparvovec was administered. In some embodiments, the baseline is measured and/or determined 1, 2, 3, 4, 5, 6, or 7 days before the surabgene lomparvovec is administered. In some embodiments, the baseline is measured and/or determined 1, 2, 3, 4, 5, 6, 7, 8, 9, or 10 weeks before the surabgene lomparvovec is administered.

In some embodiments of the method of treating DR with or without CI-DME, the efficacy results are seen within or at 12, 14, 28, 30, 38, 48, 54, 96, or 144 weeks after the administration of the surabgene lomparvovec. In some embodiments, the efficacy result is seen at all or some of those timepoints.

In some embodiments of the method of treating DR with or without CI-DME, the efficacy result is the subject maintaining best-corrected visual acuity (BCVA) relative to baseline. In some embodiments of the method of treating DR with or without CI-DME, the the subject maintains a ETDRS BCVA of 69 or more letters at week 14, 30, 38, or 54 after the surabgene lomparvovec is administered.

In some embodiments of the method of treating DR with or without CI-DME, the efficacy result is the subject showing an improvement in their diabetic retinopathy severity scale (DRSS) score relative to baseline. In some embodiments of the method of treating DR with or without CI-DME, the subject has a 1-step or greater, 2-step or greater, or 3-step or greater improvement in their DRSS score at week 12, 14, 28, 30, 38, 48, 54, 96, or 144 after administration of the surabgene lomparvovec.

In some embodiments of the method of treating DR with or without CI-DME, the efficacy result is the subject no longer having CI-DME that was present when the surabgene lomparvovec was administered. In some embodiments of the method of treating DR with or without CI-DME, the subject has an absence of CI-DME at week 54 after administration of the surabgene lomparvovec.

In some embodiments of the method of treating DR with or without CI-DME, the efficacy result is the subject not developing any sight-threatening ocular diabetic complications in the eye treated with surabgene lomparvovec. In some embodiments of the method of treating DR with or without CI-DME, the subject does not develop any sight-threatening ocular diabetic complications in the eye treated with surabgene lomparvovec over the period of time from the administration of the surabgene lomparvovec to week 28 or 54 after the administration of the surabgene lomparvovec.

In some embodiments of the method of treating DR with or without CI-DME, the efficacy result is the subject not developing ocular diabetic complications requiring treatment per standard of care (SOC) for such complications in the eye treated with surabgene lomparvovec. In some embodiments of the method of treating DR with or without CI-DME, the subject does not develop ocular diabetic complications requiring treatment per standard of care (SOC) for such complications in the eye treated with surabgene lomparvovec over the period of time from the administration of the surabgene lomparvovec to week 28 or 54 after the administration of the surabgene lomparvovec, and wherein the SOC is anti VEGF treatment, PRP treatment, laser treatment, or steroid treatment. In some embodiments of the method of treating DR with or without CI-DME, the ocular diabetic complication is neovascularization due to diabetic retinopathy.

In some embodiments of the method of treating DR with or without CI-DME, the efficacy result is, if the subject develops neovascularization due to diabetic retinopathy requiring treatment per standard of care (SOC), then the subject only requires one SOC treatment to treat the neovascularization. In some embodiments of the method of treating DR with or without CI-DME, the subject develops neovascularization due to diabetic retinopathy requiring treatment per standard of care (SOC) for such complications in the eye treated with surabgene lomparvovec over the period of time from the administration of the surabgene lomparvovec to week 28 or 54 after the administration of the surabgene lomparvovec; wherein the SOC is anti VEGF treatment or PRP treatment, and wherein the subject only receives one SOC treatment. In some embodiments, the SOC is anti-VEGF treatment (e.g., an anti-VEGF agent injection such as ranibizumab or aflibercept). In some embodiments, the SOC is PRP treatment.

In some embodiments of the method of treating DR with or without CI-DME, the efficacy result is the subject not developing ocular diabetic complications requiring surgical intervention per standard of care (SOC) for such complications in the eye treated with surabgene lomparvovec. In some embodiments of the method of treating DR with or without CI-DME, the subject does not develop ocular diabetic complications requiring surgical intervention per standard of care (SOC) for such complications in the eye treated with surabgene lomparvovec over the period of time from the administration of the surabgene lomparvovec to week 54 after the administration of the surabgene lomparvovec. In some embodiments, the diabetic complications requiring surgical intervention per SOC is retinal detachment.

In some embodiments of the method of treating DR with or without CI-DME, the efficacy result is the subject has a CST of less than 320 μm or no more than 290 μm after administration of the surabgene lomparvovec. In some embodiments of the method of treating DR with or without CI-DME, the subject has a CST of less than 320 μm or no more than 290 μm at week 30 or 54 after administration of the surabgene lomparvovec.

In some embodiments of the method of treating DR with or without CI-DME, the efficacy result is the subject not showing a worsening in their diabetic retinopathy severity scale (DRSS) score of two steps or more relative to baseline. In some embodiments of the method of treating DR with or without CI-DME, the subject does not have a two or more step worsening in their ETDRS DRSS score at week 48, 96, or 144 after administration of the surabgene lomparvovec compared to their ETDRS DRSS score when the surabgene lomparvovec was administered.

In some embodiments of the method of treating DR with or without CI-DME, the efficacy result is the subject not developing a vision-threatening complication (VTC) due to diabetic retinopathy. In some embodiments of the method of treating DR with or without CI-DME, the subject does not develop a vision-threatening complication (VTC) due to diabetic retinopathy by week 48, 96, or 144 after administration of the surabgene lomparvovec. In some embodiments, the VTC is CI-DME. In some embodiments, the VTC is proliferative diabetic retinopathy (PDR), wherein the PDR is characterized by one or more of (1) DRSS level increasing to 61 or higher, (2) presence of iris neovascularization of at least 2 cumulative clock hours and/or ANV on gonioscopy, and (2) presence of neovascular glaucoma.

In some embodiments of the method of treating DR with or without CI-DME, the efficacy result is subject having either a one-step reduction in their DRSS score, no change in their DRSS score, or a one-step improvement in their ETDRS DRSS score. In some embodiments of the method of treating DR with or without CI-DME, the subject has a one-step reduction in their ETDRS DRSS score at week 48, 96, or 144 after administration of the surabgene lomparvovec when compared to their ETDRS DRSS score when the surabgene lomparvovec was administered. In some embodiments of the method of treating DR with or without CI-DME, the subject has no change in their ETDRS DRSS score at week 48, 96, or 144 after administration of the surabgene lomparvovec when compared to their ETDRS DRSS score when the surabgene lomparvovec was administered. In some embodiments of the method of treating DR with or without CI-DME, the subject has a one-step improvement in their ETDRS DRSS score at week 48, 96, or 144 after administration of the surabgene lomparvovec when compared to their ETDRS DRSS score when the surabgene lomparvovec was administered.

In some embodiments of the method of treating DR with or without CI-DME, the efficacy result is the subject maintains a ETDRS BCVA of within ±5 letters at week 48 after administration of the surabgene lomparvovec when compared to their ETDRS BCVA when the surabgene lomparvovec was administered. In some embodiments of the method of treating DR with or without CI-DME, the subject maintains a ETDRS BCVA of within ±5 letters at week 48, 96, or 104 after administration of the surabgene lomparvovec when compared to their ETDRS BCVA when the surabgene lomparvovec was administered.

In some embodiments of the method of treating DR with or without CI-DME, the efficacy result is the subject gains more than 5 letters of ETDRS BCVA at week 48 after administration of the surabgene lomparvovec when compared to their ETDRS BCVA when the surabgene lomparvovec was administered. In some embodiments of the method of treating DR with or without CI-DME, the subject gains more than 5 letters of ETDRS BCVA at week 48 after administration of the surabgene lomparvovec when compared to their ETDRS BCVA when the surabgene lomparvovec was administered.

In some embodiments of the method of treating DR with or without CI-DME, the safety result is mitigation of ocular inflammation of episcleritis, anterior chamber cell, vitritis, iridocyclitis, iritis, eye inflammation, anterior chamber flare, vitreal cells, keratic precipitates, and uveitis.

In some embodiments of the method of treating DR with or without CI-DME, the safety result may be seen at the same time points as the efficacy results (e.g., through and/or week 12, 14, 28, 30, 38, 48, 54, 96, or 144 after the surabgene lomparvovec administration). However, in some embodiments of the method of treating DR with or without CI-DME, the safety result may be seen much sooner than the efficacy results.

Thus, in some embodiments of the method of treating DR with or without CI-DME, the safety result can be observed through and/or at 1, 2, 3, 4, 5, 6, or 7 days after administration of the surabgene lomparvovec. In some embodiments of the method to treat DR with or without CI-DME, the safety result can be observed through and/or at 1, 2, 3, or 4 weeks after the administration of the surabgene lomparvovec.

In some embodiments of the method of treating DR with or without CI-DME, the safety effect can be a reduced amount of inflammation (e.g., less episcleritis, anterior chamber cell, vitritis, iridocyclitis, iritis, eye inflammation, anterior chamber flare, vitreal cells, keratic precipitates, and/or uveitis) in a proportion of subjects when compared to a reference population that received concomitant steroids as described in the Sampling and Monitoring Efficiency section above. Accordingly, a subject can have a reduction in the probability of experiencing inflammation (e.g., lower chance of episcleritis, anterior chamber cell, vitritis, iridocyclitis, iritis, eye inflammation, anterior chamber flare, vitreal cells, keratic precipitates, and/or uveitis) that is equivalent and/or proportional to the difference seen between the proportion of subjects treated by the method of treating DR with or without CI-DME described herein and the proportion of subjects in the reference population.

In some embodiments of the method of treating DR with or without CI-DME, the safety effect can be a complete lack of inflammation (e.g., no episcleritis, anterior chamber cell, vitritis, iridocyclitis, iritis, eye inflammation, anterior chamber flare, vitreal cells, keratic precipitates, and/or uveitis) when compared to subjects in the reference population as described in the Sampling and Monitoring Efficiency section above.

In some embodiments of the method of treating DR with or without CI-DME, the subjects can be screened for responsiveness to ranibizumab prior to the administration of the surabgene lomparvovec by being administered one or more screening injections of ranibizumab prior to the administration of the surabgene lomparvovec and observing the effect of the screening injection on the subject.

In some embodiments of the method of treating DR with or without CI-DME, the screening ranibizumab injection can be administered about 10 weeks prior to administration of the surabgene lomparvovec, and the biological effect can be observed at some time after the screening ranibizumab injection. In some embodiments of the method of treating DR with or without CI-DME, the observation of the biological effect can be about 1 week after the screening ranibizumab injection is administered. In other embodiments, the screening ranibizumab can be administered 0 weeks prior to the administration of the surabgene lomparvovec (e.g., in cases where it is decided to administer the surabgene lomparvovec to the subject the same day or week they are screened for responsiveness to the screening ranibizumab injection). In other embodiments, the ranibizumab can be administered more than 10 weeks (e.g., 10, 11, 12, or more weeks) or less than 10 weeks (e.g., 9, 8, 7, 6, 5, 4, 3, 2, or 1 week) before the surabgene lomparvovec, although the subject may require conventional anti-VEGF treatment for the DR with or without CI-DME (e.g., via injections of ranibizumab, aflibercept, or other antiVEGF agent per the label) if the screening is done more than 10 weeks prior to the eventual surabgene lomparvovec administration due to the need for some form of treatment until the surabgene lomparvovec can be administered and take effect.

The biological effect to be observed as evidence of the subject's responsiveness can be any biological effect known by a person skilled in the art to be associated with anti-VEGF agents like ranibizumab when used to treat DR with or without CI-DME, such as central subfield thickness (CST). Additional biological effects associated with anti-VEGF agents like ranibizumab when used to treat DR with or without CI-DME would be identifiable by a person skilled in the art.

Accordingly, in some embodiments of the method of treating DR with or without CI-DME, the biological effect can be the subject showing a reduction in central subfield thickness (CST) of at least 10% when compared to the CST when the screening ranibizumab injection was administered.

In some embodiments of the method of treating DR with or without CI-DME, the subject may not require any screening injection of ranibizumab because the patient may have previously shown the necessary responsiveness to ranibizumab (e.g., the patient has already been receiving a successful treatment regimen of ranibizumab prior to consideration for treatment with surabgene lomparvovec).

Once the subject's responsiveness to the ranibizumab protein is confirmed, the surabgene lomparvovec can be administered (e.g., about 0, 1, 2, 3, 4, 5, 6, 7, 8, 9, 10, 11, or 12 weeks or more after the administration of the screening ranibizumab injection). However, in some embodiments, an anti-VEGF (e.g., ranibizumab or aflibercept) pre-treatment injection can be administered in between the screening ranibizumab injection and the administration of the surabgene lomparvovec. In Some embodiments, another anti-VEGF (e.g., ranibizumab or aflibercept) pre-treatment injection can be administered in between the first anti-VEGF pre-treatment injection and the administration of the surabgene lomparvovec.

Thus, in some embodiments of the method of treating DR with or without CI-DME, the subject receives one or more pre-treatment injections of an anti-VEGF agent such as aflibercept after the screening ranibizumab injection and before the surabgene lomparvovec administration. In some embodiments, the subject receives two pre-treatment injections of aflibercept prior to the administration of the surabgene lomparvovec. In some embodiments, the first of the two pre-treatment aflibercept injections is administered about 4 weeks after the screening ranibizumab injection is administered and about 6 weeks before the surabgene lomparvovec is administered. In some embodiments, the second of the two pre-treatment aflibercept (or other anti-VEGF agent) injections is administered about 4 weeks after the first pre-treatment aflibercept injection is administered and about 2 weeks before the surabgene lomparvovec is administered. These two additional aflibercept injections after the screening injection is useful because it may take some time for the surabgene lomparvovec to transduce retinal cells to be able to produce therapeutically effective amounts of the transgene anti VEGF product, and in situations where the surabgene lomparvovec won't be administered immediately when the subject's responsiveness is confirmed, the patient will often still require treatment of the DR with or without CI-DME until the surabgene lomparvovec is administered. These additional doses of aflibercept can be administered a period of time after the injection of the screening ranibizumab injection (e.g., 4 weeks) as per the label of aflibercept (e.g., the current label for aflibercept indicates that 5 monthly—i.e., every 4 weeks—injections should initially be administered to treat DME or DR; the screening aflibercept (counting the screening ranibizumab as if it were the first of the 5 aflibercept injections) along with the first and second aflibercept injections would thus be the first three of the 5 initial injections per the aflibercept label). The number of weeks between the first pre-treatment aflibercept injection and the administration of the surabgene lomparvovec, as well as the number of weeks between second pre-treatment aflibercept injection and the administration of surabgene lomparvovec would be determined by the number of weeks between the screening ranibizumab injection and the administration of surabgene lomparvovec (e.g., if the 4 weeks between the 5 initial aflibercept injections from the aflibercept label is being followed and the surabgene lomparvovec is administered 9 weeks after the screening ranibizumab injection instead of 10 weeks before the administration of the surabgene lomparvovec with the first pre-treatment aflibercept injection administered 4 weeks after the screening ranibizumab injection and the second pre-treatment aflibercept injection administered 4 weeks after the first, then the first aflibercept injection will be administered 5 weeks before the surabgene lomparvovec—but still 4 weeks after the screening ranibizumab injection-instead of 6 weeks before the surabgene lomparvovec injection, and the second pre-treatment aflibercept injection will be administered 1 week before the surabgene lomparvovec administration instead of two weeks before the surabgene lomparvovec administration). In cases where the surabgene lomparvovec is administered less than 8 weeks after the screening ranibizumab injection, then only one pre-treatment aflibercept injection is administered and the second aflibercept injection after that becomes the first post-treatment aflibercept injection as described below. In cases where the surabgene lomparvovec is administered less than 4 weeks after the screening injection, then both the following two aflibercept injections would become post-treatment aflibercept injections as described below. In cases where the surabgene lomparvovec is administered more than 12 weeks before the surabgene lomparvovec, then a third (or more) pre-treatment aflibercept injection can also be administered before administration of the surabgene lomparvovec. In addition, in situations where the ranibizumab is being administered at times indicated on the label that are different that every 4 weeks (e.g., in accordance with the current aflibercept label, every 2 months after 5 initial monthly injections when the patient has already been on ranibizumab therapy prior to screening), then the period of time between any post-screening ranibizumab injections would be determined by that period of time indicated on the label.

In some embodiments of the method of treating DR with or without CI DME, a subject can also optionally receive one or more anti-VEGF post-treatment injections (e.g., ranibizumab or aflibercept) specifically as part of the treatment after administration of the surabgene lomparvovec, for example to provide treatment of the DR while the surabgene lomparvovec is transducing the retinal cells to produce the ranibizumab-like transgene protein product. In some embodiments, the subject receives one anti-VEGF post-treatment injection (e.g., ranibizumab or aflibercept). In some embodiments, the subject receives two anti-VEGF post-treatment injections (e.g., ranibizumab or aflibercept).

In some embodiments of the method of treating DR with or without CI-DME, the subject receives two post-treatment aflibercept (or other anti-VEGF agent) injections, wherein the first of the two post-treatment aflibercept (or other anti-VEGF agent) injections is administered about 4 weeks after the second of the two pre-treatment aflibercept injections administered between the screening ranibizumab injection and the surabgene lomparvovec, and wherein the second of the two post-treatment aflibercept (or other anti-VEGF agent) injections is administered about 4 weeks after the first post-treatment aflibercept (or other anti-VEGF agent) injection is administered. In this situation, the 4 weeks between each of the 5 injections of the screening ranibizumab injection, the two pre-treatment aflibercept injections, and the two post-treatment aflibercept injections would be consistent with the 5 initial monthly aflibercept injections indicated by the aflibercept label. Other periods of time for the post-treatment aflibercept injections indicated by the aflibercept label are also possible as they were for the pre-treatment aflibercept injection (e.g., the 8 weeks indicated in the aflibercept label). In addition, more than two post-treatment aflibercept (or other anti-VEGF agent) injections can be administered (consistent with their label). For example, if the subject is administered the surabgene lomparvovec the day of the screening for ranibizumab responsiveness (i.e., the week after the screening ranibizumab injection is administered), then the subject will likely ne up to 5 or more post-treatment aflibercept injections (or other anti-VEGF agent) administered with a timing indicated by the label of the aflibercept (or other anti-VEGF agent).

In some embodiments of the method of treating DR with or without CI-DME, the subject is phakic, at least in the eye to which the surabgene lomparvovec is administered. In other embodiments of the method of treating DR with or without CI-DME, the subject is pseudophakic, at least in the eye to which the surabgene lomparvovec is administered.

In some embodiments, the methods described herein are useful for treating a retinal disease associated with hVEGF in a subject in need thereof. In some embodiments, the method for treating a retinal disease with hVEGF in a subject in need thereof results in an efficacy result, a safety result or both, wherein the efficacy result is selected from the group consisting of (1) the subject maintaining best-corrected visual acuity (BCVA) relative to baseline, (2) the subject showing an improvement in their diabetic retinopathy severity scale (DRSS) score relative to baseline, (3) the subject no longer having CI-DME that was present when the surabgene lomparvovec was administered, (4) the subject not developing any sight-threatening ocular diabetic complications in the eye treated with surabgene lomparvovec, (5) the subject not developing ocular diabetic complications requiring treatment per standard of care (SOC) for such complications in the eye treated with surabgene lomparvovec, (6) if the subject develops neovascularization due to diabetic retinopathy requiring treatment per standard of care (SOC), then the subject only requires one SOC treatment to treat the neovascularization, (7) the subject not developing ocular diabetic complications requiring surgical intervention per standard of care (SOC) for such complications in the eye treated with surabgene lomparvovec, (8) the subject has a CST of less than 320 μm or no more than 290 μm after administration of the surabgene lomparvovec, (9) the subject not showing a worsening in their diabetic retinopathy severity scale (DRSS) score of two steps or more relative to baseline, (10) the subject not developing a vision-threatening complication (VTC) due to diabetic retinopathy, (11) subject having either a one-step reduction in their DRSS score, no change in their DRSS score, or a one-step improvement in their ETDRS DRSS score, (12) the subject maintains a ETDRS BCVA of within ±5 letters at week 48 after administration of the surabgene lomparvovec when compared to their ETDRS BCVA when the surabgene lomparvovec was administered, and (13) the subject gains more than 5 letters of ETDRS BCVA at week 48 after administration of the surabgene lomparvovec when compared to their ETDRS BCVA when the surabgene lomparvovec was administered; and wherein the safety result is mitigation of ocular inflammation of episcleritis, anterior chamber cell, vitritis, iridocyclitis, iritis, eye inflammation, anterior chamber flare, vitreal cells, keratic precipitates, and uveitis.

In certain embodiments of the present methods, the method results in prevention of ocular inflammation. In certain embodiments, the method results in ocular inflammation prevented at one or more time points after administration of surabgene lomparvovec. In certain embodiments, the one or more time points after administration of surabgene lomparvovec are selected from day 2; week 1; month 1; month 3; month 6; month 9; year 1; year 1.5; and year 2. In certain embodiments, the method results in the ocular inflammation prevented at each of day 2; week 1; and month 1; optionally month 3; month 6; at month 9; year 1; year 1.5; and year 2.

In certain embodiments of the present methods, the method results in prevention of ocular inflammation and wherein the ocular inflammation of anterior chamber cells, flare, or pigmented cells is prevented. In certain embodiments, the ocular inflammation of vitreous cells, flare, or pigmented cells is prevented. In certain embodiments, wherein the ocular inflammation of vitreous cells, flare, or pigmented cells is prevented.

In certain embodiments of the present methods, the method results in the mitigation of pigmentary changes or cells, vasculitis, occlusion, hypotony, or chorioretinitis. In certain embodiments, the method results in the prevention of pigmentary changes or cells, vasculitis, occlusion, hypotony, or chorioretinitis. In certain embodiments, the method results in an efficacy result that is the subject having either no change in their DRSS score, or a one-step improvement in their DRSS score, or a two-step improvement in their DRSS score, or a three-step improvement in their DRSS score, or a four-step improvement in their DRSS score. In certain embodiments, the method results in the subject having a ≥2 step improvement in their DRSS score at year 1 or at year 2 after administration of the surabgene lomparvovec when compared to their DRSS score when the surabgene lomparvovec was administered.

In certain embodiments of the method for treating a retinal disease associated with hVEGF in a subject in need thereof, the method results in an efficacy result that is the subject showing a reduction in a supplemental anti-VEGF injection annualized rate after the administration of the surabgene lomparvovec. In certain embodiments, the method results in the subject showing a reduction of at least 50% or 75% in a supplemental anti-VEGF injection annualized rate in year 1, or in years 1 and 2, after the surabgene lomparvovec administration when compared with the number of anti-VEGF injections during the year preceding the surabgene lomparvovec administration, and wherein the subject received at least one anti-VEGF injection during the year preceding the surabgene lomparvovec administration. In certain embodiments of the method for treating a retinal disease associated with hVEGF in a subject in need thereof, wherein the subject has received one or more injections of ranibizumab prior to the administration of the surabgene lomparvovec. In certain embodiments of the method, wherein the subject has received one or more injections of aflibercept prior to the administration of the surabgene lomparvovec.

In certain embodiments of the present methods, the subject receives no more than 2, no more than 1, or no supplemental anti-VEGF injections in year 1, or in years 1 and 2, after the administration of the surabgene lomparvovec. In certain embodiments of the present methods, the subject receives no supplemental anti-VEGF injections in year 1, or in years 1 and 2, after the administration of the surabgene lomparvovec. In certain embodiments of the method, wherein the subject has a ≥2 step improvement in their DRSS score.

In certain embodiments of the method for treating a retinal disease associated with hVEGF in a subject in need thereof, wherein the method results in the mitigation of vision threatening complications due to diabetic retinopathy in year 1, or in years 1 and 2, after the administration of the surabgene lomparvovec. In certain embodiments, where in the method results in the mitigation of proliferative diabetic retinopathy (PDR), anterior segment neovascularization (ASNV) or center-involved diabetic macular edema (CI-DME). In certain embodiments, wherein the method results in the mitigation is after the administration of a single dose of the surabgene lomparvovec.

In certain embodiments of the method for treating a retinal disease associated with hVEGF in a subject in need thereof, wherein the subject has received one or more injections of ranibizumab prior to the administration of the surabgene lomparvovec. In certain embodiments of the method, wherein one of the ranibizumab injections is a screening ranibizumab injection administered to the subject about 10 weeks before the administration of the surabgene lomparvovec. In certain embodiments of the method, about one week after the administration of the screening ranibizumab injection, the subject shows a reduction in central subfield thickness (CST) of at least 10% when compared to the CST when the screening ranibizumab injection was administered. In certain embodiments of the method, the subject receives one or two pre-treatment injections of aflibercept prior to the administration of the surabgene lomparvovec. In certain embodiments of the method, the subject receives two pre-treatment injections of aflibercept prior to the administration of the surabgene lomparvovec.

In certain embodiments of the method for treating a retinal disease associated with hVEGF in a subject in need thereof, the first of the two pre-treatment aflibercept injections is administered about 4 weeks after the screening ranibizumab injection is administered and about 6 weeks before the surabgene lomparvovec is administered. In certain embodiments, the second of the two pre-treatment aflibercept injections is administered about 4 weeks after the first pre-treatment aflibercept injection is administered and about 2 weeks before the surabgene lomparvovec is administered. In certain embodiments, the surabgene lomparvovec is administered about 2 weeks after the second pre-treatment aflibercept injection. In certain embodiments, the subject receives one or two post-treatment aflibercept injections after the administration of the surabgene lomparvovec. In certain embodiments, the subject receives two post-treatment aflibercept injections after the administration of the surabgene lomparvovec. In certain embodiments, the first of the two post-treatment aflibercept injections is administered about two weeks after the administration of the surabgene lomparvovec. In certain embodiments, the second of the two post-treatment aflibercept injections is administered about 4 weeks after the first post-treatment aflibercept injection is administered.

In certain embodiments of the method for treating a retinal disease associated with hVEGF in a subject in need thereof, the method results in an efficacy result that is the subject maintaining best-corrected visual acuity (BCVA) relative to baseline. In certain embodiments, the subject maintains a ETDRS BCVA of 69 or more letters at week 14, 30, 38, or 54 after the surabgene lomparvovec is administered. In certain embodiments, the method results in a safety result that is mitigation of ocular inflammation of episcleritis, anterior chamber cell, vitritis, iridocyclitis, iritis, eye inflammation, anterior chamber flare, vitreal cells, keratic precipitates, and uveitis.

In certain embodiments of the method for treating a retinal disease associated with hVEGF in a subject in need thereof, the method results in an efficacy result that is the subject showing an improvement in their diabetic retinopathy severity scale (DRSS) score relative to baseline. In certain embodiments, the subject has a 1-step or greater, 2-step or greater, or 3-step or greater improvement in their DRSS score at week 12, 14, 28, 30, 38, 48, 54, 96, or 144 after administration of the surabgene lomparvovec. In certain embodiments, the method results in a safety result that is mitigation of ocular inflammation of episcleritis, anterior chamber cell, vitritis, iridocyclitis, iritis, eye inflammation, anterior chamber flare, vitreal cells, keratic precipitates, and uveitis.

In certain embodiments of the method for treating a retinal disease associated with hVEGF in a subject in need thereof, the method results in an efficacy result that is the subject no longer having CI-DME that was present when the surabgene lomparvovec was administered. In certain embodiments, the subject has an absence of CI-DME at week 54 after administration of the surabgene lomparvovec. In certain embodiments, the method results in a safety result that is mitigation of ocular inflammation of episcleritis, anterior chamber cell, vitritis, iridocyclitis, iritis, eye inflammation, anterior chamber flare, vitreal cells, keratic precipitates, and uveitis.

In certain embodiments of the method for treating a retinal disease associated with hVEGF in a subject in need thereof, the method results in an efficacy result that is the subject not developing any sight-threatening ocular diabetic complications in the eye treated with surabgene lomparvovec. In certain embodiments, the subject does not develop any sight-threatening ocular diabetic complications in the eye treated with surabgene lomparvovec over the period of time from the administration of the surabgene lomparvovec to week 28 or 54 after the administration of the surabgene lomparvovec. In certain embodiments, the method results in a safety result that is mitigation of ocular inflammation of episcleritis, anterior chamber cell, vitritis, iridocyclitis, iritis, eye inflammation, anterior chamber flare, vitreal cells, keratic precipitates, and uveitis.

In certain embodiments of the method for treating a retinal disease associated with hVEGF in a subject in need thereof, the method results in an efficacy result that is the subject not developing ocular diabetic complications requiring treatment per standard of care (SOC) for such complications in the eye treated with surabgene lomparvovec. In certain embodiments, the subject does not develop ocular diabetic complications requiring treatment per standard of care (SOC) for such complications in the eye treated with surabgene lomparvovec over the period of time from the administration of the surabgene lomparvovec to week 28 or 54 after the administration of the surabgene lomparvovec, and wherein the SOC is anti VEGF treatment, PRP treatment, laser treatment, or steroid treatment. In certain embodiments, the ocular diabetic complication is neovascularization due to diabetic retinopathy. In certain embodiments, the method results in a safety result that is mitigation of ocular inflammation of episcleritis, anterior chamber cell, vitritis, iridocyclitis, iritis, eye inflammation, anterior chamber flare, vitreal cells, keratic precipitates, and uveitis.

In certain embodiments of the method for treating a retinal disease associated with hVEGF in a subject in need thereof, the method results in an efficacy result that is, if the subject develops neovascularization due to diabetic retinopathy requiring treatment per standard of care (SOC), then the subject only requires one SOC treatment to treat the neovascularization. In certain embodiments of the method, the subject develops neovascularization due to diabetic retinopathy requiring treatment per standard of care (SOC) for such complications in the eye treated with surabgene lomparvovec over the period of time from the administration of the surabgene lomparvovec to week 28 or 54 after the administration of the surabgene lomparvovec; wherein the SOC is anti VEGF treatment or PRP treatment, and wherein the subject only receives one SOC treatment. In certain embodiments, the method results in a safety result that is mitigation of ocular inflammation of episcleritis, anterior chamber cell, vitritis, iridocyclitis, iritis, eye inflammation, anterior chamber flare, vitreal cells, keratic precipitates, and uveitis.

In certain embodiments of the method for treating a retinal disease associated with hVEGF in a subject in need thereof, the method results in an efficacy result that is the subject not developing ocular diabetic complications requiring surgical intervention per standard of care (SOC) for such complications in the eye treated with surabgene lomparvovec. In certain embodiments of the method, the subject does not develop ocular diabetic complications requiring surgical intervention per standard of care (SOC) for such complications in the eye treated with surabgene lomparvovec over the period of time from the administration of the surabgene lomparvovec to week 54 after the administration of the surabgene lomparvovec. In certain embodiments, the diabetic complications requiring surgical intervention per SOC is retinal detachment. In certain embodiments, the method results in a safety result that is mitigation of ocular inflammation of episcleritis, anterior chamber cell, vitritis, iridocyclitis, iritis, eye inflammation, anterior chamber flare, vitreal cells, keratic precipitates, and uveitis.

In certain embodiments of the method for treating a retinal disease associated with hVEGF in a subject in need thereof, the method results in an efficacy result that is the subject has a CST of less than 320 μm or no more than 290 μm after administration of the surabgene lomparvovec. In certain embodiments of the method, the subject has a CST of less than 320 μm or no more than 290 μm at week 30 or 54 after administration of the surabgene lomparvovec. In certain embodiments, the method results in a safety result that is mitigation of ocular inflammation of episcleritis, anterior chamber cell, vitritis, iridocyclitis, iritis, eye inflammation, anterior chamber flare, vitreal cells, keratic precipitates, and uveitis. In certain embodiments, the method results in an efficacy result that is the subject not showing a worsening in their diabetic retinopathy severity scale (DRSS) score of two steps or more relative to baseline. In certain embodiments of the method, the subject does not have a two or more step worsening in their ETDRS DRSS score at week 48, 96, or 144 after administration of the surabgene lomparvovec compared to their ETDRS DRSS score when the surabgene lomparvovec was administered. In certain embodiments, the method results in a safety result that is mitigation of ocular inflammation of episcleritis, anterior chamber cell, vitritis, iridocyclitis, iritis, eye inflammation, anterior chamber flare, vitreal cells, keratic precipitates, and uveitis.

In certain embodiments of the method for treating a retinal disease associated with hVEGF in a subject in need thereof, the method results in an efficacy result that is the subject not developing a vision-threatening complication (VTC) due to diabetic retinopathy. In certain embodiments, the subject does not develop a vision-threatening complication (VTC) due to diabetic retinopathy by week 48, 96, or 144 after administration of the surabgene lomparvovec. In certain embodiments, the VTC is CI-DME. In certain embodiments, the VTC is proliferative diabetic retinopathy (PDR), wherein the PDR is characterized by one or more of (1) DRSS level increasing to 61 or higher, (2) presence of iris neovascularization of at least 2 cumulative clock hours and/or ANV on gonioscopy, and (2) presence of neovascular glaucoma. In certain embodiments, the method results in a safety result that is mitigation of ocular inflammation of episcleritis, anterior chamber cell, vitritis, iridocyclitis, iritis, eye inflammation, anterior chamber flare, vitreal cells, keratic precipitates, and uveitis.

In certain embodiments of the method for treating a retinal disease associated with hVEGF in a subject in need thereof, the method results in an efficacy result that is the subject having either a one-step reduction in their DRSS score, no change in their DRSS score, or a one-step improvement in their ETDRS DRSS score. In certain embodiments, the subject has either a one-step reduction in their ETDRS DRSS score, no change in their ETDRS DRSS score, or a one-step improvement in their ETDRS DRSS score at week 48, 96, or 144 after administration of the surabgene lomparvovec when compared to their ETDRS DRSS score when the surabgene lomparvovec was administered. In certain embodiments, the method results in a safety result that is mitigation of ocular inflammation of episcleritis, anterior chamber cell, vitritis, iridocyclitis, iritis, eye inflammation, anterior chamber flare, vitreal cells, keratic precipitates, and uveitis.

In certain embodiments of the method for treating a retinal disease associated with hVEGF in a subject in need thereof, the method results in an efficacy result that is the subject maintains a ETDRS BCVA of within ±5 letters at week 48 after administration of the surabgene lomparvovec when compared to their ETDRS BCVA when the surabgene lomparvovec was administered. In certain embodiments, the subject maintains a ETDRS BCVA of within ±5 letters at week 48, 96, or 104 after administration of the surabgene lomparvovec when compared to their ETDRS BCVA when the surabgene lomparvovec was administered. In certain embodiments, the method results in a safety result that is mitigation of ocular inflammation of episcleritis, anterior chamber cell, vitritis, iridocyclitis, iritis, eye inflammation, anterior chamber flare, vitreal cells, keratic precipitates, and uveitis.

In certain embodiments of the method for treating a retinal disease associated with hVEGF in a subject in need thereof, the method results in an efficacy result that is the subject gains more than 5 letters of ETDRS BCVA at week 48 after administration of the surabgene lomparvovec when compared to their ETDRS BCVA when the surabgene lomparvovec was administered. In certain embodiments, the subject gains more than 5 letters of ETDRS BCVA at week 48 after administration of the surabgene lomparvovec when compared to their ETDRS BCVA when the surabgene lomparvovec was administered. In certain embodiments of the method, the subject is phakic. In certain embodiments of the method, the subject is pseudophakic.

### 7. EXAMPLES

### 7.1 Example 1: Updated Phase 2, Randomized, Dose-Escalation, Ranibizumab-Controlled Study to Evaluate the Efficacy, Safety, and Tolerability of Surabgene Lomparvovec Gene Therapy Delivered Via One or Two Suprachoroidal Space (SCS) Injections in Participants with Neovascular Age-Related Macular Degeneration (nAMD) (AAVIATE; M23-411)

### 7.1.1 Introduction

This Example is an update to Example 9 in PCT application publication WO2024073669, which is incorporated by reference in its entirety. In that Example, Cohorts 1-6 were described. Of those cohorts, Cohort 6 was designed to evaluate the efficacy, safety, and tolerability of SCS administration of surabgene lomparvovec (referred to a “Construct II” therein) at a dose of 1.0×10¹² GC/eye (dose level 3, or DL3). In particular, the subjects in one group of patients in that cohort (Group 2) started a 7 week tapering regimen of topical DUREZOL® (or an equivalent steroid) eye drops treatment beginning the day of SCS administration of surabgene lomparvovec (i.e., concomitantly with the surabgene lomparvovec administration, not before it). In contrast, Cohorts 1-5 did not have any specific steroid regimen.

In this Example, a new and currently ongoing cohort (Cohort 7) is described. This cohort differs from Cohort 6 in that (1) a higher dose will be administered and (2) the steroid regimen will be started 3 days prior to the administration of the surabgene lomparvovec.

In particular, Cohort 7 is designed to evaluate the efficacy, safety, and tolerability of SCS administration of surabgene lomparvovec at a higher dose level than previously tested in the study. Five additional ranibizumab control participants from Cohort 7 will be added to the already existing control group (the ranibizumab control arm combined from Cohorts 1 and 2). Similar to Cohort 6, AAV8 neutralizing antibodies (Nabs) will be measured as part of the baseline laboratory testing in Cohort 7, but will no longer be used for screening purposes to determine eligibility and enrollment.

Approximately 25 eligible participants with nAMD will be enrolled into Cohort 7. Eligibility will be determined during the screening period using similar criteria as Cohort 6, based primarily on demonstrated anatomic responsiveness after one of 2 ranibizumab run-in injections (at Week-4 and Day 1) and stricter requirements related to remaining retinal fluid levels. In the interest of safety, additional exclusion criteria are added for Cohort 7 participants.

Initiation of enrollment in Cohort 7 is predicated on completion of dosing of all participants in Cohort 6 and cumulative review of safety data by the IDMC, inclusive of the data from the 2 week post-administration safety visit of the last Cohort 6 participant dosed. Enrollment into Cohort 7 will begin with the randomization of 2 sentinel participants; 1 sentinel participant randomized to surabgene lomparvovec at dose level 4 (1.5×10¹² GC/eye) and the other to ranibizumab control. Following the 2-week post-administration safety visit, a safety committee will review the safety data for the surabgene lomparvovec sentinel participant and, if no SRTs are observed, the remaining participants within Cohort 7 will be randomized to either surabgene lomparvovec dose level 4 (n=19) or ranibizumab control (n=4).

Visits 1 and 2 for Cohort 7 participants will be the same as described for Cohort 6. At Visit 3 (Day 1), participants who continue to meet all entry criteria will receive a second 0.5-mg intravitreal injection of ranibizumab, and will be randomized to surabgene lomparvovec or ranibizumab control; participants not meeting the ranibizumab response criteria will exit the study as screen failures. Participants in Cohort 7 receiving surabgene lomparvovec will have their study intervention administered at the study site during the Week 2 visit, as a single 100-μL SCS injection using the Clearside SCS Microinjector investigational device. All investigators will be trained on the SCS procedure. A detailed description of the procedure can be found in the SCS surabgene lomparvovec administration manual.

surabgene lomparvovec-randomized participants in Cohort 7 will start an approximate 12-week regimen of DUREZOL® or equivalent steroid treatment beginning 3 days prior to SCS surabgene lomparvovec administration (Week 2 visit) and will have 2 visits for postinjection safety (1 day post-procedure and 1 week post-procedure), followed by an additional follow-up visit 2 weeks after surabgene lomparvovec administration. surabgene lomparvovec participants will have a scheduled intravitreal ranibizumab injection at Week 4 (2 weeks following SCS surabgene lomparvovec administration) and beginning at Week 8, may receive supplemental ranibizumab as needed based on prespecified retreatment criteria determined by SD-OCT and BCVA evaluation.

Cohort 7 ranibizumab control arm participants will have their first post-randomization visit at Week 4 and will receive intravitreal ranibizumab 0.5 mg. Thereafter, they will continue to have monthly (about every 28 days) study visits during which they will receive an intravitreal injection of ranibizumab 0.5 mg. Control arm participants will be offered, if eligible, an opportunity to receive surabgene lomparvovec treatment after completion of the study at Week 52. Control participants who cross over to surabgene lomparvovec following Week 52 will receive a single surabgene lomparvovec injection by SCS delivery using the Clearside SCS microinjector investigational device at Week 54 (at dose level 4:1.5×10¹² GC/eye) with a ~12-week regimen of DUREZOL® or equivalent steroid treatment beginning 3 days prior to SCS surabgene lomparvovec administration, and will follow a similar postsurgical visit schedule for assessments as was outlined for participants who were originally randomized to receive surabgene lomparvovec, for 26 weeks through Week 80. Unlike cross-over (CO) participants from earlier cohorts, Cohort 7 CO participants will receive a scheduled ranibizumab injection at Week 56.

Prior to exiting the study at the time of study completion or early discontinuation, Cohort 7 participants who received surabgene lomparvovec either through original randomization or through CO treatment for control participants will be strongly encouraged to participate in a LTFU study for continued safety evaluation. A separate informed consent for the LTFU study will be signed at the time of participation.

### 7.1.2 Objectives and Endpoints

The objectives and endpoint in this updated Example are essentially the same as they were previously, and are described in the table below.

| | | |
|---|---|---|
| Measure | Objectives | Endpoints |

Primary

| | | |
|---|---|---|
| Efficacy | To evaluate the mean change in | Mean change from baseline in |
| | BCVA for surabgene | BCVA to Week 40 based on the |
| | lomparvovec compared with | ETDRS score |
| | ranibizumab monthly at Week 40 | |

Secondary

| | | |
|---|---|---|
| Safety | To evaluate the safety and | Incidences of overall and ocular |
| | tolerability of surabgene | AEs and SAEs |
| | lomparvovec | Vector shedding analysis in |
| | | serum, urine, and tears |
| | To evaluate the incidences of | Proportion of participants who |
| | ocular inflammation following | experience ocular inflammation |
| | SCS surabgene lomparvovec | following SCS surabgene |
| | administration | lomparvovec administration |
| Efficacy | To evaluate the effect of | Mean change from baseline in |
| | surabgene lomparvovec on CNV | CNV lesion size and leakage area |
| | lesion growth and leakage as | to Week 52 based on FA |
| | measured by FA | Mean change from Week 52 in CNV |
| | | lesion size and leakage area to |
| | | Week 80 based on FA (CO participants) |
| | To evaluate the effect of | Mean change from baseline in |
| | surabgene lomparvovec on | BCVA to Week 52 based on the |
| | BCVA | ETDRS score |
| | | Proportion of participants |
| | | (1) gaining ≥15, ≥10, ≥5, or ≥0 |
| | | letters; (2) losing ≥15, ≥10, ≥5, |
| | | or >0 letters (3) maintaining |
| | | vision (not losing ≥15 letters) |
| | | compared with baseline as per |
| | | BCVA at Week 40 and Week 52 |
| | | Mean change from Week 52 in |
| | | BCVA to Week 80 based on the |
| | | ETDRS score (CO participants) |
| | To evaluate the effect of | Mean change from baseline in |
| | surabgene lomparvovec on CRT, | CRT as measured by SD-OCT to |
| | as measured by SD-OCT | Week 40 and Week 52 |
| | | Mean change from Week 52 in |
| | | CRT as measured by SD-OCT to |
| | | Week 80 (CO participants) |
| | To assess the need for | Mean supplemental anti- |
| | supplemental anti-VEGF therapy | VEGF- injection annualized rate |
| | in participants who receive | in the surabgene lomparvovec |
| | surabgene lomparvovec | treatment arms through Week 40 |
| | treatment | and Week 52 |
| | | Proportion of participants who |
| | | have a reduction of ≥50% and ≥75% |
| | | in supplemental anti-VEGF- |
| | | injection annualized rate |
| | | through Week 40 and Week 52 |
| | | compared with the prior 52 weeks |
| | | preceding the first intravitreal |
| | | ranibizumab injection received as |
| | | part of the Screening Period |
| | | (surabgene lomparvovec |
| | | administered participants) |
| | | Proportion of participants with 0, ≤1, |
| | | and ≤2 supplemental injections |
| | | through Week 40 and Week 52 |
| | | Proportion of participants with 0, ≤1, |
| | | and ≤2 supplemental injections |
| | | through Week 80 for CO participants |
| | | Mean percent reduction in |
| | | supplemental anti-VEGF |
| | | injection annualized rate through |
| | | Week 40 and Week 52 compared |
| | | with the prior 52 weeks preceding |
| | | the first ranibizumab injection |
| | | received as part of the Screening |
| | | Period (surabgene lomparvovec |
| | | administered participants) |
| | | Mean supplemental anti-VEGF injection |
| | | annualized rate after Week 54 |
| | | through Week 80 (CO participants) |
| | | Mean percent reduction in |
| | | supplemental anti-VEGF |
| | | injection annualized rate after |
| | | Week 54 through Week 80 |
| | | relative to the prior supplemental |
| | | anti-VEGF injection annualized |
| | | rate after Day 1 through Week 52 |
| | | (CO participants) |
| | | Time from surabgene |
| | | lomparvovec administration to |
| | | first supplemental anti-VEGF |
| | | injection |
| | | Time from surabgene |
| | | lomparvovec administration to |
| | | first supplemental anti-VEGF |
| | | injection in CO participants |
| Pharmacodynamics | To evaluate the concentration of | Aqueous humor surabgene |
| | surabgene lomparvovec TP in | lomparvovec TP concentration |
| | aqueous humor and serum | over time |
| | | Mean change from baseline in |
| | | serum surabgene lomparvovec TP |
| | | concentration over time |
| | | Mean change from Week 54 in |
| | | serum surabgene lomparvovec TP |
| | | concentration over time |
| | | (CO participants) |

Exploratory

| | | |
|---|---|---|
| Efficacy | To evaluate the effect of | Proportion of participants with no |
| | surabgene lomparvovec on fluid | fluid on SD-OCT |
| | accumulation as assessed by SD- | Proportion of participants with |
| | OCT | stable fluid on SD-OCT within |
| | | 30 μm of baseline |
| Safety | To assess changes in visual | Changes in visual field testing |
| | function by visual fields | over time |
| | To evaluate macular atrophy | Incidence of new area of macular |
| | over time, as assessed by FAF | atrophy by FAF (in participants |
| | To assess, in participants with no | with no macular atrophy at |
| | macular atrophy at baseline, the | baseline) |
| | incidence of new areas of | Mean change from baseline in |
| | macular atrophy, as assessed by | area of macular atrophy based on |
| | FAF | FAF at assessed time points |
| Pharmacodynamics | To evaluate the effect of baseline | Association of baseline positive |
| | positive serum AAV8 NAb | serum AAV8 NAb results with |
| | results on the concentration of | aqueous humor and serum |
| | surabgene lomparvovec TP in | surabgene lomparvovec TP |
| | aqueous humor and serum | concentrations |
| Immunogenicity | To evaluate the immunogenicity | Immunogenicity measurements |
| | of surabgene lomparvovec | (AAV8: anti-AAV8 antibodies |
| | | [serum] and ELISpot; surabgene |
| | | lomparvovec TP: anti-surabgene |
| | | lomparvovec TP antibodies and |
| | | ELISpot) |

AAV8 = adeno-associated virus serotype 8;
AE = adverse event;
BCVA = best-corrected visual acuity;
CNV = choroidal neovascularization;
CO = crossover;
CRT = central retinal thickness;
ELISpot = enzyme-linked ImmunoSpot;
ETDRS = Early Treatment Diabetic Retinopathy Study;
FA = fluorescein angiography;
FAF = fundus autofluorescence;
NAb = neutralizing antibody;
SAE = serious adverse event;
SCS = suprachoroidal space;
SD-OCT = spectral domain-optical coherence tomography;
TP = transgene product;
VEGF = vascular endothelial growth factor

### 7.1.3 Study Design

In Cohort 7 participants are enrolled under a similar design and eligibility criteria as Cohort 6, but would be evaluated at a higher surabgene lomparvovec dose level (1.5×10¹² GC/eye) with a 7-week tapering regimen of DUREZOL or equivalent steroid. Cohort 7 would also have a ranibizumab control arm. Protocol Version 9.0 lengthens the DUREZOL or equivalent steroid tapering regimen to begin 3 days prior to surabgene lomparvovec administration for Cohort 7 and associated CO participants and extends to 12 weeks (see Steroid Regimen below).

Approximately 25 participants will be enrolled into Cohort 7, 20 of whom will be randomized to surabgene lomparvovec and 5 of whom will be randomized to ranibizumab control.

Participants in the ranibizumab control arm will be offered, if eligible, an opportunity to receive surabgene lomparvovec treatment after completion of the study at Week 52. Control arm participants who cross over to surabgene lomparvovec following Week 52 (i.e., crossover [CO] participants) will receive surabgene lomparvovec treatment at Week 54

### 7.1.4 Inclusion Criteria

The inclusion criteria for Cohort 7 are identical to Example 9 of WO2024073669 with the following criterion:
A CRT <400 μm at Visit 1, as determined by the CRC. Note that, if the study eye has disease other than fluid contributing to an increase (i.e., PED or SHRM) in CRT, the participant will be enrolled only if the study eye has <50 μm of fluid in the inner retina (parafovea 3 mm), as determined by the CRC.
Response Criterion: Based on the Visit 2 SD-OCT, there must be improvement in fluid relative to Visit 1, in which there is ≤50 μm* of remaining fluid height in the inner retina (parafovea 3 mm) of the study eye or, if the study eye has <50 μm of fluid at Visit 1, then the study eye must have any improvement in fluid. (*Maximum fluid height of 50 μm can be anywhere in the 3 mm region relative to Visit 1)

### 7.1.5 Exclusion Criteria

The exclusion criteria for Cohort 7 are identical to Cohort 6 in Example 9 of WO2024073669 except for the following criterion:
Participants who required >10 anti-VEGF injections in the 12 months prior to Visit 1.
Active or history of glaucoma, steroid-induced ocular hypertension (Cohort 7 only), or ocular hypertension in the study eye, defined as IOP >21 mmHg.
History of intravitreal therapy in the study eye, such as intravitreal steroid injection or IMP, other than an intravitreal therapy for AMD, in the 6 months prior to Visit 1.
   History of intravitreal pegcetacoplan in the study eye in the 30 days prior to Visit 1.
Participants who had prior focal laser therapy or photodynamic therapy (PDT) in the macula.
Polypoidal choroidal vasculopathy (PCV) in the study eye, as determined by the investigator or the CRC (Cohort 7 only).
Any subretinal hemorrhage in the study eye >50% of the total lesion area or within the parafovea (3 mm center of the macula), as determined by the CRC (Cohort 7 only).
Active or history of uveitis (Cohort 7 only).
   7.1.6 Criteria for Participants in the Control Arm Who Cross Over to Surabgene Lomparvovec Treatment after Week 52

### Inclusion Criteria

1 Study eye will be the eye that qualified at randomization.
2. Participant has a CRT <400 μm of subretinal/intraretinal fluid or (in cases where a participant may have nonfluid elevation in the CRT, e.g., pigment epithelial defect)<50 μm of excess fluid, as confirmed by the CRC.
3. Male participants engaged in a sexual relationship with a WOCBP must be willing to use condoms (and their partners to use a highly effective method of contraception) from the day of surabgene lomparvovec administration until 4 weeks after surabgene lomparvovec administration.

### Exclusion Criteria

1 CNV or macular edema in the study eye secondary to any causes other than AMD.
2. New subfoveal fibrosis or atrophy since randomization, as determined by the CRC, or any condition preventing VA improvement in the study eye.
3. Ocular or periocular infection in the study eye that may interfere with the administration of surabgene lomparvovec.
4. Myocardial infarction, cerebrovascular accident, or transient ischemic attacks since randomization.
5 Uncontrolled hypertension (systolic BP >180 mmHg, diastolic BP >100 mmHg) despite maximal medical treatment.
6. Any concomitant treatment that, in the opinion of the investigator, may interfere with the ocular procedure or healing process. Note if BP is brought below 180/100 mmHg and stabilized by antihypertensive treatment, as determined by the investigator and/or primary care physician, the participant can be rescreened for eligibility.
7 History of malignancy or hematologic malignancy that may compromise the immune system requiring chemotherapy and/or radiation since randomization. Localized basal cell carcinoma will be permitted.
8. PCV in the study eye, as determined by the investigator or the CRC (Cohort 7 CO participants).

### 7.1.7 Study Interventions

Eligible participants in Cohort 7 will be randomized to receive surabgene lomparvovec (DL4) or ranibizumab in the study eye. CO participants from Cohort 7 will receive one injection of surabgene lomparvovec DL4 in the study eye.

Information regarding surabgene lomparvovec, ranibizumab, and DUREZOL for Cohort 7 follows.

| | Surabgene Lomparvovec | Ranibizumab | |
|---|---|---|---|
| Intervention | Dose Level (DL) 4 | (LUCENTIS) | DUREZOL ®^{a} |
| Name | (Cohort 7, Cohort 7 COs) | (Cohort 7, Cohort 7 COs) | (Cohort 7, Cohort 7 COs) |
| Type | Gene therapy | Drug (Cohort 7 control | Drug (topical |
| | | treatment arm and run- | corticosteroid) |
| | | in/post surabgene | |
| | | lomparvovec; supplemental | |
| | | treatment [Cohorts 6 and 7 | |
| | | and Cohort 7 COs]) | |
| IMP or | IMP | NIMP/AxMP | NIMP/AxMP |
| NIMP/AxMP | | | |
| Dose | Suspension | Solution | Ophthalmic emulsion |
| Formulation | | | |
| Unit Dose | 1.5 × 10¹³ GC/mL | 10 mg/mL | 0.5 mg/mL (0.05%) |
| Strength | | | |
| Dose Level(s) | 1.5 × 10¹² GC/eye | 0.5 mg (0.05 mL of | 1 drop |
| | delivered via a single | 10 mg/mL solution) once at | (see below for dosing |
| | 100 μL SCS injection | Week −4 and Day 1 (run- | regimen) |
| | (100-μL total volume) | in), Week 4 (Week 56 for | |
| | | Cohort 7 COs) (2 weeks | |
| | | post surabgene | |
| | | lomparvovec treatment), | |
| | | and PRN starting at | |
| | | Week 8 or Week 60 for | |
| | | Cohort 7 COs (surabgene | |
| | | lomparvovec participants' | |
| | | supplemental treatment), or | |
| | | approximately every 28 days | |
| | | (Cohort 7 control arm) | |
| Route of | Suprachoroidal space | Intravitreal injection in | Topical administration of |
| Administration | injection in the study eye | the study eye | 1 drop into the conjunctival |
| | using the Clearside SCS | | sac of the study eye |
| | Microinjector ® | | |
| | investigational device | | |
| Physical | Surabgene lomparvovec | LUCENTIS is supplied as | DUREZOL (difluprednate |
| Description | investigational product is | a preservative-free, sterile | ophthalmic emulsion) |
| | supplied as a frozen, | solution in a single-use | 0.05% is supplied as a |
| | sterile, single-use | container designed to | sterile, preserved, aqueous |
| | suspension of the AAV | deliver 0.05 mL of | topical ophthalmic |
| | vector active ingredient | 10 mg/mL LUCENTIS | emulsion. |
| | (AAV8.CB7.CI.amd42.RBG) | (0.5 mg dose prefilled | |
| | in a formulation buffer. | syringe or vial) aqueous | |
| | The suspension appears | solution. The solution | |
| | clear to opalescent, | appears colorless to pale | |
| | colorless, and free of | yellow. | |
| | visible particulates at room | | |
| | temperature. | | |
| Manufacturer | Advanced BioScience | Genentech, Inc | Alcon ®, a Novartis |
| | Laboratories, Inc | | Company |
| Packaging and | Surabgene lomparvovec | Ranibizumab will be | DUREZOL (difluprednate |
| Labeling | will be supplied as a sterile, | obtained in commercial | ophthalmic emulsion) |
| | single-use suspension in | packaging, either the | 0.05% is a sterile, aqueous |
| | 2-mL Crystal Zenith ® vials | prefilled syringe or single- | topical ophthalmic |
| | sealed with latex-free | use 2-mL glass vial | emulsion supplied in an |
| | rubber stoppers and | designed to deliver | opaque plastic bottle with a |
| | aluminum flip-off seals. | 0.05 mL of 10 mg/mL | controlled drop tip and a |
| | Each vial will be labeled as | ranibizumab solution. | pink cap; 5 mL in an 8-mL |
| | required per country | | bottle |
| | regulatory requirements. | | |

AAV = adeno-associated virus;
AAV8 = adeno-associated virus serotype 8;
AxMP = auxiliary medicinal product;
COs = crossovers;
IMP = investigational medicinal product;
NIMP = noninvestigational medicinal product;
PRN = as needed;
SCS = suprachoroidal space;
USP = United States Pharmacopeia
^{a}An equivalent steroid may be used instead of DUREZOL.

### 7.1.8 Steroid Regimen

Pre-procedure: Beginning in the morning 3 days prior to SCS surabgene lomparvovec administration (i.e., 3 days prior to the Week 2 visit for Cohort 7 surabgene lomparvovec participants or 3 days prior to the Week 54 visit for Cohort 7 CO participants), participants will instill 1 drop of difluprednate ophthalmic emulsion 0.05% (DUREZOL) or equivalent steroid 4 times daily in the study eye for 3 days.

Day-of/Post-procedure: Starting the day of surabgene lomparvovec administration, participants will then initiate a 12-week taper regimen where they will instill drops in their study eye daily, gradually decreasing in a tapered regimen over a total of 12 weeks as follows:
4 times daily for 4 weeks, followed by
3 times daily for 2 weeks, followed by
2 times daily for 2 weeks, followed by
1 time daily for 2 weeks, followed by
1 time every other day for 2 weeks.

Note: The total duration of the per-protocol steroid prophylaxis regimen is 12 weeks+3 days (87 days).

### 7.2 Example 2: Updated Phase 2, Randomized, Controlled, Dose-Escalation Study to Evaluate the Efficacy, Safety, and Tolerability of RGX-314 Gene Therapy Delivered Via a Single Suprachoroidal Space (SCS) Injection in Participants with Diabetic Retinopathy (DR) with and without Center Involved-Diabetic Macular Edema (CI-DME) (ALTITUDE Also Known as M23-414)

This Example provides an overview of an update to Example 12 in PCT application publication WO2024073669, which is incorporated by reference in its entirety.

In this Example, a new and currently ongoing part (Part 2) and cohort (Cohort A) is described. This part and cohort differs from Cohorts 4 and 5 (which, along with Cohorts 1 and 3, are considered “Part 1”) described in WO2024073669 in that (1) these subjects will have CI-DME, (2) a higher dose will be administered, and (3) the steroid regimen will be started 3 days prior to the administration of the surabgene lomparvovec.

### 7.2.1 Objectives and Endpoints

The objectives and endpoint in this updated Example are described in the table below.

| | |
|---|---|
| Objectives | Endpoints |

Primary

| | | |
|---|---|---|
| Efficacy | To evaluate the | Mean change from baseline in BCVA in the |
| | effect of surabgene | study eye at Week 54 |
| | lomparvovec on | |
| | BCVA at Week 54 | |

Secondary

| | | |
|---|---|---|
| Efficacy | To evaluate the | Mean change from baseline in BCVA in the |
| | effect of surabgene | study eye over time |
| | lomparvovec on | Proportion of participants with BCVA of 20/32 |
| | BCVA over time | or better (ETDRS ≥ 69 letters) in the study eye |
| | | over time |
| | To evaluate the | Proportion of participants with a 1-step or |
| | effect of surabgene | greater, a 2-step or greater, or a 3-step or |
| | lomparvovec on DR | greater worsening in DR in the study eye per |
| | (ETDRS-DRSS) | the ETDRS-DRSS on 4-widefield digital |
| | over time | stereoscopic fundus photography at Week 14, |
| | | Week 30, Week 38, and Week 54 |
| | | Proportion of participants achieving a 2-step or |
| | | greater improvement in DR in the study eye |
| | | per the ETDRS-DRSS on 4-widefield digital |
| | | stereoscopic fundus photography at Week 14, |
| | | Week 30, Week 38, and Week 54 |
| | | Proportion of participants achieving a 0-step |
| | | (no change), a 1-step or greater, a 2-step or |
| | | greater, or a 3-step or greater improvement in |
| | | DR in the study eye per the ETDRS-DRSS on |
| | | 4-widefield digital stereoscopic fundus |
| | | photography at Week 66 and Week 82 (CO participants) |
| | | Proportion of participants with a 1-step or |
| | | greater, a 2-step or greater, or a 3-step or |
| | | greater worsening in DR in the study eye per |
| | | the ETDRS-DRSS on 4-widefield digital |
| | | stereoscopic fundus photography at Week 66 |
| | | and Week 82 (CO participants) |
| | | Mean change from baseline in the study eye in |
| | | ETDRS-DRSS severity steps at Week 22, |
| | | Week 38, and Week 54 and (CO participants) |
| | | change from Week 56 at Week 74 and |
| | | Week 82 |
| | To evaluate the | Proportion of participants with an absence of |
| | effect of surabgene | CI-DME in the study eye at Week 54 |
| | lomparvovec on | |
| | CST (as determined | |
| | by SD-OCT measurement) | |
| | at Week 54 | |
| Safety | To assess the safety | Incidences of overall and ocular AEs |
| | and tolerability of | Vector shedding analysis in serum, urine, and |
| | surabgene lomparvovec | tears |
| | To evaluate the | Proportion of participants who experience |
| | incidences of ocular | ocular inflammation in the study eye following |
| | inflammation following | SCS surabgene lomparvovec administration |
| | SCS surabgene | |
| | lomparvovec | |
| | administration | |
| Safety/Efficacy | To evaluate the | Proportion of participants requiring any |
| | need for additional | additional intervention in the study eye for |
| | SOC intervention | ocular diabetic complications to Week 54 and |
| | due to ocular diabetic | (CO participants) Week 82 |
| | complications | Proportion of participants with any |
| | | sight-threatening ocular diabetic complications |
| | | in the study eye to Week 54 and (CO |
| | | participants) Week 82; for this population, the |
| | | following endpoint will be evaluated: |
| | | Duration of time to development of |
| | | sight-threatening ocular conditions from Day 1 |
| | | and (CO participants) Week 56 |
| | | Proportion of participants developing ocular |
| | | diabetic complications in the study eye |
| | | requiring treatment per SOC (anti-VEGF |
| | | treatment, PRP, laser, or steroids) through |
| | | Week 54 and (CO participants) Week 82; for this |
| | | population, the following endpoints will be evaluated: |
| | | Number of treatments received |
| | | Duration of time from study |
| | | intervention (Day 1 and [CO |
| | | participants] Week 56) to first |
| | | treatment per SOC |
| | | Proportion of participants developing ocular |
| | | diabetic complications in the study eye (e.g., |
| | | neovascularization due to DR) requiring |
| | | treatment per SOC (PRP or anti-VEGF |
| | | treatment) through Week 54 and (CO |
| | | participants) Week 82; for this population, the |
| | | following endpoints will be evaluated: |
| | | Duration of time from study intervention |
| | | (Day 1 and [CO participants] Week 56) |
| | | to first treatment |
| | | Proportion of participants requiring more than |
| | | 1 treatment |
| | | Proportion of participants developing ocular |
| | | diabetic complications in the study eye (e.g., |
| | | retinal detachment) requiring surgical |
| | | intervention per SOC through Week 54 and |
| | | (CO participants) Week 82; for this population, |
| | | the following endpoint will be evaluated: |
| | | Duration of time from study intervention |
| | | (Day 1 and [CO participants] Week 56) |
| | | to surgical intervention |
| | To evaluate the | Mean change from baseline in CST in the |
| | effect of surabgene | study eye on SD-OCT at Week 30 and |
| | lomparvovec on | Week 54 |
| | anatomic outcomes | Mean change from Week 54 in CST in the |
| | assessed using SD- | study eye on SD-OCT at Week 82 (CO participants) |
| | OCT in all surabgene | Proportion of participants achieving ≤290 μm |
| | lomparvovec-treated | in CST in the study eye on SD-OCT at |
| | participants | Week 30 and Week 54 |
| Pharmacodynamics | To measure aqueous | Aqueous surabgene lomparvovec TP |
| | and serum surabgene | concentration at assessed time points |
| | lomparvovec TP | Serum surabgene lomparvovec TP |
| | concentrations | concentration at assessed time points |

Exploratory

| | | |
|---|---|---|
| Efficacy/Safety | To evaluate the | Proportion of participants with visual stability |
| | effect of surabgene | (within 5 ETDRS letters or ±5 ETDRS letters) |
| | lomparvovec on | in the study eye from baseline to Week 54 and |
| | vision outcomes | (CO participants) from Week 56 to Week 82 |
| | (BCVA in all | Proportion of participants with vision gain or |
| | surabgene | vision loss >5 ETDRS letters in the study eye |
| | lomparvovec-treated | from baseline to Week 54 and (CO |
| | participants) | participants) from Week 56 to Week 82 |
| | To evaluate the | Proportion of participants with clinically |
| | effect of surabgene | significant changes in visual field in the study |
| | lomparvovec on | eye from baseline to Week 54, as determined |
| | visual field in all | by the investigator |
| | surabgene | |
| | lomparvovec-treated | |
| | participants | |
| Pharmacodynamics | To evaluate the | Association of baseline positive serum AAV8 |
| | effect of baseline | NAb results with aqueous humor and serum |
| | positive serum | surabgene lomparvovec TP concentrations |
| | AAV8 NAb results | |
| | on the concentration | |
| | of surabgene | |
| | lomparvovec TP in | |
| | aqueous humor and | |
| | serum | |
| Immunogenicity | To assess the | Immunogenicity measurements (AAV8: |
| | immunogenicity of | anti-AAV8 antibodies [serum] and ELISpot; |
| | surabgene | surabgene lomparvovec TP: anti-surabgene |
| | lomparvovec | lomparvovec TP antibodies [serum] and ELISpot) |
| PRO | To evaluate visual | Mean change from Week 54 in the |
| Questionnaires | function, general | supplemented NEI VFQ-25 at Week 82 (CO |
| | health-related QoL, | participants) |
| | and patient | Mean change from Week 54 in EQ-5D-5L at |
| | impression of | Week 82 (CO participants) |
| | severity/change | Mean change from Week 54 in EQ VAS score |
| | using PRO | at Week 82 (CO participants) |
| | questionnaires | Proportion of participants with each level of |
| | | severity in overall problems with vision (PGIS |
| | | score [Week 54 and Week 82 for CO participants]) |
| | | Proportion of participants with each level of |
| | | change in problems with vision over time |
| | | (PGIC score [Week 82 for CO participants]) |

AAV8 = adeno-associated virus serotype 8;
AE = adverse event;
BCVA = best-corrected visual acuity;
CI-DME = center involved-diabetic macular edema;
CO = crossover;
CRC = central reading center;
CST = central subfield thickness;
DR = diabetic retinopathy;
DRSS = Diabetic Retinopathy Severity Scale;
ELISpot = enzyme-linked ImmunoSpot;
ETDRS = Early Treatment Diabetic Retinopathy Study;
Nab = neutralizing antibody;
NEI VFQ-25 = National Eye Institute Visual Functioning Questionnaire 25-item Version;
PGIC = Patient Global Impression of Change;
PGIS = Patient Global Impression of Severity;
PRO = patient-reported outcome;
PRP = panretinal photocoagulation;
QoL = quality of life;
SCS = suprachoroidal space;
SD-OCT = spectral domain-optical coherence tomography;
SOC = standard of care;
TP = transgene product;
VAS = Visual Analogue Scale;
VEGF = vascular endothelial growth factor

### 7.2.2 Study Design

Part 2 is a Phase 2, open-label, parallel arm, randomized (2:1) study in participants with DR with CI-DME. Surabgene lomparvovec at dose level 4 (1.5×10¹² GC/eye) will be evaluated in parallel to aflibercept controls. AAV8 NAbs will be measured in participants in Part 2, but serum titer results will not determine eligibility and randomization into the study.

Approximately 30 participants with DR with CI-DME will be randomized into Cohort A of the study. Approximately 20 participants will receive surabgene lomparvovec at dose level 4 (1.5×10¹² GC/eye) and approximately 10 participants will be evaluated as aflibercept controls. Up to approximately 5 additional participants may be enrolled into Cohort A at the sponsor's discretion.

All participants in Part 2 will receive a 0.3-mg intravitreal injection of ranibizumab in the study eye at Week-10. One week later (Week-9), participants' anatomic response on SD-OCT will be evaluated by the CRC based on prespecified response criteria. Participants not meeting the ranibizumab response criteria will exit the study as screen failures. All eligible participants will then receive 2 mg intravitreal injections of aflibercept at Week-6 and Week-2. Participants who are randomized to surabgene lomparvovec will receive dose level 4 (1.5×10¹² GC/eye) on Day 1 of the study. Participants who are randomized to surabgene lomparvovec will have 2 visits for postinjection safety (1 day post procedure and 1 week post procedure). All participants will receive intravitreal injections of aflibercept at Week 2 and again at Week 6, and control participants will continue to receive aflibercept every 8 weeks until Week 46.

Participants in the aflibercept control arm will be offered, if eligible, an opportunity to cross over to surabgene lomparvovec treatment after Week 54. Cross-over (CO) participants will receive a 2 mg intravitreal injection of aflibercept at Week 54, surabgene lomparvovec treatment at Week 56 at dose level 4 (1.5×10¹² GC/eye), a 2 mg intravitreal injection of aflibercept at Week 58, and aflibercept as supplemental anti-VEGF therapy starting at Week 66. All CO participants will attend study visits and be assessed for safety and efficacy in a similar manner as outlined for participants who were originally randomized to receive surabgene lomparvovec at dose level 4, for 26 weeks through Week 82.

Prior to exiting the study at the time of study completion or early discontinuation, those participants who received surabgene lomparvovec either through randomization or CO treatment for control participants will be strongly encouraged to participate in a LTFU study for continued safety evaluation. A separate informed consent for the follow-up study will be signed at that time.

Participants receiving surabgene lomparvovec will have their study intervention administered at the study site during the Day 1 visit (Week 56 for CO participants). surabgene lomparvovec will be given as a single injection by SCS delivery using the Clearside SCS Microinjector investigational device (note that the Treatment Period of the study begins at the time of surabgene lomparvovec administration). All investigators will be trained on the SCS procedure. A detailed description of the procedure can be found in the SCS surabgene lomparvovec administration manuals.

All participants in Cohort A who are randomized to surabgene lomparvovec and control participants who cross over to surabgene lomparvovec treatment will receive a protocol-mandated regimen of DUREZOL® or equivalent steroid treatment in the study eye before and after SCS surabgene lomparvovec administration (See Steroid Regimen below).

Starting at Week 14 (Week 66 for CO participants), all participants, regardless of treatment assignment, who have ocular diabetic complications requiring supplemental anti-VEGF therapy in the study eye due to worsening CI DME or PDR may be administered aflibercept as required according to prespecified retreatment criteria. Starting on Day 2, all participants may receive, at the investigator's discretion, other interventions per SOC (laser, surgery, steroids) if they have ocular diabetic complications warranting treatment.

Enrollment into Cohort A will begin with the randomization of 2 sentinel participants: one sentinel participant will receive surabgene lomparvovec at dose level 4 and one sentinel participant will be an aflibercept control. The surabgene lomparvovec sentinel participant will be observed for safety during the 2-week post-administration period the sponsor will review the 2-week post-administration safety data for the surabgene lomparvovec sentinel participant; if no SRTs are observed, the remaining 28 participants will be randomized to either surabgene lomparvovec dose level 4 (n=19) or aflibercept control (n=9).

The IDMC will review available safety data at specified intervals throughout Part 2 of the study, including after all surabgene lomparvovec participants are enrolled. During any safety review, the IDMC may recommend halting dosing.

For participants who receive surabgene lomparvovec treatment and CO participants, immunogenicity to the vector (as assessed by anti-AAV8 antibodies [serum], anti-surabgene lomparvovec TP antibodies [serum], and ELISpot [whole blood]), and surabgene lomparvovec TP concentrations (aqueous humor and serum) will be assessed throughout the study.

Efficacy will be the primary focus of Part 2 of the study, as assessed through mean change from baseline in BCVA at Week 54.

Patient-Reported Outcomes (PROs) will be collected for aflibercept control participants who cross over to active treatment after Week 54 using the supplemented NEI VFQ-25, EQ-5D-5L, EQ VAS, PGIS, and PGIC.

Participant safety will also be evaluated through the assessment of AEs (including SAEs and AESIs), as well as assessments of clinical laboratory tests (chemistry, hematology, coagulation, and urinalysis), vector shedding (tears, urine, and serum), and ocular examinations and imaging (BCVA, IOP, slit-lamp biomicroscopy, indirect ophthalmoscopy, ultra-widefield FA, ultra widefield Optos FAF, ultra widefield Optos CFP, 4 widefield digital stereoscopic fundus photography, Humphrey visual field or microperimetry, and SD-OCT). Note that AEs will be collected at all study visits. Participants who show evidence of new retinal hypo/hyper pigmentation changes as compared with baseline will be monitored using SD-OCT scans.

Planned safety monitoring of the study participants will be conducted on an ongoing basis, including reviews conducted by the Medical Monitor and routine reviews conducted by AbbVie/REGENXBIO's safety teams.

### 7.2.3 Inclusion Criteria

The inclusion criteria for Part 2, Cohort A are described below.
1. Men or women 25-89 years of age with DR secondary to diabetes mellitus Type 1; or Participants must have a HbA1c <12% (as confirmed by laboratory assessments obtained at Visit 1 [Week-10] or by a documented laboratory report dated within 60 days prior to Visit 1).
2. Best-corrected visual acuity in the study eye of 78 to 25 ETDRS letters (approximate Snellen equivalent 20/32 to 20/320); note: if both eyes are eligible, the study eye must be the participant's worst seeing eye, as determined by the investigator, prior to enrollment.
3. No evidence in the study eye of high-risk PDR characteristics, including the following:
   New vessels within 1-disc area of the optic nerve.
   Vitreous or preretinal hemorrhage associated with less extensive new vessels at the optic disc, or with new vessels elsewhere that are half a disc area or more in size.
   No evidence in the study eye of anterior segment (e.g., iris or angle) neovascularization on clinical examination.
4. Macular thickening secondary to DME involving the center of the fovea, CST on SD OCT (≥325 μm).
5. Based on the SD-OCT image obtained at Week-9, participants must have improvement in fluid relative to Week-10 (their screening visit) per the response criteria defined below:
a. Response criterion: Participants must have a reduction in central subfield thickness of at least 10% at Week-9 relative to the Week-10 as determined by the CRC.
6. Sexually active male participants with female partners of childbearing potential must be willing to use condoms plus a highly effective form of partner contraception from Visit 5 (Day 1, the day of vector administration) until 4 weeks after vector administration.
7. Must be willing and able to comply with all study procedures and be available for the duration of the study.
8. Must be willing and able to provide written, signed informed consent.
9. May be phakic or pseudophakic.

### 7.2.4 Exclusion Criteria

The exclusion criteria for Part 2, Cohort A, is identical to Part 1 exclusion criteria as described in Example 12 of WO202407366 (Cohorts 4-5), except with respect to the following:
Any ocular condition in the study eye other than CI-DME that could require surgical intervention within the 6 months after Visit 1 (vitreous hemorrhage, cataract, retinal traction, epiretinal membrane, etc.) or any condition in the study eye that may, in the opinion of the investigator, increase the risk to the participant, require either medical or surgical intervention during the study to prevent or treat vision loss, or interfere with the study procedures or assessments (e.g., CRVO, BRVO, and CRAO).
Presence of an implant in the study eye at Visit 1 (excluding intraocular lens).
History of intraocular surgery in the study eye within 6 weeks prior to Visit 1.
Participants who required >10 anti-VEGF injections in the study eye in the 12 months prior to Visit 1 must demonstrate a complete fluid response as determined by CRC review of the Visit 1 SD-OCT, defined as complete resolution of fluid.
Diabetic macular edema diagnosis ≥7 years.
   7.2.5 Eligibility Criteria for Participants in the Control Arm Who Cross Over to ABBV-RGX 314 Treatment after Week 54

### Inclusion Criteria

Participants must meet all the following criteria to be eligible for CO. All ocular criteria refer to the study eye:
1. Study eye will be the eye that qualified at randomization.
2. Best-corrected visual acuity in the study eye of 78 to 25 ETDRS letters (approximate Snellen equivalent 20/32 to 20/320).
3 No evidence in the study eye of high-risk PDR characteristics, including the following:
   New vessels within 1-disc area of the optic nerve.
   Vitreous or preretinal hemorrhage associated with less extensive new vessels at the optic disc, or with new vessels elsewhere that are half a disc area or more in size.
   No evidence in the study eye of anterior segment (e.g., iris or angle) neovascularization on clinical examination.
4. Macular thickening secondary to DME involving the center of the fovea, CST on SD OCT (≥325 μm).
5. Sexually active male participants with female partners of childbearing potential must be willing to use condoms plus a highly effective form of partner contraception from the day of vector administration until 4 weeks after vector administration.
6. Must be willing and able to comply with all study procedures and be available for the duration of the study.

### Exclusion Criteria

Participants are excluded from crossing over if any of the following criteria apply:
1. Neovascularization in the study eye from a cause other than DR, per investigator.
2. Any active or history of ocular or periocular infection or ocular inflammation in the study eye.
3. Any ocular condition in the study eye other than CI-DME that could require surgical intervention within the 6 months after Week 56 (vitreous hemorrhage, cataract, retinal traction, epiretinal membrane, etc.) or any condition in the study eye that may, in the opinion of the investigator, increase the risk to the participant, require either medical or surgical intervention during the study to prevent or treat vision loss, or interfere with the study procedures or assessments (e.g., CRVO, BRVO, and CRAO).
4. Myocardial infarction, cerebrovascular accident, or transient ischemic attacks within the 6 months prior to Week 56.
5. Uncontrolled hypertension (systolic BP >180 mmHg, diastolic BP >100 mmHg) despite maximal medical treatment.
6. Any concomitant treatment that, in the opinion of the investigator, may interfere with the ocular procedure or the healing process.
7. History of malignancy with or without therapy or hematologic malignancy that may compromise the immune system requiring chemotherapy and/or radiation since randomization. Localized basal cell carcinoma will be permitted, as will any cancer not being treated with chemotherapy or other therapy that may compromise the immune system.
8. History of chronic renal failure requiring dialysis or kidney transplant.
9. Initiation of intensive insulin treatment (pump or multiple daily injections) within the 6 months prior to Week 56 or plans to do so between Week 56 and Week 82.

### 7.2.6 Study Interventions

Eligible participants in Cohort A will be randomized 2:1 to receive either a single dose of surabgene lomparvovec at dose level 4 (1.5×10¹² GC/eye) or be evaluated as aflibercept controls.

All participants will receive a 0.3 mg intravitreal injection of ranibizumab in the study eye at Week −10 (Visit 1) and 2 mg intravitreal injections of aflibercept in the study eye at Week −6 (Visit 3), Week −2 (Visit 4), Week 2 (Visit 8), and Week 6 (Visit 9). Starting at Week 14 (Visit 10), aflibercept 2 mg will be administered by intravitreal injection every 8 weeks through Week 46. Participants in the control arm who cross over to surabgene lomparvovec will receive 2 mg of aflibercept at Week 54 (2 weeks before surabgene lomparvovec administration) and Week 58 (2 weeks after surabgene lomparvovec administration), and as supplemental anti-VEGF therapy as needed, starting at Week 66.

Eligible participants who cross over to active treatment will receive one injection of surabgene lomparvovec at dose level 4 in the study eye.

Participants randomized to surabgene lomparvovec treatment and those who cross over to surabgene lomparvovec treatment after Week 54 will receive a protocol-mandated regimen of DUREZOL or equivalent steroid treatment in the study eye beginning 3 days before SCS surabgene lomparvovec administration and in a 12-week tapering regimen following SCS surabgene lomparvovec administration.

Information regarding surabgene lomparvovec, ranibizumab, DUREZOL, and aflibercept follows.

| | Surabgene | | | DUREZOL ® | |
|---|---|---|---|---|---|
| | Lomparvovec | Ranibizumab | Aflibercept | or Equivalent | Triamcinolone |
| | Dose Level 4 | (LUCENTIS ®) | (EYLEA ®) | Steroid | Acetonide |
| Type | Gene therapy | Drug | Drug | Drug (topical | Drug (synthetic |
| | (AAV8.CB7.CI. | | | corticosteroid) | glucocorticoid |
| | amd42.RBG) | | | | corticosteroid) |
| Dose | Solution | Solution | Solution | Ophthalmic | Injectable |
| Formulation | | | | emulsion | suspension |
| Unit Dose | 1.5 × 10¹³ | 6 mg/mL | 40 mg/mL | DUREZOL ®: | 40 mg/mL |
| Strength | GC/mL | | | 0.5 mg/mL | |
| | | | | (0.05%) | |
| | | | | Equivalent | |
| | | | | steroid: as | |
| | | | | labelled | |
| Dosage Levels | 1.5 × 10¹² GC/eye | 0.3 mg (0.05 | 2 mg (2 mg/ | DUREZOL ®: | 40 mg (1 mL of |
| | delivered via a | mL of 6 mg/mL | 0.05 mL | 1 drop | 40 mg/mL |
| | single 100 μL | solution) | solution) | (see Steroid | suspension) |
| | SCS injection | | | Regimen for | |
| | (100 μL total | | | dosing regimen) | |
| | volume) | | | Equivalent | |
| | | | | steroid: as | |
| | | | | labelled. | |
| Route of | Suprachoroidal | Intravitreal | Intravitreal | Topical | Subtenon |
| Administration | space injection | injection in the | injection in the | administration | injection |
| | in the study eye | study eye | study eye | into the | |
| | using the | | | conjunctival sac | |
| | Clearside SCS | | | of the study eye | |
| | Microinjector | | | | |
| | investigational | | | | |
| | device | | | | |
| Physical | Surabgene | LUCENTIS ® | EYLEA ® is | DUREZOL ® | Triamcinolone |
| Description | lomparvovec is | is supplied as a | supplied as a | (difluprednate | is supplied as a |
| | supplied as a | preservative- | preservative- | ophthalmic | sterile aqueous |
| | frozen, sterile, | free, sterile | free, sterile, | emulsion) | suspension. |
| | single-use | solution in a | aqueous | 0.05% is | |
| | solution of the | single-use | solution for | supplied as a | |
| | AAV vector | container | intravitreal | sterile, | |
| | active ingredient | designed to | injection in a | preserved, | |
| | (AAV8.CB7.CI. | deliver | single-dose | aqueous topical | |
| | amd42.RBG) in | 0.05 mL of | container | ophthalmic | |
| | a formulation | 6 mg/mL | designed to | emulsion. Or | |
| | buffer. The | LUCENTIS ® | deliver 0.05 mL | equivalent | |
| | solution appears | (0.3-mg dose | (50 μL) of | steroid | |
| | clear to | prefilled | solution | (multiple) | |
| | opalescent, | syringe or vial) | containing 2 mg | | |
| | colorless, and | aqueous | of aflibercept. | | |
| | free of visible | solution. The | The solution | | |
| | particulates at | solution | appears | | |
| | room | appears | colorless to pale | | |
| | temperature. | colorless to | yellow. | | |
| | | pale yellow. | | | |
| Manufacturer | Advanced | Genentech, Inc | Regeneron | Multiple | Multiple |
| | BioScience | | Pharmaceuticals, | | |
| | Laboratories, Inc | | Inc | | |
| Packaging and | Surabgene | Study | Study | DUREZOL | Triamcinolone |
| Labeling | lomparvovec | intervention | intervention will | (difluprednate | acetonide |
| | will be supplied | will be | be obtained in | ophthalmic | injectable |
| | as a sterile, | obtained in | commercial | emulsion) | suspension, |
| | single-use | commercial | packaging, | 0.05% is a | supplied in vials |
| | solution in 2-mL | packaging, | either the | sterile, aqueous | providing 40 mg |
| | Crystal Zenith® | either the | prefilled syringe | topical | triamcinolone |
| | vials sealed with | prefilled | (NDC | ophthalmic | acetonide per |
| | latex-free rubber | syringe (NDC | 61755-005-01) | emulsion | mL. |
| | stoppers and | 50242-082-03) | or single-use | supplied in an | |
| | aluminum flip- | or single-use | glass vial (NDC | opaque plastic | |
| | off seals. Each | 2-mL glass vial | 61755-005-02) | bottle with a | |
| | vial will be | (NDC 50242-0 | designed to | controlled drop | |
| | labeled as | 82-02) | deliver 0.05 mL | tip and a pink | |
| | required per | designed to | of 40 mg/mL | cap; 5 mL in an | |
| | country | deliver | solution. | 8-mL bottle. Or | |
| | regulatory | 0.05 mL of | | equivalent | |
| | requirements. | 6 mg/mL | | steroid | |
| | | ranibizumab | | (multiple) | |
| | | solution. | | | |

### 7.2.7 Steroid Regimens

Pre-procedure: Beginning in the morning 3 days prior to SCS ABBV-RGX-314 administration (i.e., 3 days prior to the Day 1 visit for Cohort A ABBV-RGX-314 participants or 3 days prior to the Week 56 visit for Cohort A CO participants), participants will instill 1 drop of difluprednate ophthalmic emulsion 0.05% (DUREZOL) or equivalent steroid 4 times daily in the study eye for 3 days.

Day-of/Post-procedure: Starting the day of ABBV-RGX-314 administration, participants will initiate a 12-week taper regimen where they will instill drops in their study eye daily, gradually decreasing in a tapered regimen over a total of 12 weeks as follows:
4 times daily for 4 weeks after treatment, followed by
3 times daily for 2 weeks, followed by
2 times daily for 2 weeks, followed by
1 time daily for 2 weeks, followed by
1 time every other day for 2 weeks

Note: An equivalent steroid option to DUREZOL may be provided.

The total duration of the per-protocol steroid prophylaxis regimen is 12 weeks+3 days (87 days).

### 7.2.8 Results

In the above ongoing study of individuals with diabetic retinopathy following either control or surabgene lomparvovec SCS injection, a summary of safety data by dose level has been obtained (Table 4). The percent of individuals associated with ocular TEAEs increased with higher surabgene lomparvovec dose-levels, regardless of steroid regimen. In the control and dose level 1-3 data, the safety data were completed for a year following injection, while the individuals in the crossover data set were SCS injected and are currently being assessed.

TABLE 4
Safety profile in study eye following injections with surabgene lomparvovec.

| | Control | | Dose Level | | Dose Level | | Dose Level | | Crossover | | Crossover |
|---|---|---|---|---|---|---|---|---|---|---|---|
| | (N = 20) | | 1 (N = 15) | | 2 (N = 35) | | 3 (N = 29) | | DL 2 (N = 9) | | DL 3 (N = 9) |
| Dose Amount | — | | 2.5 × 10¹¹ | | 5.0 × 10¹¹ | | 1.0 × 10¹² | | 5.0 × 10¹¹ | | 1.0 × 10¹² |
| | | | GC/eye | | GC/eye | | GC/eye | | GC/eye | | GC/eye |
| Topical Ocular | No ocular | | No ocular | | No ocular | | Started at | | No ocular | | Started at |
| Steroid Regimen | steroids | | steroids | | steroids | | D 1; 7 week | | steroids | | D 1; 7 week |
| | | | | | | | taper | | | | taper |
| Ocular TEAEs | 4 | (20%) | 13 | (86.7%) | 29 | (82.9%) | 26 | (89.7%) | 8 | (88.9%) | 4 (44.4%) |
| Episcleritis** | 0 | (0%) | 1 | (6.7%) | 5 | (14.3%) | 7 | (24.1%) | 2 | (22.2%) | 2 (22.2%) |
| Intraocular | 0 | (0%) | 1 | (6.7%) | 3 | (8.6%) | 2 | (6.9%) | 0 | (0%) | 1 (11.1%) |

Inflammation (IOI)

**Recurrence in 5 participants (one at dose level 1, one at dose level 2, three at dose level 3 of surabgene lomparvovec)

In the Dose Level 3 and Dose Level 4 treatments, both surabgene lomparvovec dose levels and steroid (Durezol®) regimen was changed. The increased dose of surabgene lomparvovec, from 1×10¹² (dose level 3) to 1.5×10¹² GC/eye (dose level 4), would have been expected to show increased ocular TEAEs, and increased inflammation including episcleritis and intraocular inflammation (IOI). However, the earlier Durezol® regimen (i.e., application of Durezol® beginning 3 days prior to SCS administration of surabgene lomparvovec followed by a 12-week tapering postinjection) used in conjunction with dose level 4 SCS injection resulted in fewer ocular TEAEs. There was a surprisingly lower incidence of inflammation than had occurred with lower dose administration of surabgene lomparvovec and with no cases of episcleritis, which was seen with the lower dose level and concomitant administration of steroids (Table 5).

TABLE 5
Dose Level 3 and Dose Level 4 Safety Results.

| | Dose Level 3 | | |
|---|---|---|---|
| | | Crossover: | Dose Level 4 |
| Dose of surabgene | 1 × 10¹² GC/eye | 1 × 10¹² GC/eye | 1.5 × 10¹² GC/eye |
| lomparvovec | (N = 29) | (N = 9) | (N = 13) |
| Topical Steroid | Started at Day 1 with taper to | | Started at −3 Days |
| Regimen | 7 weeks postinjection | | with taper |
| (Durezol ®) | | | to 12 weeks |
| | | | postinjection |
| Ocular TEAEs | 26 (89.7%) | 4 (44.4%) | 2 (15.4%), |
| | | | all Grade 1 |
| Episcleritis | 7 (24.1%) | 2 (22.2%) | 1 (7.7%) |
| Intraocular | 2 (6.9%) | 1 (11.1%) | 1 (7.7%)** |
| Inflammation (IOI) | | | |

*Majority of subjects up to Week 6 (1 subject at Week 30, 2 subjects at Week 14).
**Occurred as ocular hypertension (grade 1) at day 2, proceeded to IOI (Grade 2); at 2 weeks.

### 7.3 Example 3: A Phase 3 Multi Center, Randomized, Double Masked, Sham-Controlled, Study to Evaluate the Safety and Efficacy of Surabgene Lomparvovec Delivered Via a Single Suprachoroidal Space (SCS) Injection in Subjects with Diabetic Retinopathy without Center Involved-Diabetic Macular Edema (CI-DME), Study M23-415 (APEX)

### 7.3.1 Synopsis

This is a planned phase 3 multi center, randomized, double masked, sham-controlled, study to evaluate the safety and efficacy of surabgene lomparvovec delivered via a single suprachoroidal space (SCS) injection in subjects with diabetic retinopathy without center involved-diabetic macular edema (CI-DME).

Primary Objective: To evaluate the efficacy and safety of a SCS administration of surabgene lomparvovec in subjects with DR without CI-DME who have a DRSS Level 47 or 53 at Baseline.

Primary Endpoint: Occurrence of ≥2-step worsening in DRSS level in the study eye at Week 48.

The key secondary objective is to evaluate the development of VTCs in subjects who received surabgene lomparvovec compared to sham.

### Key Secondary Endpoints

1. Development of a VTC due to a DR (i.e., CI-DME, proliferative diabetic retinopathy [PDR]) in the study eye by Week 48.
2. Progression to PDR in the study eye by Week 48.
3. Development of a VTC due to DR (i.e., CI-DME, PDR) in the study eye by Week 96.
4. Progression to PDR in the study eye by Week 96.
5. Development of CI-DME in the study eye by Week 96.
6 Development of a VTC due to a DR (i.e., CI-DME, proliferative diabetic retinopathy [PDR]) in the study eye by Week 144.
7. Progression to PDR in the study eye by Week 144.
8. Development of CI-DME in the study eye by Week 144.

### Safety Endpoints:

1. To assess the safety and tolerability of surabgene lomparvovec.
2. To evaluate the incidence of ocular inflammation following SCS surabgene lomparvovec administration.

Approximately 400 subjects with moderately severe NPDR and severe NPDR, (DRSS 47 or 53, all without CI-DME at Baseline) will be enrolled.

### Investigational Plan

This is a global, Phase 3 multicenter double-masked, parallel-group, sham-controlled (needleless injection without fluid) clinical study in DR subjects without CI-DME. Subjects will be randomized 1:1 to surabgene lomparvovec or sham control. Topical steroids or artificial tears will be started 3 days prior to receiving surabgene lomparvovec or sham at Day 1 and subsequently tapered over the following 7 weeks post Day 1.

The study consists of 2 parts. Part 1 is the randomized, masked, controlled portion, and Part 2 is the open-label extension portion that is intended to provide follow-up for up to 5 years for subjects who received surabgene lomparvovec. In general, only subjects who received surabgene lomparvovec are strongly encouraged to continue the trial into Part 2; control arm subjects may exit the study after completing Week 144 or Early Discontinuation. An Independent Data Monitoring Committee will be used to monitor subject safety and trial integrity.

Randomization will be stratified by region (Japan vs. NA vs. RoW) and by baseline factors:
DRSS levels of 47 or 53 (as assessed by the central reading center [CRC])
NVE on UWF-FA (as assessed by the CRC]

### Key Eligibility Criteria

### Inclusion Criteria

### 1. Systemic

a. Individuals ≥18 years of age (including individuals of child-bearing potential).
b. Diabetes mellitus Type 1 or 2 with a HbA1c <12%.

### 2. Ocular (Study Eye)

a. Moderately-severe to severe NPDR (DRSS 47 or 53) for which PRP or anti-VEGF can be safely deferred for at least 6 months after SV1.
b. No evidence of high-risk characteristics associated with vision loss, per investigator.
c. Best corrected visual acuity (BCVA) ≥69 ETDRS letters (≥20/40 Snellen).

### Exclusion Criteria

1. Systemic
   a. Initiation of intensive insulin treatment (pump or multiple daily injections) within the past 6 months or plans to do so within 48 weeks of Day 1.
2. Ocular (study eye)
   a. Presence of CI-DME will be determined by SD-OCT evaluated by CRC.
      i) CRT ≥320 μm will be measured by Heidelberg Spectralis SD-OCT (conversion to equivalent measurement is required if imaging is done with another SD-OCT instrument).
   b. Neovascularization from a cause other than DR, per investigator (note: peripheral retinal neovascularization due to DR is not exclusionary and must be qualified by the CRC).
   c. Evidence or documented history of panretinal photocoagulation (PRP), focal grid laser or laser therapy for DR in the study eye.
   d. History of intravitreal therapy, including anti-VEGF therapy, within the past 6 months and documentation of more than 10 prior anti-VEGF or short acting steroid intravitreal injections within the past 36 months.

### Study Treatment/Device and Duration of Intervention:

1. A single dose of 5.0×10¹¹ GC/eye of surabgene lomparvovec will be administered using the Clearside SCS Microinjector® investigational device on Day 1.
2. A single needleless sham injection without fluid will be administered using the sham Clearside SCS Microinjector® on Day 1.
3. The duration of Part 1 of the study will be approximately 3 years and the duration of Part 2 was approximately 2 years with an overall study duration of approximately 5 years for surabgene lomparvovec subjects. For subjects who do not enter Part 2, the duration of the study will be approximately 3 years.

### Selection of Doses in the Study

Surabgene lomparvovec will be administered to subjects randomized to the active arm as a single SCS injection at a dose of 5.0×10¹¹ GC/eye and a volume of 100 L/injection, with a Clearside SCS Microinjector® investigational device at the study site under anesthesia (topical preferred).

Dose selection of surabgene lomparvovec at 5.0×10¹¹ GC/eye was based on data of 3 doses (2.5×10¹¹ GC/eye, 5.0×10¹¹ GC/eye, and 1.0×10¹² GC/eye) evaluated previously. In Study M23-414, surabgene lomparvovec at 5.0×10¹¹ GC/eye and 1.0×10¹² GC/eye were shown to have similar efficacy and significantly reduced rates of ≥2-step worsening at Week 48 compared to observational control or surabgene lomparvovec at 2.5×10¹¹ GC/eye. However, surabgene lomparvovec at 1.0×10¹² GC/eye had increased incidence of episcleritis following receipt of surabgene lomparvovec despite the use of ocular steroids (7-week taper regimen beginning the evening after surabgene lomparvovec administration). Therefore, given the similar efficacy between 5.0×10¹¹ GC/eye and 1.0×10¹² GC/eye and the lower incidence of episcleritis in eyes treated with 5.0×10¹¹ GC/eye, the surabgene lomparvovec dose chosen to be evaluated in Study M23-415 is 5.0×10¹¹ GC/eye.

### Steroid and Artificial Tear Regimens

Subjects randomized to surabgene lomparvovec or sham will receive prophylactic topical steroid drops or artificial tears, respectively, beginning 3 days prior to Day 1 (i.e., Day −3) and tapered over 7 weeks post Day 1. A competency check will be performed at Screening Visit 1 to ensure subject (or family member or caregiver) is able to instill eye drops after review of proper technique.

Difluprednate (0.05% difluprednate ophthalmic emulsion) will be used in sites in the US and Puerto Rico, and Pred Forte (1% prednisolone acetate ophthalmic suspension) will be used in countries where difluprednate is not available. In countries where difluprednate or Pred Forte is not available, dexamethasone (including dexamethasone sodium phosphate) 0.1% ophthalmic steroid will be used.

Regardless of country, all subjects randomized to sham will use the same formulation of artificial tears (AbbVie formulation 9963X). The dosing regimen of the artificial tears will be the same regimen as the topical steroid used in the specific country.

In countries where difluprednate is selected as the prophylactic topical steroid, the regimen for the topical steroid drops and artificial tears is as below:
Pre-procedure: Beginning in the morning 3 days prior to SCS administration (i.e., 3 days prior to Day 1) subjects will instill 1 drop 4 times daily in the study eye for 3 days. Note: In the event that Day 1 is rescheduled to a later date, subjects should continue this regimen until Day 1 occurs to ensure a minimum of 3 days on drops prior to SCS administration.
Post-procedure: Subjects will instill 1 drop in the study eye in the evening of SCS administration (Day 1). Then, starting Day 2:
   1 drop 4 times daily for 4 weeks, followed by
   1 drop 3 times daily for 1 week, followed by
   1 drop 2 times daily for 1 week, followed by
   1 drop 1 time daily for 1 week.

In countries where Pred Forte or dexamethasone is selected as the prophylactic topical steroid, the regimen for the topical steroid drops and artificial tears is as below:
Pre-procedure: Beginning in the morning 3 days prior to SCS administration (i.e., 3 days prior to Day 1) subjects will instill 2 drops 4 times daily in the study eye for 3 days. Note: In the event that Day 1 is rescheduled to a later date, subjects will continue this regimen until Day 1 occurs.
Post-procedure: subjects will instill 2 drops in the study eye in the evening of SCS administration (Day 1). Then, starting Day 2:
   2 drops 4 times daily for 4 weeks, followed by
   2 drops 3 times daily for 1 week, followed by
   2 drops 2 times daily for 1 week, followed by
   2 drops 1 time daily for 1 week.

Note: subjects who instill 2 drops are instructed to wait approximately 5 minutes after instilling the 1^{st} drop to instill the 2^{nd} drop.

### 7.3.2 Study Drug and Study Devices

TABLE 9
Description of Study Drugs.

| | | Dosage | Route of | | |
|---|---|---|---|---|---|
| Study Drug | Strength | Form | Administration | Manufacturer | Masking |
| surabgene | 5.0 × 10¹² GC/ml* | Suspension | Suprachoroidal | IDT | Open label |
| lomparvovec | | for Injection | | Biologika** | |

*Dose level: 0.1 ml (=5.0 × 10¹¹ GC/eye) one time dose
**IDT Biologika is in Rockville, MD, USA

TABLE 10
Description of Auxiliary Medicinal Products.

| | | | Route of | | |
|---|---|---|---|---|---|
| Description | Strength | Dosage Form | Administration | Manufacturer | Masking |
| DUREZOL ® | 0.05% | Eye drop | Topical into | Akorn Labs** | Masked |
| (difluprednate | | emulsion, | Conjunctival | on behalf of | |
| ophthalmic emulsion) | | multidose | Sac of Eye | Novartis | |
| Dexamethasone | 0.1% | Eye drop | Topical into | Various | Masked |
| (including | | solution, | Conjunctival | | |
| dexamethasone | | multidose^{a} | Sac of Eye | | |
| sodium phosphate), | | | | | |
| ophthalmic solution | | | | | |
| PRED FORTE ® | 1% | Eye drop | Topical into | Abb Vie | Masked |
| (prednisolone | | suspension, | Conjunctival | | |
| acetate), ophthalmic | | multidose | Sac of Eye | | |
| suspension | | | | | |
| Abb Vie formulation | N/A | Eye drop | Topical into | Abb Vie | Masked |
| 9963X^{b} | | solution, | Conjunctival | | |
| | | multidose | Sac of Eye | | |

^{a}Can also be preservative-free depending on manufacturer (dosage form: Eye drop solution, multi-dose, preservative-free)
^{b}Brand names vary and include e.g. Refresh Digital, Optive Advanced and Optive Plus.
**Akorn Labs is in Lake Forest, IL, USA

### All Medical Devices Provided by Sponsor

### Investigational Devices

AbbVie will provide Clearside SCS Microinjector for the administration of surabgene lomparvovec and Clearside SCS Microinjector Sham to simulate injection for the control arm.

TABLE 11
Description of Study Devices

| | | | Model | |
|---|---|---|---|---|
| Description | Usage | Manufacturer | Number | Masking |
| Clearside SCS | Suprachoroidal | Clearside** | RGX0911 | Open label |
| Microinjector ® | Injection | | | |
| | of surabgene | | | |
| | lomparvovec | | | |
| Clearside SCS | Simulated | Clearside** | CLSDSHAM | Open label |
| Microinjector ® | Suprachoroidal | | | |
| Sham | Injection | | | |

**Clearside Biomedical, Alpharetta, GA, USA; Other medical devices provided by sponsor (as needed)

### Packaging and Labeling

Surabgene lomparvovec will be supplied in vials in cartons. Clearside devices (Microinjector and Microinjector Sham) will be supplied in trays in cartons. Eye drops will be supplied in bottles in cartons.

Each vial, bottle, tray, and carton will be labeled as required per country requirements.

The labels must remain affixed to the vials, bottles, trays, and cartons. All blank spaces should be completed by site staff prior to dispensing to subject.

### Storage and Disposition of Study Treatments and Medical Devices

Surabgene lomparvovec must be refrigerated at 2° to 8° C./35.6° to 46.4° F.

For auxiliary medicinal products and devices, please refer to the clinical label for storage conditions.

The investigational products are for investigational use only and are to be used only within the context of this study. The study treatment and all devices supplied for this study must be maintained under adequate security and stored under the conditions specified on the label until dispensed for subject use, returned to the sponsor used or unused, or destroyed on site as instructed.

Sites are responsible for maintaining the investigational study treatment, auxiliary medicinal products and devices according to the storage conditions specified on the clinical label and monitoring for temperature excursions with the use of a calibrated continuous temperature monitoring device (for example, chart recorders and/or acceptable calibrated min/max thermometers) or continuous monitoring systems. Specific guidance on appropriate temperature monitoring and temperature excursions reporting requirements will be provided separately.

### Selection and Timing of Dose for Each Subject

For subjects randomized to surabgene lomparvovec, a single dose of 5.0×10¹¹ GC/eye of surabgene lomparvovec will be administered using the Clearside SCS Microinjector® investigational device on Day 1.

For subjects randomized to the control arm, a single, needleless sham injection without fluid will be administered using the Clearside SCS Microinjector® Sham on Day 1.

Beginning in the morning 3 days prior to SCS ABBV-RGX-314 or sham administration (i.e., 3 days prior to Day 1) subjects will initiate topical steroid drops (if randomized to ABBV-RGX-314) or artificial tears (if randomized to sham control) 4 times daily in the study eye for 3 days, gradually decreasing in a tapered regimen over a total of 7 weeks.

### 7.3.3 Discontinuation of Study Treatment and Subject Discontinuation/Withdrawal

### Withdrawal of Subjects and Discontinuation of Study

### Discontinuation of Study Intervention

Discontinuation of study intervention does not apply to subjects who receive surabgene lomparvovec as they will receive a single SCS injection on a single day.

In rare instances, it may be necessary for subjects to permanently discontinue the topical prophylactic regimen. If the topical prophylactic regimen is discontinued, the subject will remain in the study and the reason for stopping the regimen will be documented in the eCRF

If any of the following events occur, a Stopping Rule will be considered to have been met and dosing of any new subjects will be suspended until a complete review of all safety data has been performed by AbbVie and the IDMC:
The subject dies and the investigator considered the death to be related to study treatment.
Any occurrence of a TEAE of arterial thromboembolism in a subject who received study treatment that the investigator considers to be related to the study treatment.
An ocular AE that was reported as life-threatening or fatal, regardless of relationship to treatment, or reported as a severe AE that was considered related to study treatment (by the investigator).
An acute or near-term severe vision loss, defined as a ≥15-letter loss in vision within 4 weeks after study treatment administration that the investigator considers related to study treatment.

These events must be immediately reported to the AbbVie Medical Director.

### 7.3.4 Statistical Methods

### Primary and Key Secondary Endpoints

Analysis of the primary and key secondary efficacy endpoints defined above will be conducted in the mITT population.

The following events prior to the corresponding analysis time points are intercurrent events (ICEs) of interest that may potentially affect the existence or interpretation of the measurements for the primary efficacy endpoint and key secondary efficacy endpoints:
Primary efficacy endpoint (≥2-step worsening at Week 48): An intercurrent event treatment that may potentially impact DRSS assessment (e.g., ocular anti-VEGF injections, intraocular steroids, focal laser, PRP, vitrectomy, or retinal detachment repair
Key secondary efficacy endpoint (VTC by Week 48, 96, and 144): A treatment for a VTC*
Key secondary efficacy endpoint (PDR by Week 48, 96, and 144): A treatment for PDR*
Key secondary efficacy endpoint (CI-DME by Week 96 and 144): A treatment for CI-DME*

*The reason(s) treatment is given will be confirmed in the EDC by the site.

The following methods will be used to handle the ICEs defined above in all treatment arms.

In the main analysis of the primary efficacy endpoint and key secondary endpoints, the ICEs will be handled using composite variable strategy. Subjects who have an intercurrent event treatment for DRSS before Week 48 would be considered as having the event of ≥2-step worsening for Week 48. Subjects who have an ICE treatment for VTC (or PDR or CI-DME) would be considered as having an event of VTC (or PDR or CI-DME) at the time of the ICE treatment.

### Statistical Analyses for Efficacy/Effectiveness

### Summary and Analysis of the Primary Endpoint

The primary efficacy endpoint is occurrence of ≥2-step worsening in DRSS level at Week 48. Analysis of the primary endpoint will be conducted on the mITT and ITT population based on treatment as randomized. The primary efficacy analysis is to test the superiority of surabgene lomparvovec compared with sham in the proportion of subjects with occurrence of ≥2-step worsening in DRSS level at Week 48. The comparison between surabgene lomparvovec and sham will be performed using a Cochran-Mantel-Haenszel test with a 1-sided significance level of 0.025, stratified by baseline DRSS levels (47 or 53), and NVE on UWF-FA (as assessed by the CRC). Point estimates and 95% CIs for the difference in proportions between surabgene lomparvovec and sham will be provided.

Supplementary analysis and subgroup analysis for the primary efficacy endpoint will be specified in the SAP.

### Summary and Analysis of Secondary Endpoints

Summary and analysis of key secondary endpoints

The key secondary efficacy endpoints are:
Development of a VTC due to DR (i.e., CI-DME, PDR) by Week 48
Progression to PDR by Week 48
Development of a VTC due to DR (i.e., CI-DME, PDR) by Week 96
Progression to PDR by Week 96
Development of CI-DME by Week 96
Development of a VTC due to DR (i.e., CI-DME, PDR) by Week 144° Progression to PDR by Week 144° Development of CI-DME by Week 144

Analysis of the key secondary endpoint will be conducted on the mITT and ITT population based on treatment as randomized. The key secondary efficacy endpoints will be analyzed by treatment arm (ABBV-RGX-314 arm and sham arm) using Kaplan-Meier approach. Subjects who did not have the event (VTC or CI-DME or PDR) during the study will be censored at the last visit date prior to Week 48, 96, or 144 coinciding with each analysis timepoint. The difference of the rates between the 2 treatment arms and the 95% CI will be presented using the KM event rate, as well as, the associated log-rank test, stratified by baseline DRSS levels (47 or 53) and NVE on UWF-FA (as assessed by the CRC).

### Statistical Analyses for Safety

### Summary and Analysis of the Safety Endpoints

The safety analysis will be performed using the safety population as defined in Section 10.2 based on the treatment actually received, regardless of the treatment randomized. Safety will be assessed by summarizing AEs, SAEs, and AESIs. Other variables used for the safety assessments include, but are not limited to physical examinations, vital signs, and clinical laboratory testing. These variables, as well as their changes from Baseline, will be summarized descriptively.

Adverse events will be coded using the MedDRA dictionary. TEAEs are defined as any sign or symptom, disease, or other untoward medical event that begins or worsens during or after initiation of any study treatment (including prophylactic topical regimen, i.e., topical steroid drops or artificial tears). An overview of TEAEs, including treatment-emergent SAEs, treatment-emergent AESIs, treatment-related TEAEs and TEAEs by severity AEs by MedDRA preferred term and system organ class, AEs by maximum relationship to study treatment, and AEs by maximum severity will be summarized by number and percentage.

Full details of safety analyses will be provided in the SAP.

### Additional Analyses

Observed concentrations at assessed time points through the end of study following investigational product administration will be summarized descriptively by treatment arm for serum TP concentrations.

The proportion of subjects having anti-AAV8 antibodies (serum) and ABBV-RGX-314 TP antibodies (serum) will be summarized by treatment arm based on the number and percentage of subjects at assessed time points. A shift table will be presented to compare the baseline titer value to the postbaseline titer value at assessed time points.

### Interim Analysis

No formal interim analysis is planned for this study.

### Overall Type I Error Control

The statistical hypotheses comparing surabgene lomparvovec and sham for the primary efficacy endpoint and the key secondary endpoints will be tested with a fixed sequence testing procedure to ensure experiment-wise type I error rate (1-sided significance level of 0.025) is controlled according to the order specified in Table 16. Statistically significant results for the comparison in the higher rank are necessary to initiate the testing of the next comparison in the lower rank. If the statistical test for any endpoint fails to achieve statistical significance, the testing procedure will stop and no subsequent endpoint(s) following the testing sequence will be tested.

TABLE 16
Testing Sequence for Primary and Key Secondary Endpoints

| | Endpoints | Test Order |
|---|---|---|
| Primary | Occurrence of ≥2-step worsening in | 1 |
| | DRSS level at Week 48 | |
| Key | Development of a VTC due to | 2 |
| Secondary | diabetic retinopathy (i.e., CI-DME, | |
| | PDR) by Week 48 | |
| | Progression to PDR by Week 48 | 3 |
| | Development of a VTC due to | 4 |
| | diabetic retinopathy (i.e., CI-DME, | |
| | PDR) by Week 96 | |
| | Progression to PDR by Week 96 | 5 |
| | Development of CI-DME by | 6 |
| | Week 96 | |
| | Development of a VTC due to | 7 |
| | diabetic retinopathy (i.e., CI-DME, | |
| | PDR) by Week 144 | |
| | Progression to PDR by Week 144 | 8 |
| | Development of CI-DME by | 9 |
| | Week 144 | |

### 7.4 Example 4: Updated Phase 2, Randomized, Controlled, Dose-Escalation Study to Evaluate the Efficacy, Safety, and Tolerability of RGX-314 Gene Therapy Delivered Via a Single Suprachoroidal Space (SCS) Injection in Participants with Diabetic Retinopathy (DR) with and without Center Involved-Diabetic Macular Edema (CI-DME) (ALTITUDE Also Known as M23-414)

### 7.4.1 Introduction

This Example is an update to Examples 12 and 13 in PCT application publication WO2024073669, which is incorporated by reference in its entirety, and in Example 2 herein. In those Examples, Cohorts 1-5 were described. Of those cohorts, Cohorts 4 and 5 were designed to evaluate the efficacy, safety, and tolerability of SCS administration of surabgene lomparvovec (referred to a “Construct II” therein) at a dose of 1.0×10¹² GC/eye (dose level 3, or DL3) in patients without CI-DME, and where subjects were stratified by DRSS severity level with subjects having a DRSS score of 47 or 53 (moderately severe and severe NPDR, respectively) being in Cohort 4 and subjects having a DRSS score of 61 or 65 (mild and moderate PDR, respectively). In particular, the subjects in those cohorts started a 7 week tapering regimen of topical DUREZOL® (or an equivalent steroid) eye drops treatment beginning the day of SCS administration of surabgene lomparvovec (i.e., concomitantly with the surabgene lomparvovec administration, not before it). In contrast, Cohorts 1-3 did not have any specific steroid regimen.

### 7.4.2 Objectives and Endpoints

The objectives and endpoint in this updated Example are described in the table below.

| | |
|---|---|
| Objectives | Endpoints |

Primary

| | | |
|---|---|---|
| Efficacy | To evaluate the | Mean change from baseline in BCVA in the |
| | effect of surabgene | study eye at Week 54 |
| | lomparvovec on | |
| | BCVA at Week 54 | |

Secondary

| | | |
|---|---|---|
| Efficacy | To evaluate the | Mean change from baseline in BCVA in the |
| | effect of surabgene | study eye over time |
| | lomparvovec on | Proportion of participants with BCVA of 20/32 |
| | BCVA over time | or better (ETDRS ≥69 letters) in the study eye |
| | | over time |
| | To evaluate the | Proportion of participants with a 1-step or |
| | effect of surabgene | greater, a 2-step or greater, or a 3-step or |
| | lomparvovec on DR | greater worsening in DR in the study eye per |
| | (ETDRS-DRSS) | the ETDRS-DRSS on 4-widefield digital |
| | over time | stereoscopic fundus photography at Week 14, |
| | | Week 30, Week 38, and Week 54 |
| | | Proportion of participants achieving a 2-step or |
| | | greater improvement in DR in the study eye |
| | | per the ETDRS-DRSS on 4-widefield digital |
| | | stereoscopic fundus photography at Week 14, |
| | | Week 30, Week 38, and Week 54 |
| | | Proportion of participants achieving a 0-step |
| | | (no change), a 1-step or greater, a 2-step or |
| | | greater, or a 3-step or greater improvement in |
| | | DR in the study eye per the ETDRS-DRSS on |
| | | 4-widefield digital stereoscopic fundus |
| | | photography at Week 66 and Week 82 |
| | | (CO participants) |
| | | Proportion of participants with a 1-step or |
| | | greater, a 2-step or greater, or a 3-step or |
| | | greater worsening in DR in the study eye per |
| | | the ETDRS-DRSS on 4-widefield digital |
| | | stereoscopic fundus photography at Week 66 |
| | | and Week 82 (CO participants) |
| | | Mean change from baseline in the study eye in |
| | | ETDRS-DRSS severity steps at Week 22, |
| | | Week 38, and Week 54 and (CO participants) |
| | | change from Week 56 at Week 74 and |
| | | Week 82 |
| | To evaluate the | Proportion of participants with an absence of |
| | effect of surabgene | CI-DME in the study eye at Week 54 |
| | lomparvovec on | |
| | CST (as determined | |
| | by SD-OCT | |
| | measurement) at | |
| | Week 54 | |
| Safety | To assess the safety | Incidences of overall and ocular AEs |
| | and tolerability of | Vector shedding analysis in serum, urine, and |
| | surabgene | tears |
| | lomparvovec | |
| | To evaluate the | Proportion of participants who experience |
| | incidences of ocular | ocular inflammation in the study eye following |
| | inflammation | SCS surabgene lomparvovec administration |
| | following SCS | |
| | surabgene | |
| | lomparvovec | |
| | administration | |
| Safety/Efficacy | To evaluate the | Proportion of participants requiring any |
| | need for additional | additional intervention in the study eye for |
| | SOC intervention | ocular diabetic complications to Week 54 and |
| | due to ocular | (CO participants) Week 82 |
| | diabetic | Proportion of participants with any |
| | complications | sight-threatening ocular diabetic complications |
| | | in the study eye to Week 54 and (CO participants) |
| | | Week 82; for this population, the |
| | | following endpoint will be evaluated: |
| | | Duration of time to development of |
| | | sight-threatening ocular conditions |
| | | from Day 1 and (CO participants) |
| | | Week 56 |
| | | Proportion of participants developing ocular |
| | | diabetic complications in the study eye |
| | | requiring treatment per SOC (anti-VEGF |
| | | treatment, PRP, laser, or steroids) through |
| | | Week 54 and (CO participants) Week 82; for |
| | | this population, the following endpoints will be |
| | | evaluated: |
| | | Number of treatments received |
| | | Duration of time from study |
| | | intervention (Day 1 and [CO participants] |
| | | Week 56) to first treatment per SOC |
| | | Proportion of participants developing ocular |
| | | diabetic complications in the study eye |
| | | (e.g., neovascularization due to DR) requiring |
| | | treatment per SOC (PRP or anti-VEGF |
| | | treatment) through Week 54 and (CO participants) |
| | | Week 82; for this population, the |
| | | following endpoints will be evaluated: |
| | | Duration of time from study |
| | | intervention (Day 1 and [CO participants] |
| | | Week 56) to first treatment |
| | | Proportion of participants requiring |
| | | more than 1 treatment |
| | | Proportion of participants developing ocular |
| | | diabetic complications in the study eye (e.g., |
| | | retinal detachment) requiring surgical |
| | | intervention per SOC through Week 54 and |
| | | (CO participants) Week 82; for this population, |
| | | the following endpoint will be evaluated: |
| | | Duration of time from study |
| | | intervention (Day 1 and [CO participants] |
| | | Week 56) to surgical intervention |
| | To evaluate the | Mean change from baseline in CST in the |
| | effect of surabgene | study eye on SD-OCT at Week 30 and |
| | lomparvovec on | Week 54 |
| | anatomic outcomes | Mean change from Week 54 in CST in the |
| | assessed using SD- | study eye on SD-OCT at Week 82 |
| | OCT in all | (CO participants) |
| | surabgene | Proportion of participants achieving ≤290 μm |
| | lomparvovec-treated | in CST in the study eye on SD-OCT at |
| | participants | Week 30 and Week 54 |
| Pharmacodynamics | To measure aqueous | Aqueous surabgene lomparvovec TP |
| | and serum | concentration at assessed time points |
| | surabgene | Serum surabgene lomparvovec TP |
| | lomparvovec TP | concentration at assessed time points |
| | concentrations | |

Exploratory

| | | |
|---|---|---|
| Efficacy/Safety | To evaluate the | Proportion of participants with visual stability |
| | effect of surabgene | (within 5 ETDRS letters or ± 5 ETDRS letters) |
| | lomparvovec on | in the study eye from baseline to Week 54 and |
| | vision outcomes | (CO participants) from Week 56 to Week 82 |
| | (BCVA in all | Proportion of participants with vision gain or |
| | surabgene | vision loss >5 ETDRS letters in the study eye |
| | lomparvovec-treated | from baseline to Week 54 and (CO participants) |
| | participants) | from Week 56 to Week 82 |
| | To evaluate the | Proportion of participants with clinically |
| | effect of surabgene | significant changes in visual field in the study |
| | lomparvovec on | eye from baseline to Week 54, as determined |
| | visual field in all | by the investigator |
| | surabgene | |
| | lomparvovec-treated | |
| | participants | |
| Pharmacodynamics | To evaluate the | Association of baseline positive serum AAV8 |
| | effect of baseline | NAb results with aqueous humor and serum |
| | positive serum | surabgene lomparvovec TP concentrations |
| | AAV8 NAb results | |
| | on the concentration | |
| | of surabgene | |
| | lomparvovec TP in | |
| | aqueous humor and | |
| | serum | |
| Immunogenicity | To assess the | Immunogenicity measurements (AAV8: |
| | immunogenicity of | anti-AAV8 antibodies [serum] and ELISpot; |
| | surabgene | surabgene lomparvovec TP: anti-surabgene |
| | lomparvovec | lomparvovec TP antibodies [serum] and |
| | | ELISpot) |
| PRO | To evaluate visual | Mean change from Week 54 in the |
| Questionnaires | function, general | supplemented NEI VFQ-25 at Week 82 |
| | health-related QOL, | (CO participants) |
| | and patient | Mean change from Week 54 in EQ-5D-5L at |
| | impression of | Week 82 (CO participants) |
| | severity/change | Mean change from Week 54 in EQ VAS score |
| | using PRO | at Week 82 (CO participants) |
| | questionnaires | Proportion of participants with each level of |
| | | severity in overall problems with vision (PGIS |
| | | score [Week 54 and Week 82 for CO participants]) |
| | | Proportion of participants with each level of |
| | | change in problems with vision over time |
| | | (PGIC score [Week 82 for CO participants]) |

AAV8 = adeno-associated virus serotype 8;
AE = adverse event;
BCVA = best-corrected visual acuity;
CI-DME = center involved-diabetic macular edema;
CO = crossover;
CRC = central reading center;
CST = central subfield thickness;
DR = diabetic retinopathy;
DRSS = Diabetic Retinopathy Severity Scale;
ELISpot = enzyme-linked ImmunoSpot;
ETDRS = Early Treatment Diabetic Retinopathy Study;
Nab = neutralizing antibody;
NEI VFQ-25 = National Eye Institute Visual Functioning Questionnaire 25-item Version;
PGIC = Patient Global Impression of Change;
PGIS = Patient Global Impression of Severity;
PRO = patient-reported outcome;
PRP = panretinal photocoagulation;
QoL = quality of life;
SCS = suprachoroidal space;
SD-OCT = spectral domain-optical coherence tomography;
SOC = standard of care;
TP = transgene product;
VAS = Visual Analogue Scale;
VEGF = vascular endothelial growth factor

### 7.4.3 Study Design

In this phase 2, randomized (3:1), dose-escalation, observation-controlled study, approximately 100 participants with DR will be enrolled into 3 dose cohorts. Cohorts 1-5 will receive Construct II (n=15 each in Cohorts 1, 2, 4, and 5; and n=20 in Cohort 3) and approximately 20 total participants across Cohorts 1, 2, 4, and 5 will be evaluated as observational controls (n=5 per cohort). The control arm participants in Cohorts 1 and 2 will also serve as the control for Cohort 3, which will not be randomized.

All participants in Cohorts 1 and 2 will have negative or low serum titer results (<300) for AAV8 NAbs and will be randomized to either receive Construct II (at a dose of 2.5×10¹¹ GC/eye [Cohort 1] or 5.0×10¹¹ GC/eye [Cohort 2]) or be evaluated as observational controls. All participants in Cohort 3 will have a serum titer result >300 for AAV8 NAbs and will receive Construct II at a dose of 5.0×10¹¹ GC/eye (i.e., the same dose level given to participants in Cohort 2). The participants in both Cohorts 4 and 5 will receive a dose level of Construct II (1.0×10¹² GC/eye) that is higher than either of the doses tested in Cohorts 1-3. Enrollment into Cohorts 4 and 5 will be stratified by the central reading center (CRC)-confirmed Screening Visit 2 ETDRS-DRSS level. Eligible Cohort 4 participants will require an ETDRS-DRSS level 47 or 53. Cohort 5 participants require an ETDRS-DRSS level 61 or 65, with a minimum of 5 participants at each PDR level. Both Cohorts 4 and 5 will be randomized to either receive Construct II at dose level 3 (1.0×10¹² GC/eye), or be evaluated as observational controls.

Participants in the observation control arms (Cohorts 1, 2, 4, and 5) will be offered, if eligible, an opportunity to receive Construct II treatment after completion of the study. Control

Participants will be strongly encouraged to participate in a long-term follow-up (LTFU) study for continued safety evaluation. A separate informed consent for the follow-up study will be signed at that time.

For Cohorts 1-3, certain required screening test results (ie, AAV8 NAb and AAV8 total binding antibody (TAb) titers and ETDRS-DRSS score from 4-widefield fundus photography [bilateral]), formerly collected at Screening Visit 1 or obtained via the participant's enrollment in a separate NAb screening study, will be obtained exclusively through the participant's enrollment in the study. With this change, Screening Visits 1 and 2 in this study may be combined into a single screening visit and considered as Screening Visit 2. For a participant to be eligible for screening in the study, they must have an AAV8 NAb titer from the study and an ETDRS-DRSS score from the study within reasonable range of the inclusion criteria specified below. Fundus photography will be repeated in the study at Screening Visit 2 to ensure a CRC-determined, qualifying ETDRS-DRSS score is obtained. AAV8 NAbs will be measured at baseline (pretreatment) in Cohorts 4 and 5, but will not be used for screening purposes to determine eligibility and enrollment. Further, Cohorts 4 and 5 participants will not be required to enroll in a separate NAb screening study. Screening ETDRS-DRSS scores to determine study eligibility for Cohorts 4 and 5 will be obtained at Screening Visit 2. Participants who do not meet the entry criteria will be considered screen failures.

Participants receiving Construct II will have their study intervention administered at the study center during the Day 1 visit. Construct II will be given as a single injection by SCS delivery using the Clearside SCS Microinjector investigational device. The Treatment Period of the study begins at the time of Construct II administration. All investigators will be trained on the SCS procedure. A detailed description of the procedure can be found in the SCS Administration Manual.

All Cohort 4 and Cohort 5 participants randomized to Construct II will receive a protocol-mandated steroid regimen to be administered following SCS Construct II administration.

Participants receiving Construct II (all cohorts) will have 2 visits for postinjection safety (1 day postprocedure and 1 week postprocedure). Participants randomized to observation control or Construct II (Cohorts 1, 2, 4, and 5) will have their first postrandomization visit at Week 4, along with all participants enrolled to receive Construct II (Cohort 3). Following the Week 4 visit, all participants will have a Week 12 visit followed by visits every 12 weeks through Week 48. Starting on Day 2, all participants, regardless of treatment assignment, may receive approved anti-vascular endothelial growth factor (VEGF) supplemental therapy or other SOC therapy if they have ocular diabetic complications warranting intervention.

Enrollment will begin with the randomization of 2 sentinel participants in Cohort 1: one sentinel participant will be randomized to Construct II at dose level 1 and the other to observation control. The Construct II sentinel participant will be observed for safety during the 2-week postadministration period. Following this, the Sponsor's Internal Safety Committee (ISC) will review the 2-week postadministration safety data for the sentinel participant receiving Construct II and, if no safety review triggers (SRTs) are observed, the remaining participants within Cohort 1 will be randomized to either Construct II dose level 1 (n=14) or observation control (n=4).

Once all Construct II participants within Cohort 1 are enrolled, the Independent Data Monitoring Committee (IDMC) will review the available cumulative safety data from Cohort 1 (dose level 1) inclusive of the 2-week postadministration safety data from the last dosed Construct II Cohort 1 participant to determine if enrollment of dose level 2 may be initiated. During any safety review, the IDMC may recommend halting dosing, proceeding with the same or lower dose level, or proceeding to the next planned dose level. If the decision is made to dose escalate and upon full enrollment of Cohort 1, randomization of 2 sentinel participants in Cohort 2 will begin, again with one randomized to Construct II (this time at dose level 2) and the other to observation control. The ISC will review 2-week postadministration safety data from the sentinel participant receiving Construct II and, if there are no observed SRTs, the remaining participants within Cohort 2 will be randomized to either Construct II dose level 2 (n=14) or observation control (n=4). In addition, simultaneous enrollment of Cohort 3 (n=20) may also begin after the ISC approval from the Construct II sentinel participant, with all participants in the cohort receiving Construct II at dose level 2 (ie, the same dose level of Construct II evaluated in Cohort 2).

Once all Construct II participants within Cohort 2 are enrolled, the IDMC will review the available cumulative safety data inclusive of the 2-week postadministration safety data from the last dosed Construct II Cohort 2 participant to determine if enrollment of dose level 3 may be initiated. If the decision is made to dose escalate and upon full enrollment of Cohort 2, randomization of 2 sentinel participants in Cohort 4 or 5 will begin, again with one randomized to Construct II (this time at dose level 3), and the other to observation control. The ISC will review the 2-week postadministration safety data for the Construct II-treated sentinel participant in Cohort 4 (or Cohort 5) and, if there are no observed SRTs, the remaining participants within Cohorts 4 and 5 (n=38) may be enrolled in parallel.

The dose escalation plan is designed to ensure that all eligible participants assigned to receive Construct II having a negative or low serum titer result (<300) for screening serum AAV8 NAbs will complete dosing of Construct II at a given dose level, followed by IDMC review, before escalation may occur to the next dose level of Construct II. Prior to any dose escalation, the IDMC will review available cumulative safety data, inclusive of the 2-week postadministration safety visit from the last dosed Construct II participant within the dose level having a negative or low serum titer result for screening serum AAV8 NAbs.

For participants in the Construct II treatment arms, immunogenicity to the vector (as assessed by anti-AAV8 antibodies [serum], anti-Construct II TP antibodies [serum], and enzyme-linked ImmunoSpot [ELISpot] [whole blood]), and Construct II TP concentrations (aqueous humor and serum) will be assessed throughout the study.

Efficacy will be the primary focus over the 48-week study period (primary study period), as assessed through the ETDRS-DRSS score on 4-widefield digital stereoscopic fundus photography, visual acuity, and SD-OCT. Participants will be evaluated for safety through the assessment of adverse events (AEs), including serious adverse events (SAEs) and adverse events of special interest (AESIs), as well as assessments of clinical laboratory tests (chemistry, hematology, coagulation, urinalysis), vector shedding (tears, urine, and serum), and ocular examinations and imaging (BCVA, intraocular pressure [IOP], slit-lamp biomicroscopy, indirect ophthalmoscopy, ultra-widefield FA, ultra-widefield Optos fundus autofluorescence [FAF], ultra-widefield Optos color fundus photography [CFP], 4-widefield digital stereoscopic fundus photography, Humphrey visual field or microperimetry, and SD-OCT). Note that AEs will be collected at all study visits. Participants who show evidence of new retinal hypo/hyper pigmentation changes as compared with baseline will be monitored using SD-OCT scans.

Planned safety monitoring of the study participants will be conducted on an ongoing basis. The monitoring will include reviews conducted by the Medical Monitor and routine reviews conducted by the Sponsor's ISC. Separately, an IDMC will also be established and will meet on a periodic basis to independently review the clinical data.

### 7.4.4 Inclusion Criteria

Participants must meet all the following criteria in order to be eligible for this study. All ocular criteria refer to the study eye:
1. Men or women 25-89 years of age with DR secondary to diabetes mellitus Type 1 or
2. Participants must have a hemoglobin Ale <12% (as confirmed by laboratory assessments obtained at Screening Visit 2 or by a documented laboratory report dated within 60 days prior to Screening Visit 2).
   For Cohorts 1-3, study eye with moderately-severe NPDR, severe NPDR, or mild PDR (ETDRS-DRSS levels 47, 53, or 61 using standard 4-widefield digital stereoscopic fundus photographs, as determined by the CRC) for which PRP or anti-VEGF injections can be safely deferred, in the opinion of the investigator, for at least 6 months after Screening Visit 2.
   For Cohort 4, study eye with moderately-severe NPDR or severe NPDR (ETDRS-DRSS levels 47 or 53) or for Cohort 5, study eye with mild PDR or moderate PDR (ETDRS-DRSS levels 61 or 65) using standard 4-widefield digital stereoscopic fundus photographs, for which PRP or anti-VEGF injections can be safely deferred, in the opinion of the investigator, for at least 6 months after Screening Visit 2.
2. No evidence in the study eye of high-risk characteristics typically associated with vision loss, per the investigator, including the following:
   New vessels within 1-disc area of the optic nerve
   Vitreous or preretinal hemorrhage associated with less extensive new vessels at the optic disc, or with new vessels elsewhere that are half a disc area or more in size.
   No evidence in the study eye of anterior segment (e.g., iris or angle) neovascularization on clinical examination.
3. Participants in Cohorts 1 and 2 must have a negative or low serum titer result (<300) for AAV8 NAbs. Participants in Cohort 3 must have a serum titer result >300 for AAV8 NAbs. Participants in Cohorts 4 and 5 do not have an eligibility requirement based on screening AAV8 NAbs.
4. Best-corrected visual acuity in the study eye of >69 ETDRS letters (approximate Snellen equivalent 20/40 or better); note: if both eyes are eligible, the study eye must be the participant's worse-seeing eye, as determined by the investigator, prior to enrollment.
5. Prior history of CI-DME in the study eye is acceptable if no intravitreal anti-VEGF or short-acting steroid injections have been given within the last 6 months, AND no more than 10 documented injections have been given in the 3 years prior to Screening Visit 2.
6. Sexually active male participants with female partners of childbearing potential must be willing to use condoms plus a medically accepted form of partner contraception from Screening Visit 2 until 4 weeks after vector administration.
7. Must be willing and able to comply with all study procedures and be available for the duration of the study.
8. Must be willing and able to provide written, signed informed consent.

### 7.4.5 Exclusion Criteria

Participants are excluded from the study if any of the following criteria apply:
1. Women of childbearing potential (ie, women who are not postmenopausal or surgically sterile) are excluded from this clinical study.
   Postmenopausal is defined to be documented 12 consecutive months without menses.
   Surgically sterile is defined as having bilateral tubal ligation/bilateral salpingectomy, bilateral tubal occlusive procedure, hysterectomy, or bilateral oophorectomy.
2. Presence of any active CI-DME, as determined by the investigator, on clinical examination or within the central subfield thickness (CST) of the study eye, as determined by SD-OCT evaluated by CRC, using the following threshold:
   Heidelberg Spectralis: CST greater than 320 μm
3. Neovascularization in the study eye from a cause other than DR, per investigator.
4. Evidence in the study eye of optic nerve pallor on clinical examination, as determined by the investigator.
5. Any evidence or documented history of PRP or retinal laser in the study eye.
6. Ocular or periocular infection in the study eye that may interfere with the SCS procedure.
7. Any ocular condition in the study eye that could require surgical intervention within the 6 months after Screening Visit 2 (vitreous hemorrhage, cataract, retinal traction, epiretinal membrane, etc.) or any condition in the study eye that may, in the opinion of the investigator, increase the risk to the participant, require either medical or surgical intervention during the study to prevent or treat vision loss, or interfere with the study procedures or assessments.
8. Active or history of retinal detachment in the study eye.
9. Presence of an implant in the study eye at Screening Visit 2 (excluding intraocular lens).
10. Participants who had a prior vitrectomy surgery.
11. Cohorts 1-3: Advanced glaucoma in the study eye, as defined by an IOP >23 mmHg, not controlled by 2 lOP-lowering medications, any invasive procedure to treat glaucoma (e.g., shunt, tube, or minimally invasive glaucoma surgery devices; however, selective laser trabeculectomy and argon laser trabeculoplasty are permitted), or visual field loss encroaching on central fixation. Cohorts 4-5: Active or history of glaucoma or ocular hypertension in the study eye, defined as IOP >21 mmHg.
12. History of intraocular surgery in the study eye within 12 weeks prior to Screening Visit 2; yttrium aluminum garnet (YAG) capsulotomy is permitted if performed >10 weeks prior to Screening Visit 2.
13. History of intravitreal therapy in the study eye, including anti-VEGF therapy, within 6 months prior to Screening Visit 2, and documentation of more than 10 prior anti-VEGF or short-acting steroid intravitreal injections in the study eye within 36 months of Screening Visit 2.
14. Any prior intravitreal steroid injection in the study eye within 6 months prior to Screening Visit 2, administration in the study eye of Ozurdex® within 12 months prior to Screening Visit 2, or administration in the study eye of Iluvien® within 36 months prior to Screening Visit 2.
15. Any prior systemic anti-VEGF treatment within the 6 months prior to or plans to use systemic anti-VEGF therapy during the next 48 weeks after Screening Visit 2.
16. History of therapy known to have caused retinal toxicity, or concomitant therapy with any drug that may affect VA or with known retinal toxicity, e.g., chloroquine or hydroxychloroquine.
17. Myocardial infarction, cerebrovascular accident, or transient ischemic attacks within the 6 months prior to Screening Visit 2.
18. Uncontrolled hypertension (systolic blood pressure [BP]>180 mmHg, diastolic BP >100 mmHg) despite maximal medical treatment; note that if BP is brought below 180/100 mmHg and stabilized by antihypertensive treatment, as determined by the investigator and/or primary care physician, the participant can be rescreened for eligibility.
19. A systemic condition that, in the opinion of the investigator, would preclude participation in the study (poor glycemic control, uncontrolled hypertension, etc.).
20. Any concomitant treatment that, in the opinion of the investigator, may interfere with the ocular procedure or the healing process.
21. History of malignancy with or without therapy or hematologic malignancy that may compromise the immune system requiring chemotherapy and/or radiation in the 5 years prior to Screening Visit 2. Localized basal cell carcinoma will be permitted.
22. Has a serious, chronic, or unstable medical or psychological condition that, in the opinion of the investigator, may compromise the participant's safety or ability to complete all assessments and follow-up in the study.
23. Any laboratory values at Screening Visit 2 that, in the opinion of the investigator and Medical Monitor, may compromise the participant's safety or ability to complete all assessments and follow-up in the study. In the event Screening Visit 2 test results are unavailable prior to randomization or enrollment, the investigator, with the approval of the Medical Monitor, may consider historical laboratory test results collected any time within the 6 months prior to Screening Visit 2, except for HbA1c testing for which a historical lab result is acceptable only with a documented laboratory report dated within 60 days prior to Screening Visit 2.
24. History of chronic renal failure requiring dialysis or kidney transplant.
25. Initiation of intensive insulin treatment (pump or multiple daily injections) within the 6 months prior to Screening Visit 2 or plans to do so within 48 weeks of Day 1.
26. Participation in any other gene therapy study, including Construct II, or receipt of any investigational product within 30 days prior to enrollment or 5 half-lives of the investigational product, whichever is longer, or any plans to use an investigational product within 6 months following enrollment.
27. Known hypersensitivity to ranibizumab or any of its components.

### 7.4.6 Study Intervention(s) Administered

Eligible participants will be assigned either to receive a single dose of Construct II (Dose 1, Dose 2, or Dose 3) in the study eye or be followed for observation only. Cohort 1 comprises patients having negative or low (<300) NAb serum titers for AAV8, and receives Dose 1 (2.5×10¹¹ GC/eye) in a single 100 μL SCS injection. Cohort 2 comprises patients having negative or low (<300) NAb serum titers for AAV8, and receives Dose 2 (5.0×10¹¹ GC/eye) in a single 100 μL SCS injection. Cohort 3 comprises patients having positive or higher (>300) NAb serum titers for AAV8, and receives Dose 2 (5.0×10¹¹ GC/eye) in a single 100 μL SCS injection. Cohort 4 comprises patients having an ETDRS-DRSS level 47 or 53, and receives Dose 3 (1.0×10¹² GC/eye) in a single 100 μL SCS injection, and also receives a steroid treatment regime described herein below. Cohort 5 comprises patients having an ETDRS-DRSS level 61 or 65, and receives Dose 3 (1.0×10¹² GC/eye) in a single 100 μL SCS injection, and also receives a steroid treatment regime described herein below. Screening NAb titers is not used to determine eligibility in Cohorts 4 and 5. Information regarding Construct II follows.

TABLE 17
Information regarding Construct II

| Arm | Construct II | Construct II | Construct | |
|---|---|---|---|---|
| Name | Dose 1 | Dose 2 | Dose 3 | DUREZOL ® |
| Type | Gene therapy (AAV8.CB7.CL.amd42.RBG) | | | Drug (topical |
| | | | | corticosteroid) |
| Dose | Solution | | | Ophthalmic |
| Formulation | | | | emulsion |
| Unit Dose | Cohort 1: | Cohorts 2 & 3: | Cohorts 4&5: | 0.5 mg/mL |
| Strength | 2.5 × 10¹² GC/mL | 3.0 × 10¹³ GC/mL | 3.0 × 10¹³ GC/mL | (0.05%) |
| | | (diluted to 5.0 × 10¹² | (dilutedt o 1.0 × 10¹³ | |
| | | GC/mL) | GC/mL) | |
| Dose | 2.5 × 10¹¹ GC/eye | 5.0 × 10¹¹ GC/eye | 1.0 x 10¹² | 1 drop |
| Level(s) | delivered via a | delivered via a | GC/eye delivered | (see below for |
| | single 100 μL SCS | single 100 μL SCS | via a single 100 μL | dosing regimen) |
| | injection (100 μL | injection (100 μL | SCS injection | |
| | total volume) | total volume) | (100 μL total | |
| | | | volume) | |
| Route of | Suprachoroidal space injection(s) in the study eye using the | | | Topical |
| Administration | Clearside SCS Microinjector ™ investigational device | | | administration of |
| | | | | 1 drop into the |
| | | | | conjunctival sac of |
| | | | | the study eye |
| Physical | Construct Il investigational product is supplied as a frozen, | | | DUREZOL |
| Description | sterile, single-use solution of the AAV vector active ingredient | | | (difluprednate |
| | (AAV8.CB7.CL.amd42.RBG) in a formulation buffer. The | | | ophthalmic |
| | solution appears clear to opalescent, colorless, and free of | | | emulsion) 0.05% is |
| | visible particulates at room temperature. | | | supplied as a |
| | | | | sterile, preserved, |
| | | | | aqueous topical |
| | | | | ophthalmic |
| | | | | emulsion |
| Manufacturer | Advanced BioScience Laboratories, Inc | | | Alcon ®, a Novartis |
| | | | | Company |
| Packaging | Construct II will be supplied as a | | | DUREZOL |
| and | 2-mL Crystal Zenith ® sterile, single-use solution in | | | (difluprednate |
| Labeling | stoppers and aluminum vials sealed with latex-free rubber | | | ophthalmic |
| | as required per country flip-off seals. Each vial will be labeled | | | emulsion) 0.05% is |
| | regulatory requirements. | | | a sterile, aqueous |
| | | | | topical ophthalmic |
| | | | | emulsion supplied |
| | | | | in an opaque plastic |
| | | | | bottle with a |
| | | | | controlled drop tip |
| | | | | and a pink cap; 5 |
| | | | | mL in an 8 mL |
| | | | | bottle |

### 7.4.7 Steroid Regimens

Cohort 4 and Cohort 5 participants randomized to Construct II treatment will receive a protocol-mandated postprocedure steroid regimen, as follows.

Beginning the evening of SCS administration of Construct II (Day 1), participants will instill 1 drop of difluprednate ophthalmic emulsion 0.05% (DUREZOL) in the study eye, then instill drops daily in the study eye starting on Day 2, gradually decreasing in a tapered regimen over a total of 7 weeks:
4 times daily for 4 weeks, followed by
3 times daily for 1 week, followed by
2 times daily for 1 week, followed by
1 time daily for 1 week.

For any ocular inflammatory events occurring during or after the prophylaxis period, the Sponsor Clinical Development Lead should be contacted for management/treatment recommendations.

### 7.4.8 Vector Shedding

Sampling of blood (serum), urine, and tears will be performed for Construct II participants for measurement of vector concentrations. Refer to the Investigator Laboratory Manual for additional information regarding the processing, handling, and shipping of the samples.

Shedding data collected in these biological fluids provide a shedding profile of Construct II in the target patient population and is used to estimate the potential of transmission to untreated individuals. Shedding will be measured using quantitative polymerase chain reaction.

### 7.4.9 Results

In the above ongoing study of individuals with diabetic retinopathy following either control or surabgene lomparvovec SCS injection, a summary of preliminary analysis of results for subjects in Cohorts 1 to 4 has been obtained. The subjects had moderately-severe to severe NPDR (DRSS 47 or 53). Six subjects were administered Dose Level 1 (2.5×10¹¹ GC/eye), 24 subjects were administered Dose Level 2 (5.0×10¹¹ GC/eye), and 15 subjects were administered Dose Level 3 (1×10¹² GC/eye). One patient in the Dose Level 3 cohort was randomized and dosed, but was later found to have baseline confounding disease that made them ineligible for assessment. The subjects administered Dose Level 3 received a prophylactic short-course topical steroids; the subjects administered Dose Level 1 or Dose Level 2 did not received prophylactic steroids. All of these subjects received topical steroid drops starting at Day 1 (the day of surabgene lomparvovec administration) which were tapered over 7 weeks postinjection.

Table 18 summarizes the baseline characteristics of those subjects. The baseline ocular variables refer to the study eye only.

TABLE 18
Baseline Characteristics

| | Observational | Dose | Dose | Dose | |
|---|---|---|---|---|---|
| | Control | Level 1 | Level 2 | Level 3 | Total |
| | (N = 12) | (N = 6) | (N = 24) | (N = 14) | (N = 56) |
| Mean Age (Years) | 54.9 | 57.2 | 60.1 | 54.4 | 57.2 |
| Gender-Female | 3 | 4 | 10 | 4 | 21 |
| | (25.0)% | (66.7%) | (41.7%) | (28.6%) | (37.5%) |
| Hemoglobin | 7.7 | 7.9 | 7.8 | 7.7 | 7.7 |
| A1c-Mean | | | | | |
| DR Category at | | | | | |
| Baseline | | | | | |
| DRSS 47 | 12 | 4 | 21 | 10 | 47 |
| (Moderately | (100.0%) | (66.7%) | (87.5%) | (71.4%) | (83.9%) |
| Severe NPDR) | | | | | |
| DRSS 53 | 0 | 2 | 3 | 4 | 9 |
| (Severe NPDR) | | (33.3%) | (12.5%) | (28.6%) | (16.1%) |
| Screening BCVA | 85.3 | 74.7 | 83.0 | 80.1 | 81.9 |
| (ETDRS letters) | | | | | |
| Screening OCT | 273.7 | 250.5 | 272.2 | 279.4 | 272.0 |
| CRT (μm) | | | | | |
| Lens Status- | 10 | 5 | 15 | 13 | 43 |
| Phakic n (%) | (83.3%) | (83.3%) | (62.5%) | (92.9%) | (76.8%) |

BCVA: best corrected visual acuity;
CRT: central retinal thickness;
DRSS: Diabetic Retinopathy Severity Scale:
ETDRS: Early Treatment Diabetic Retinopathy Study;
NPDR: non-proliferative diabetic retinopathy;
OCT: optical coherence tomography.

### Safety Results

Table 19 summarizes safety results from the subjects in Table 6. Through 2 years of follow-up, surabgene lomparvovec was well-tolerated by the subjects receiving Dose Levels 1, 2 and 3 (n=45). No drug-related serious adverse events were reported; of 16 serious adverse events, none was considered drug-related. No cases of pigmentary changes or cells, vasculitis, occlusion, hypotony, or chorioretinitis were reported. Surprisingly, no intraocular inflammation was reported in subjects with short course of prophylactic topical steroids, even though they received a relatively high dose of surabgene lomparvovec.

TABLE 19
Cumulative Safety Summary

| | | | Prophylactic | |
|---|---|---|---|---|
| | | | short-course | |
| | No prophylactic steroids | | topical steroids | Surabgene |
| DOSE LEVELS 1-3: | Dose Level 1 | Dose Level 2 | Dose Level 3 | Lomparvovec |
| Common Ocular TEAEs^{a} | 2.5 × 10¹¹ | 5 × 10¹¹ | 1 × 10¹² | Total |
| in the Study Eye | DRSS 47-53 | DRSS 47-53 | DRSS 47-53 | DRSS 47-53 |
| through 2 Years | (N = 6) | (N = 24) | (N = 15) | (N = 45) |
| Conjunctival Hyperemia | 1 (16.7%) | 7 (29.2%) | 0 (0%) | 8 (17.8%) |
| Conjunctival Hemorrhage | 1 (16.7%) | 3 (12.5%) | 3 (20.0%) | 7 (15.6%) |
| Cataract | 2 (33.3%) | 3 (12.5%) | 1 (6.7%) | 6 (13.3%) |
| Episcleritis^{b} | 0 (0.0%) | 3 (12.5%) | 3 (20.0%) | 6 (13.3%) |
| IOP increase^{c} | 0 (0.0%) | 2 (8.3%) | 3 (20.0%) | 5 (11.1%) |
| Intraocular Inflammation^{d} | 1 (16.7%) | 2 (8.3%) | 0 (0%) | 3 (6.7%) |

^{a}Common TEAEs include AEs occurring in ≥10% patients + intraocular inflammation with onset through the 2-year visit; includes 2 patients who missed the 2-year visit but had data at 2.5 years.
^{b}All cases were mild to moderate (grade 1-2) and have resolved on topical corticosteroids based on slit lamp examination.
^{c}All cases were mild to moderate (grade 1-2) and resolved after steroid discontinuation and are off IOP lowering medications, except one case of bilateral Primary Angle Closure Glaucoma (grade 2) unrelated to drug which was treated with bilateral iridectomy.
^{d}All cases were mild (range +0.5 to +1) and most presented 2-6 weeks post injection, predominantly as anterior cells on slit lamp examination. Resolved on topical corticosteroids.
AE: adverse event;
DRSS: Diabetic Retinopathy Severity Scale;
IOI. intraocular inflammation;
IOP: intraocular pressure;
NPDR: non-proliferative diabetic retinopathy;
SAE: serious adverse event;
TEAE: treatment emergent adverse event.

The absence of any intraocular inflammation or pigmentation in subjects who were administered Dose Level 3 with a short-course prophylactic topical steroids was unexpected. More particularly, the 15 subjects were each assessed with respect to (1) anterior chamber cells, flare, or pigmented cells, and (2) vitreous cells, flare, or pigmented cells, at the following time points after administration of surabgene lomparvovec: day 2; week 1; month 1; month 3; month 6; month 9; year 1; year 1.5; and year 2. The assessment could be scored as No; Trace; 1+; 2+; 3+; 4+. For each of the 15 subjects, the assessments indicated No intraocular inflammation or pigmentation in either the anterior segment or the vitreous at each of the time points.

TABLE 20
Anterior Chamber Cells, Flare, Or Pigmented Cells

| Subject | | | | | | | | | |
|---|---|---|---|---|---|---|---|---|---|
| 1 | No | No | No | No | No | No | No | No | No |
| 2 | No | No | No | No | No | No | No | No | No |
| 3 | No | No | No | No | No | No | No | No | No |
| 4 | No | No | No | No | No | No | No | No | No |
| 5 | No | No | No | No | No | No | No | No | No |
| 6 | No | No | No | No | No | No | No | No | No |
| 7 | No | No | No | Nc | No | No | No | No | No |
| 8 | No | No | No | No | No | No | No | No | No |
| 9 | No | No | No | No | No | No | No | No | No |
| 10 | No | No | No | No | No | No | No | No | No |
| 11 | No | No | No | No | No | No | No | No | No |
| 12 | No | No | No | No | No | No | No | No | No |
| 13 | No | No | No | No | No | No | No | No | No |
| 14 | No | No | Nc | No | No | No | No | No | No |
| 15 | Nc | No | No | No | No | No | No | No | No |
| | D2 | W1 | M1 | M3 | M6 | M9 | Y1 | Y1.5 | Y2 |

TABLE 21
Vitreous Cells, Flare, Or Pigmented Cells

| Subject | | | | | | | | | |
|---|---|---|---|---|---|---|---|---|---|
| 1 | No | No | No | No | No | No | No | No | No |
| 2 | No | No | No | No | No | No | No | No | No |
| 3 | No | No | No | No | No | No | No | No | No |
| 4 | No | No | No | No | No | No | No | No | No |
| 5 | No | No | No | No | No | No | No | No | No |
| 6 | No | No | No | No | No | No | No | No | No |
| 7 | No | No | No | No | No | No | No | No | No |
| 8 | No | No | No | No | No | No | No | No | No |
| 9 | No | No | No | No | No | No | No | No | No |
| 10 | No | No | No | No | No | No | No | No | No |
| 11 | No | No | No | No | No | No | No | No | No |
| 12 | No | No | No | No | No | No | No | No | No |
| 13 | No | No | No | No | No | No | No | No | No |
| 14 | No | No | No | No | No | No | No | No | No |
| 15 | No | No | No | No | No | No | No | No | No |
| | D2 | W1 | M1 | M3 | M6 | M9 | Y1 | Y1.5 | Y2 |

As illustrated above, administration of surabgene lomparvovec to subjects in need of treatment of a retinal disease associated with hVEGF such as DR resulted in safety results in the form of prevention of ocular inflammation. Such safety results were at each of the following time points after administration of surabgene lomparvovec: day 2; week 1; month 1; month 3; month 6; month 9; year 1; year 1.5; and year 2. The single dose of surabgene lomparvovec was safe and efficacious at year 2 after administration.

### Efficacy Results

At one (1) year after administration of surabgene lomparvovec or control, the diabetic retinopathy severity scale (DRSS) scores of subjects were graded in the course of long term follow-up in the study. Table 22 shows the numbers of subjects having worsening DRSS scores, no change, or improved DRSS scores in the groups receiving the aflibercept control or surabgene lomparvovec SCS injection at Dose Level 1, Dose Level 2, or Dose Level 3.

TABLE 22
Change in DRSS at 1 Year

| | ≥3 Steps | 2 Steps | 1 Step | | 1 Step | 2 Steps | ≥3 Steps |
|---|---|---|---|---|---|---|---|
| | ←←←←←←←DR Worsening | | | No Change | DR Improving→→→→→→→ | | |
| Control (n = 12) | 3* | 2 | 1 | 4 | 1 | 1 | 0 |
| Dose Level 1 (n = 6) | 0 | 1 | 0 | 1 | 2 | 2 | 0 |
| Dose Level 2 (n = 24) | 0 | 0 | 0 | 7* | 12 | 5 | 0 |
| Dose Level 3 (n = 14)** | 0 | 0 | 2* | 3 | 6* | 2 | 1 |

*Includes one subject who received on-study anti-VEGF.
**One subject in Dose Level 3 was found to have confounding disease at baseline and their data was excluded.
One patient each in Dose Levels 1 and 2 missed their 1-Year visit, so their 6-month results were used.

In the group of subjects receiving a control treatment, 41.7% of those subjects had a ≥2-step worsening in DRSS score, and only 8.3% had a ≥2-step improvement in DRSS score. In the group of subjects receiving surabgene lomparvovec SCS injection at Dose Level 1, 16.7% of those subjects had a ≥2-step worsening in DRSS score, and 33.3% had a ≥2-step improvement in DRSS score. In the group of subjects receiving surabgene lomparvovec SCS injection at Dose Level 2, 0% of those subjects had a ≥2-step worsening in DRSS score, and 20.8% had a ≥2-step improvement in DRSS score. In the group of subjects receiving surabgene lomparvovec SCS injection at Dose Level 3, 0% of those subjects had a ≥2-step worsening in DRSS score, and 21.4% had a ≥2-step improvement in DRSS score. The groups of subjects receiving Dose Levels 2 and 3 performed similarly, with none of the subjects having ≥2-step worsening in DRSS score.

As illustrated in Table 22, administration of surabgene lomparvovec to subjects in need of treatment of a retinal disease associated with hVEGF such as DR resulted in efficacy results at one year from administration in the form of subjects showing an improvement in their DRSS score relative to baseline, and/or subjects not showing a worsening in their DRSS score of two steps or more relative to baseline, and/or subjects having either a one-step reduction in their DRSS score, no change in their DRSS score, or a one-step improvement in their DRSS score.

At two (2) years after administration of surabgene lomparvovec (or one year after administration of the control treatment), the diabetic retinopathy severity scale (DRSS) scores of subjects were graded in the course of long term follow-up in the study. Table 23 shows the percentages of subjects having worsening DRSS scores, no change, or improved DRSS scores in the groups receiving the aflibercept control or surabgene lomparvovec SCS injection at Dose Level 2 or Dose Level 3.

TABLE 23
Change in DRSS at 2 Years

| | ≥2 Steps DRSS | ≥2 Steps DRSS |
|---|---|---|
| | Worsening | Improving |
| Control (n = *12) | *42% | *8% |
| Dose Level 2 (n = 17) | 12% | 35% |
| Dose Level 3 (n = 11) | 0% | 73% |

*Observed Data at 1 year

Four subjects from Dose Level 2 group and three subjects from Dose Level 3 group received intravitreal (IVT) anti-VEGF treatment by year 2. Three subjects did not enroll in long-term follow-up.

At two years after administration of surabgene lomparvovec, subjects treated with Dose Level 3 outperformed all other groups. Surprisingly, none of the subjects treated with Dose Level 3 had ≥2 Steps DRSS worsening, thus administration of surabgene lomparvovec at that dose had an efficacy result of subjects not showing a worsening in their DRSS scores of two steps or more relative to baseline. Almost three-quarters (73%) of subjects at Dose Level 3 had ≥2 Steps DRSS improvement, thus administration of surabgene lomparvovec at that dose level also had efficacy results at two years from administration in the form of subjects showing an improvement in their DRSS scores relative to baseline. Subjects treated with Dose Level 2 also had efficacy results at two years from administration in the form of subjects not showing a worsening in their DRSS scores of two steps or more relative to baseline and/or subjects showing an improvement in their DRSS scores relative to baseline.

Subjects were also assessed for reduction of supplemental anti-VEGF injection at two years after administration of surabgene lomparvovec (or one year after administration of the control treatment) in the course of long term follow-up in the study. Table 24 shows percentages of subjects having greater than or equal to 2 step improvement in DRSS without intravitreal ranibizumab supplemental therapy at 2 year after administration of surabgene lomparvovec.

TABLE 24
≥2-Step DRSS Improvement Without Supplemental IVT at 2 Years

| Year 1 | | Year 2 | |
|---|---|---|---|
| Study Control^{a} (n = 12) (1 Y) | 8% | Historical Control^{b} | 8% |
| Dose Level 2 (n = 23) | 21.7% | Dose Level 2 (n = 17) | 18% |
| Dose Level 3 (n = 14) | 21.4% | Dose Level 3 (n = 11) | 55% |

^{a}Control subjects crossed over the receive surabgene lomparvovec at Year 1
^{b}Protocol W Control rate (n = 164)

At two years after administration of surabgene lomparvovec, subjects treated with Dose Level 3 outperformed all other groups in reduction of supplemental anti-VEGF injection. Surprisingly, the percentage of subjects without supplemental anti-VEGF injection at two years was seven-fold (7×) higher in the group receiving surabgene lomparvovec at Dose Level 3 compared to the historical control group. The percentage of subjects without supplemental anti-VEGF injection at 1 and 2 years after receiving surabgene lomparvovec at Dose Level 2 was also much higher than the control groups. Thus, administration of surabgene lomparvovec at Dose Level 2 and Dose Level 3 had efficacy results in the form of subjects not developing ocular diabetic complications requiring treatment per standard of care (SOC) for such complications in the eye treated with surabgene lomparvovec, and/or subjects showing a reduction in a supplemental anti-VEGF injection annualized rate after the administration of the surabgene lomparvovec, and/or subjects receiving no more than 2, no more than 1, or no supplemental anti-VEGF injections after the administration of the surabgene lomparvovec.

Additionally a majority of subjects without supplemental anti-VEGF injection had ≥2 step improvements over the long term. Table 25 shows DRSS scores in subjects receiving Dose Level 3 at one and two years after administration of surabgene lomparvovec.

TABLE 25
DRSS Outcomes in Dose Level 3 Subjects over 2 Years

| | Baseline | | | |
|---|---|---|---|---|
| | DRSS | Year 1 | Year 2 | |
| Subjects | 53 | 2 steps better | 3 steps better | ≥2 Steps |
| with no | 47 | 2 steps better | 2 steps better | DRSS |
| supplemental | 47 | 1 step better | 2 steps better | improvement |
| anti-VEGF | 47 | 1 step better | 2 steps better | at Year 2 |
| injection | 53 | No change | 2 steps better | |
| | 47 | 1 step better | 2 steps better | |
| | 47 | 1 step better | 1 step better | |
| | 47 | 1 step better | 1 step better | |
| | 53 | 3 steps better* | | |
| | 47 | 1 step better* | | |
| | 47 | 1 step worse* | | |
| With suppl | 47 | 1 step better | 4 steps better | |
| anti-VEGF | 47 | No change | 2 steps better | |
| injection | 53 | 1 step worse | 1 step worse | |

*Subject did not enroll in long-term follow-up

Subjects were also assessed for vision threatening events at two years after administration of surabgene lomparvovec in the course of long term follow-up in the study. Table 26 shows percentages of subjects for whom vision threatening events were reported.

TABLE 26
Vision Threatening Events at 2 Years

| | | | | |
|---|---|---|---|---|
| Historical Controls | PANORAMA (n = 133) Protocol W (n = 164) surabgene lomparvovec DL1 (n = 6) surabgene lomparvovec DL2 (n = 24) surabgene lomparvovec DL3 (n = 14) | 50.4% 47.8% 33.0% 25.0% 14.3% | [symbol] | >70% Reduction |

Data shown is using LOCF.
VTEs = VTCs + CI-DME; VTCs could include PDR or ASNV.
Data shown is using LOCF.
Historical controls include VTC + CI-DME.
ASNV: anterior segment neovascularization;
CI-DME: center-involved diabetic macular edema;
DL: dose level;
LOCF: Last Observation Carried Forward;
PDR: proliferative diabetic retinopathy;
VTCs: vision-threatening complications;
VTEs: vision-threatening events.

To briefly summarize preliminary analysis of results obtained for subjects with NPDR in Cohorts 1 to 4 of the ALTITUDE study at two years after administration of surabgene lomparvovec, single in-office suprachoroidal injection of surabgene lomparvovec was well-tolerated at Dose Levels 1, 2 and 3. Surprisingly, no intraocular inflammation (IOI) was reported in Dose Level 3 with short-course topical steroids.

Durable, long-term efficacy was observed, as a majority of subjects receiving surabgene lomparvovec achieved DRSS improvement, with 55% of subjects receiving surabgene lomparvovec at Dose Level 3 achieving ≥2-step improvement without supplemental anti-VEGF injections, and risk of disease progression was reduced, with ≥70% risk reduction in vision threatening events in subjects receiving surabgene lomparvovec at Dose Level 3.

Moreover, the preliminary analysis of results in the clinical study indicates that in certain embodiments, administering a single dose of surabgene lomparvovec to the suprachoroidal space of an eye of the subject in an amount up to 1.0×10¹² GC/eye has efficacy results that are the subject having either no change in their DRSS score, or a one-step improvement in their DRSS score, or a two-step improvement in their DRSS score, or a three-step improvement in their DRSS score, or a four-step improvement in their DRSS score. In certain embodiments, administering a single dose of surabgene lomparvovec to the suprachoroidal space of an eye of the subject in an amount up to 1.0×10¹² GC/eye has efficacy results that are the subject having a ≥2 step improvement in their DRSS score at year 1 or at year 2 after administration of the surabgene lomparvovec when compared to their DRSS score when the surabgene lomparvovec was administered. In certain embodiments, administering a single dose of surabgene lomparvovec to the suprachoroidal space of an eye of the subject in an amount up to 1.0×10¹² GC/eye has efficacy results that are the subject receiving no more than 2, no more than 1, or no supplemental anti-VEGF injections in year 1, or in years 1 and 2, after the administration of the surabgene lomparvovec. In certain embodiments, administering a single dose of surabgene lomparvovec to the suprachoroidal space of an eye of the subject in an amount up to 1.0×10¹² GC/eye has efficacy results that are the subject receives no supplemental anti-VEGF injections in year 1, or in years 1 and 2, after the administration of the surabgene lomparvovec, and the subject has a ≥2 step improvement in their DRSS score.

It is believed that at a single dose of surabgene lomparvovec greater than 1.0×10¹² GC/eye, administering a topical steroid (e.g., difluprednate or prednisolone acetate) at least 3 days and up to 7 days prior to administration of a single dose of surabgene lomparvovec to the eye of the subject results in an unexpected amount of mitigation of inflammation associated with the surabgene lomparvovec when compared to the administration of surabgene lomparvovec at such a dose without administering a topical steroid at least 3 days and up to 7 days prior to administration of a single dose of surabgene lomparvovec. Whereas administration of a topical steroid concomitantly as described in this example is shown to be unexpectedly beneficial when administering a single dose of surabgene lomparvovec in an amount up to 1.0×10¹² GC/eye, prophylactic administration of a topical steroid at least 3 days and up to 7 days prior to administration of a single dose of surabgene lomparvovec in an amount equal to or greater than 1.0×10¹² GC/eye may be further beneficial. As described herein, suprachoroidal administration of surabgene lomparvovec in amounts equal to or greater than 1.0×10¹² GC/eye may result in increasing occurrences of ocular inflammation, intraocular inflammation, and/or episcleritis in the subject. Thus, administration of therapeutically effective amounts of a topical steroid to the eye of the subject at least 3 days and up to 7 days prior to administration of the single dose of surabgene lomparvovec ameliorates and/or prevents such ocular inflammation, intraocular inflammation, and/or episcleritis, especially where the single dose of surabgene lomparvovec is greater than 1.0×10¹² GC/eye.

### 7.5 Example 5: Multicenter, Randomized, Masked, Sham-Controlled Study to Evaluate Efficacy and Safety of Surabgene Lomparvovec Delivered Via Suprachoroidal Space (SCS) Injection Targeting Subjects with Diabetic Retinopathy without Center Involved-Diabetic Macular Edema (CI-DME) (NAAVIGATE Also Known as M23-415)

### Objectives and Endpoints

The primary objective and endpoint are as follows:

The primary objective is to evaluate the efficacy of a single suprachoroidal space administration of surabgene lomparvovec compared with sham in subjects with DRSS level 47 or 53 without CI-DME at baseline.

The primary efficacy endpoint of the Phase 2b portion is the achievement of ≥2-step improvement in DRSS level in the study eye at Week 52.

The safety objectives are to assess the safety and tolerability of surabgene lomparvovec and to evaluate the incidence of ocular inflammation following suprachoroidal space surabgene lomparvovec administration.

The safety endpoints are as follows:

Occurrence of ocular and non-ocular adverse events (AEs)/serious adverse events (SAEs)/adverse events of special interest (AESIs).

Occurrence of intraocular inflammation in the study eye.

Occurrence of scleral inflammation including episcleritis in the study eye.

The key secondary objective is to evaluate the development of vision threatening events (VTEs) in subjects who received surabgene lomparvovec compared with sham.

The key secondary efficacy endpoints are as follows:

Development of a VTE due to DR (i.e., CI-DME, progressive diabetic retinopathy [PDR], anterior segment neovascularization [ASNV]) in the study eye by Week 52.

Progression to PDR or ASNV in the study eye by Week 52.

Development of CI-DME in the study eye by Week 52.

Development of a VTE due to DR (i.e., CI-DME, PDR, ASNV) in the study eye by week 104.

Achievement of ≥2-step improvement in DRSS level in the study eye at Week 104.

Progression to PDR or ASNV in the study eye by Week 104.

Development of CI-DME in the study eye by Week 104.

### Investigational Plan

The study aims to evaluate the efficacy and safety of treatment with surabgene lomparvovec administered using the Clearside SCS Microinjector investigational device. The studies include a Masked Period, beginning at the time of randomization to surabgene lomparvovec or sham through 2 years post dose.

The studies will include a long-term follow-up (LTFU) extension (LTFU Period) that is intended to provide safety and efficacy follow-up for up to 5 years post dose for the subjects who receive surabgene lomparvovec.

One part of the study is randomized, masked, multicenter, parallel-group, sham-controlled clinical study to evaluate the efficacy and safety of surabgene lomparvovec. To evaluate the efficacy and safety of 2 regimens of steroid eye drops for prophylaxis of ocular inflammation, subjects will also be randomly assigned to 1 or 2 steroid regimens: difluprednate 0.05% ophthalmic emulsion or prednisolone acetate 1% ophthalmic suspension.

Subjects will be randomized in a 2:1 ratio to either surabgene lomparvovec DL3 (1.0×10¹² GC/eye) or sham needleless control. Within each group, subjects will be allocated 1:1 to either steroid regimen, for a total of 4 treatment arms. The randomization ratio for the 4 treatment arms will be 2:2:1:1.

Another part of the study is a randomized, masked, multicenter, parallel-group, sham-controlled clinical study in subjects with DR without CI-DME who have DRSS level 47 or 53 to evaluate the efficacy and safety of surabgene lomparvovec. Subjects will be randomized in a 1:1 ratio to either surabgene lomparvovec DL3 (1.0×10¹² GC/eye) or sham needleless control. administration.

### Study Treatment/Device and Duration of Intervention:

For subjects randomized to active treatment, surabgene lomparvovec will be administered in the suprachoroidal space of the study eye on Day 1 at a dose of 1×10¹² GC/eye (DL3) using the Clearside SCS Microinjector investigational device. For subjects randomized to control, a single needleless sham injection without fluid will be administered to the study eye on Day 1 using the Clearside Sham SCS Microinjector. The study duration will be approximately 61 months for subjects who receive surabgene lomparvovec, or longer (depending on the duration of follow-up after receiving sham injection) for subjects in the sham control groups who cross over to receive treatment with surabgene lomparvovec.

### Selection of Doses in the Study

Surabgene lomparvovec will be administered to subjects randomized to the active arm as a single suprachoroidal space injection at 1×10¹² GC/eye in a volume of 100 μL/injection, with a Clearside SCS Microinjector investigational device at the study site under local anesthesia (topical preferred).

Selection of the surabgene lomparvovec dose level (DL) 1.0×10¹² GC/eye (DL3) is based on data from Studies M23-414 and M23-420. Study M23-414 evaluated 3 doses of surabgene lomparvovec (DL1=2.5×10¹¹ GC/eye, DL2=5.0×10¹¹ GC/eye, and DL3=1.0×10¹² GC/eye) in subjects with DR without CI-DME. Study M23-420 is the long-term safety and efficacy study evaluating surabgene lomparvovec-treated subjects from Study M23-414.

In Study M23-414, surabgene lomparvovec at doses of 2.5×10¹¹ GC/eye, 5.0×10¹¹ GC/eye, and 1.0×10¹² GC/eye achieved similar rates of ≥2-step DRSS improvement at Week 48. Notably, the 1.0×10¹² GC/eye DL demonstrated greater long-term efficacy; in the M23-420 LTFU study, the proportion of subjects achieving ≥2-step DRSS improvement from baseline at DL3 increased to 45.5% (5/11) at Year 2, while maintaining low rates of VTEs.

Based on interim data from the ongoing Study M23-414, a 20% incidence rate of episcleritis was reported at 1×10¹² GC/eye and no intraocular inflammation was observed in the NPDR population (n=15) following a short course of prophylactic topical corticosteroids (7-week taper beginning the evening after administration). In the total population of subjects with DR without CI-DME receiving 1×10¹² GC/eye (n=29), the incidences of episcleritis and intraocular inflammation based on interim data, including the LTFU period (M23-420) were 24.1% and 6.9%, respectively.

### Steroid and Artificial Tear Regimens

For prophylaxis of ocular inflammation, subjects in part of the study will be given topical steroid drops if randomized to surabgene lomparvovec, or, to maintain masking, artificial tears if randomized to sham. On Day −7, the subjects will be instructed via phone call to begin a daily prophylactic regimen with taper of topical steroid drops or artificial tears up through 12 weeks post dose.

For this dosing, the branded drug product is preferred for use if available (i.e., Durezol® and Pred Forte®). To maintain masking, subjects randomized to the sham control arms will receive artificial tear eye drops (Formulation 9963X, also known as Refresh® Digital) to mimic steroid use in the active treatment arms.

Difluprednate 0.05% Ophthalmic Emulsion (Phase 2b, Phase 3, and Bilateral Portions).

Pre-procedure: Beginning in the morning 7 days prior to suprachoroidal space surabgene lomparvovec administration (i.e., 7 days prior to Day 1), study subjects will instill 1 drop of difluprednate ophthalmic emulsion 0.05% (DUREZOL) 4 times daily in the study eye for 1 week.

Note: In the event that Day 1 is rescheduled to a later date, subjects should continue this regimen until Day 1 occurs to ensure a minimum of 3 days on drops prior to suprachoroidal space administration.

Day-of/Post-procedure: Starting the day of surabgene lomparvovec administration, participants will instill 1 drop in the study eye in the evening of suprachoroidal space administration (Day 1) Then, starting Day 2, initiate a 12-week taper regimen where they will instill drops in their study eye daily, gradually decreasing in a tapered regimen over a total of 12 weeks as follows:
1 drop 4 times daily for 4 weeks, followed by
1 drop 3 times daily for 2 weeks, followed by
1 drop 2 times daily for 2 weeks, followed by
1 drop 1 time daily for 2 weeks, followed by
1 drop every other day for 2 weeks.

Prednisolone Acetate 1% Ophthalmic Suspension (Phase 2b Portion).

Pre-procedure: Beginning in the morning 7 days prior to suprachoroidal space surabgene lomparvovec administration (i.e., 7 days prior to Day 1), study subjects will instill 2 drops of prednisolone acetate 1% ophthalmic suspension (PRED FORTE) 6 times daily in the study eye for 1 week.

Note: In the event that Day 1 is rescheduled to a later date, subjects should continue this regimen until Day 1 occurs. The total duration of the per-protocol steroid prophylaxis regimen is 12 weeks+7 days (91 days).

Day-of/Post-procedure: Starting the day of surabgene lomparvovec administration, participants will instill 2 drops in the study eye in the evening of suprachoroidal space administration (Day 1). Then, starting Day 2, initiate a 12-week taper regimen where they will instill drops in their study eye daily, gradually decreasing in a tapered regimen over a total of 12 weeks as follows:
2 drops 6 times daily for 4 weeks, followed by
2 drops 5 times daily for 1 week, followed by
2 drops 4 times daily for 1 week, followed by
2 drops 3 times daily for 1 week, followed by
2 drops 2 times daily for 1 week, followed by
2 drops 1 time daily for 2 weeks, followed by
2 drops 1 time every other day for 2 weeks.

Note: The total duration of the per-protocol steroid prophylaxis regimen is 12 weeks+7 days (91 days). Subjects who are instilling 2 drops will be instructed to wait approximately 5 minutes after instilling the first drop to instill the second drop.

### 6.6 Example 6

An animal study in rabbits was conducted to ascertain the
pharmacokinetics of difluprednate (Durezol) and prednisolone
acetate (Pred Forte). The regimens administered are described
in the following Table.

| | | | | C_{trough} |
|---|---|---|---|---|
| | | Dose | ICB | Fold-over |
| Drug | Regimen^{a} | Amount | T_{1/2} | Kᵢ |
| Durezol | 4× daily | 17.5 μg | 2.8 hrs | 361× |
| 0.05% | (1 gtt, Q6H) | | | |
| (emulsion) | | | | |
| Pred | 6× daily | 700 μg | 2.8 hrs | 500× |
| Forte 1% | (2 gtts, Q4H) | | | |
| (suspension) | 8× daily | 350 μg | 1.9 hrs | 576× |
| | (1 gtts, Q3H) | | | |

^{a}1 gtt = 35 μL

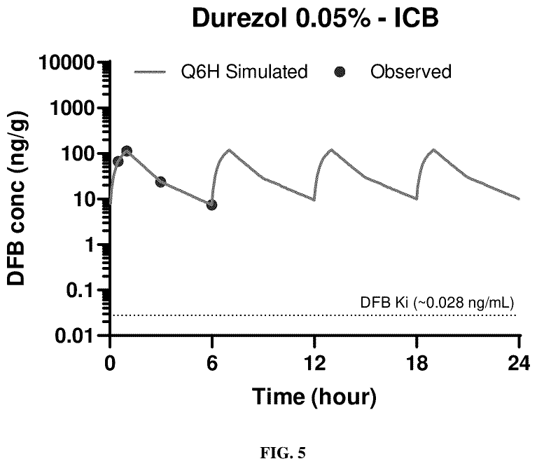

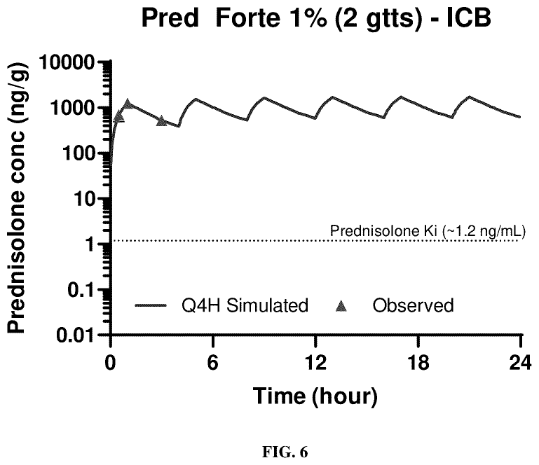

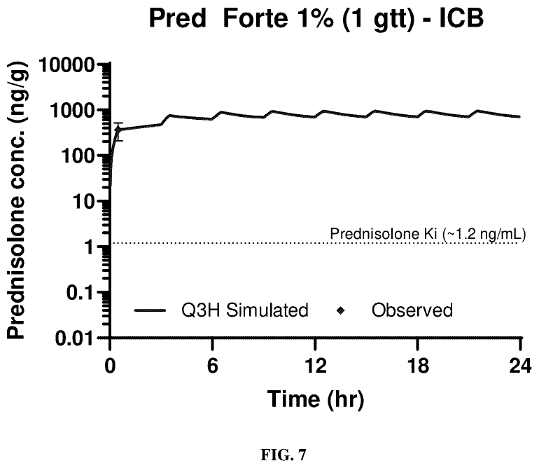

The pharmacokinetics observed are provided in FIGS. 5-7. As shown in FIGS. 5-7, administration of Pred Forte unexpectedly provided higher concentrations than Durezol.

### 8. EQUIVALENTS

Although embodiments are described in detail with reference to specific embodiments thereof, it will be understood that variations which are functionally equivalent are within the scope of this disclosure. Indeed, various modifications of embodiments described herein in addition to those shown and described herein will become apparent to those skilled in the art from the foregoing description and accompanying drawings. Such modifications are intended to fall within the scope of the appended claims. Those skilled in the art will recognize or be able to ascertain using no more than routine experimentation, many equivalents to the specific embodiments described herein. Such equivalents are intended to be encompassed by the following claims.

All publications, patents and patent applications mentioned in this specification are herein incorporated by reference into the specification to the same extent as if each individual publication, patent or patent application was specifically and individually indicated to be incorporated herein by reference in their entireties.

### 9. ILLUSTRATIVE EMBODIMENTS

Some illustrative embodiments are described below.

Illustrative Embodiment 1. A method of treating neovascular age-related macular degeneration (nAMD) in a subject in need thereof, wherein the method comprises administering surabgene lomparvovec treatment and a steroid treatment; wherein:
a. the surabgene lomparvovec treatment comprises administering a therapeutically effective amount of surabgene lomparvovec to the suprachoroidal space of an eye of the subject; and
b. the steroid treatment comprises administering a therapeutically effective amount of a steroid to the eye of the subject about three days prior to administration of the surabgene lomparvovec; and
wherein the steroid treatment enables administration of surabgene lomparvovec at doses equal to or greater than about 1×10¹² GC/eye;
wherein the method results in an efficacy result, a safety result, or both;
wherein the efficacy result is selected from the group consisting of (1) the subject having a reduction in choroidal neovascularization (CNV) lesion size and/or leakage area relative to baseline, (2) the subject having a gain of visual acuity relative to baseline, (3) the subject not having a significant loss of visual acuity relative to baseline, (4) the subject having a reduction in central retinal thickness (CRT) relative to a baseline, (5) the subject showing a reduction in a supplemental anti-VEGF injection annualized rate after the administration of the surabgene lomparvovec, and (6) the subject receiving no more than 2, no more than 1, or no supplemental anti-VEGF injections after the administration of the surabgene lomparvovec; and
wherein the safety result is mitigation of ocular inflammation of episcleritis, anterior chamber cell, vitritis, iridocyclitis, iritis, eye inflammation, anterior chamber flare, vitreal cells, keratic precipitates, and uveitis.

Illustrative Embodiment 2. The method of Illustrative Embodiment 1, wherein the steroid is administered topically.

Illustrative Embodiment 3. The method of Illustrative Embodiment 2, wherein the steroid administration continues for about 7 to 12 weeks.

Illustrative Embodiment 4. The method of Illustrative Embodiment 3, wherein the steroid is difluprednate.

Illustrative Embodiment 5. The method of Illustrative Embodiment 4, wherein the difluprednate is a 0.05% (w/v) ophthalmic emulsion of difluprednate.

Illustrative Embodiment 6. The method of Illustrative Embodiment 5 wherein the difluprednate is administered using 1 drop 4 times daily in the eye for the 3 days prior to the administration of the surabgene lomparvovec, then 1 drop in the eye the day of surabgene lomparvovec administration, and then, beginning the day after the administration of the surabgene lomparvovec, 1 drop 4 times daily for 4 weeks, followed by 1 drop 3 times daily for 1 week, followed by 1 drop 2 times daily for 1 week, and followed by 1 drop 1 time daily for 1 week.

Illustrative Embodiment 7. The method of Illustrative Embodiment 5, wherein the difluprednate is administered using 1 drop 4 times daily in the eye for the 3 days prior to the administration of the surabgene lomparvovec, and then, beginning the days of the administration of the surabgene lomparvovec, 1 drop 4 times daily for 4 weeks, followed by 1 drop 3 times daily for 2 weeks, followed by 1 drop 2 times daily for 2 weeks, followed by 1 drop 1 time daily for 2 weeks, and followed by 1 drop 1 time every other day for 2 weeks.

Illustrative Embodiment 8. The method of Illustrative Embodiment 5, wherein the difluprednate is administered using 1 drop 4 times daily in the eye for the 3 days prior to the administration of the surabgene lomparvovec, and then, beginning the days of the administration of the surabgene lomparvovec, 1 drop 4 times daily for 4 weeks, followed by 1 drop 3 times daily for 1 week, followed by 1 drop 2 times daily for 1 week, and followed by 1 drop 1 time daily for 1 week.

Illustrative Embodiment 9. The method of Illustrative Embodiment 3, wherein the steroid is prednisolone acetate or dexamethasone.

Illustrative Embodiment 10. The method of Illustrative Embodiment 9, wherein the prednisolone acetate is a 1% (w/v) prednisolone acetate ophthalmic suspension of prednisolone acetate and wherein the dexamethasone is a 0.1% (w/v) dexamethasone ophthalmic suspension, a 0.1% (w/v) dexamethasone phosphate ophthalmic suspension, or a 0.1% (w/v) dexamethasone sodium phosphate ophthalmic suspension.

Illustrative Embodiment 11. The method of Illustrative Embodiment 10, wherein the prednisolone acetate or dexamethasone is administered using 2 drops 4 times daily in the eye for the 3 days prior to the administration of the surabgene lomparvovec, then 2 drops in the eye the day of surabgene lomparvovec administration, and then, beginning the day after the administration of the surabgene lomparvovec, 2 drops 4 times daily for 4 weeks, followed by 2 drops 3 times daily for 1 week, followed by 2 drops 2 times daily for 1 week, and followed by 2 drops 1 time daily for 1 week.

Illustrative Embodiment 12. The method of Illustrative Embodiment 10, wherein the prednisolone acetate or dexamethasone is administered using 2 drops 4 times daily in the eye for the 3 days prior to the administration of the surabgene lomparvovec, and then, beginning the day of the administration of the surabgene lomparvovec, 2 drops 4 times daily for 4 weeks, followed by 2 drops 3 times daily for 2 weeks, followed by 2 drops 2 times daily for 2 weeks, followed by 2 drops 1 time daily for 2 weeks, and followed by 2 drops 1 time every other day for 2 weeks.

Illustrative Embodiment 13. The method of Illustrative Embodiment 10, wherein the prednisolone acetate or dexamethasone is administered using 2 drops 4 times daily in the eye for the 3 days prior to the administration of the surabgene lomparvovec, and then, beginning the day of the administration of the surabgene lomparvovec, 2 drops 4 times daily for 4 weeks, followed by 2 drops 3 times daily for 1 week, followed by 2 drops 2 times daily for 1 week, and followed by 2 drops 1 time daily for 1 week.

Illustrative Embodiment 14. The method of any one of the preceding Illustrative Embodiments, wherein the subject has received one or more injections of ranibizumab prior to the administration of the surabgene lomparvovec, and optionally, receives one post-treatment injection of ranibizumab after the administration of the surabgene lomparvovec.

Illustrative Embodiment 15. The method of Illustrative Embodiment 14, wherein one of the ranibizumab injections is a screening ranibizumab injection administered to the subject about 6 weeks before the administration of the surabgene lomparvovec.

Illustrative Embodiment 16. The method of Illustrative Embodiment 15, wherein about one week after the administration of the screening ranibizumab injection, the subject shows no more than 50 μm of remaining fluid height in the area within 3 mm of the parafovea as determined by SD-OCT when compared to the fluid level when the screening ranibizumab injection was administered if the subject at least 50 μm of fluid when the screening ranibizumab injection was administered, or the subject shows any amount of fluid reduction when compared to the fluid level when the screening ranibizumab injection was administered if the subject at less than 50 μm of fluid when the screening ranibizumab injection was administered.

Illustrative Embodiment 17. The method of Illustrative Embodiment 16, wherein a second ranibizumab injection is administered to the subject about four weeks after the screening ranibizumab injection was administered.

Illustrative Embodiment 18. The method of Illustrative Embodiment 17, wherein the surabgene lomparvovec is administered to the subject about 2 weeks after the second ranibizumab injection was administered, and wherein the optional post-treatment ranibizumab injection is administered about 2 weeks after the surabgene lomparvovec is administered.

Illustrative Embodiment 19. The method of any one of Illustrative Embodiments 1-18, wherein the method results in an efficacy result that is the subject having a reduction in choroidal neovascularization (CNV) lesion size and/or leakage area relative to baseline.

Illustrative Embodiment 20. The method of Illustrative Embodiment 19, wherein the reduction in choroidal neovascularization (CNV) lesion size and/or leakage area relative to a baseline CNV lesion size and/or leakage area is within or at 26, 40, or 52 weeks after the administration of the surabgene lomparvovec, and wherein the baseline CNV lesion size and/or leakage area is measured at the time of the administration of the surabgene lomparvovec or up to two weeks before.

Illustrative Embodiment 21. The method of Illustrative Embodiment 19 or 20, wherein the method results in a safety result that is mitigation of ocular inflammation of episcleritis, anterior chamber cell, vitritis, iridocyclitis, iritis, eye inflammation, anterior chamber flare, vitreal cells, keratic precipitates, and uveitis.

Illustrative Embodiment 22. The method of any one of Illustrative Embodiments 1-18, wherein the method results in an efficacy result that is the subject having a gain of visual acuity relative to baseline.

Illustrative Embodiment 23. The method of Illustrative Embodiment 22, wherein the gain of visual acuity is a gain of ≥15, ≥10, ≥5, or ≥0 letters of BCVA based on the ETDRS scale relative to a baseline number of letters within or at 52, 40, or 26 weeks after the administration of the surabgene lomparvovec, and wherein the baseline number of letters is measured at the time of the administration of the surabgene lomparvovec or up to two weeks before.

Illustrative Embodiment 24. The method of Illustrative Embodiment 22 or 23, wherein the method results in a safety result that is mitigation of ocular inflammation of episcleritis, anterior chamber cell, vitritis, iridocyclitis, iritis, eye inflammation, anterior chamber flare, vitreal cells, keratic precipitates, and uveitis.

Illustrative Embodiment 25. The method of any one of Illustrative Embodiments 1-18, wherein the method results in an efficacy result that is the subject not having a significant loss of visual acuity relative to baseline.

Illustrative Embodiment 26. The method of Illustrative Embodiment 25, wherein the subject does not have a loss of ≥15, ≥10, ≥5, or ≥0 letters of BCVA based on the ETDRS scale relative to a baseline number of letters within or at 52, 40, or 26 weeks after the administration of the surabgene lomparvovec.

Illustrative Embodiment 27. The method of Illustrative Embodiment 25 or 26, wherein the method results in a safety result that is mitigation of ocular inflammation of episcleritis, anterior chamber cell, vitritis, iridocyclitis, iritis, eye inflammation, anterior chamber flare, vitreal cells, keratic precipitates, and uveitis.

Illustrative Embodiment 28. The method of any one of Illustrative Embodiments 1-18, wherein the method results in an efficacy result that is the subject having a reduction in central retinal thickness (CRT) relative to a baseline.

Illustrative Embodiment 29. The method of Illustrative Embodiment 28, wherein the subject has a reduction in central retinal thickness (CRT) relative to a baseline CRT within or at 52, 40, or 26 weeks after the administration of the surabgene lomparvovec, and wherein the baseline CRT is measured at the time of the administration of the surabgene lomparvovec or up to two weeks before.

Illustrative Embodiment 30. The method of Illustrative Embodiment 29, wherein the reduction in CRT is no fluid relative to baseline as measured by SD-OCT.

Illustrative Embodiment 31. The method of Illustrative Embodiment 29, wherein the reduction in CRT is a fluid level that is within 30 μm of baseline.

Illustrative Embodiment 32. The method of any one of Illustrative Embodiments 28-31, wherein the method results in a safety result that is mitigation of ocular inflammation of episcleritis, anterior chamber cell, vitritis, iridocyclitis, iritis, eye inflammation, anterior chamber flare, vitreal cells, keratic precipitates, and uveitis.

Illustrative Embodiment 33. The method of any one of Illustrative Embodiments 1-13, wherein the method results in an efficacy result that is the subject showing a reduction in a supplemental anti-VEGF injection annualized rate after the administration of the surabgene lomparvovec.

Illustrative Embodiment 34. The method of Illustrative Embodiment 33, wherein the subject shows a reduction of at least 50% or 75% in a supplemental anti-VEGF injection annualized rate through week 40 or 52 after the surabgene lomparvovec administration when compared with the number of anti-VEGF injections during the 52 weeks preceding the surabgene lomparvovec administration, and wherein the subject received at least one anti-VEGF injection during the 52 weeks preceding the surabgene lomparvovec administration.

Illustrative Embodiment 35. The method of Illustrative Embodiment 33 or 34, wherein the method results in a safety result that is mitigation of ocular inflammation of episcleritis, anterior chamber cell, vitritis, iridocyclitis, iritis, eye inflammation, anterior chamber flare, vitreal cells, keratic precipitates, and uveitis.

Illustrative Embodiment 36. The method of any one of Illustrative Embodiments 1-13, wherein the method results in an efficacy result that is the subject receiving no more than 2, no more than 1, or no supplemental anti-VEGF injections after the administration of the surabgene lomparvovec.

Illustrative Embodiment 37. The method of Illustrative Embodiment 36, wherein the subject receives no more than 2, no more than 1, or no supplemental anti-VEGF injections through week 26, 40, or 52 after the surabgene lomparvovec administration.

Illustrative Embodiment 38. The method of Illustrative Embodiment 36 or 37, wherein the method results in a safety result that is mitigation of ocular inflammation of episcleritis, anterior chamber cell, vitritis, iridocyclitis, iritis, eye inflammation, anterior chamber flare, vitreal cells, keratic precipitates, and uveitis.

Illustrative Embodiment 39. The method of any one of Illustrative Embodiments 14-18, wherein the method results in an efficacy result that is the subject showing a reduction in a supplemental anti-VEGF injection annualized rate after the administration of the surabgene lomparvovec.

Illustrative Embodiment 40. The method of Illustrative Embodiment 39, wherein the subject shows a reduction of at least 50% or 75% in a supplemental anti-VEGF injection annualized rate through week 40 or 52 after the surabgene lomparvovec administration when compared with the number of anti-VEGF injections during the 52 weeks preceding the screening ranibizumab injection administration and wherein the subject received at least one anti-VEGF injection during the 52 weeks preceding the screening ranibizumab injection administration.

Illustrative Embodiment 42. The method of Illustrative Embodiment 39 or 40, wherein the method results in a safety result that is mitigation of ocular inflammation of episcleritis, anterior chamber cell, vitritis, iridocyclitis, iritis, eye inflammation, anterior chamber flare, vitreal cells, keratic precipitates, and uveitis.

Illustrative Embodiment 43. The method of any one of Illustrative Embodiments 14-18, wherein the method results in an efficacy result that is the subject receiving no more than 2, no more than 1, or no supplemental anti-VEGF injections after the administration of the surabgene lomparvovec, not including the optional post-treatment ranibizumab injection.

Illustrative Embodiment 44. The metho of Illustrative Embodiment 43, wherein the subject receives no more than 2, no more than 1, or no supplemental anti-VEGF injections through week 26, 40, or 52 after the surabgene lomparvovec administration.

Illustrative Embodiment 45. The method of any one of the preceding Illustrative Embodiments, wherein therapeutically effective amount of surabgene lomparvovec is selected from the group consisting of 1×10¹² GC/eye, 1.5×10¹² GC/eye, and 2×10¹² GC/eye.

Illustrative Embodiment 47. The method of any one of Illustrative Embodiments 1-45, wherein the subject has phakic lenses.

Illustrative Embodiment 48. The method of any one of Illustrative Embodiments 1-45, wherein the subject has pseudophakic lenses.

Illustrative Embodiment 49-1. A method of treating diabetic retinopathy (DR) with or without center involved-diabetic macular edema (CI-DME) in a subject in need thereof, wherein the method comprises administering surabgene lomparvovec treatment and a steroid treatment; wherein:
a. the surabgene lomparvovec treatment comprises administering a therapeutically effective amount of surabgene lomparvovec to the suprachoroidal space of an eye of the subject; and
b. the steroid treatment comprises administering a therapeutically effective amount of a steroid to the eye of the subject about three days prior to administration of the surabgene lomparvovec; and
wherein the steroid treatment enables administration of surabgene lomparvovec at doses equal to or greater than about 5×10¹¹ GC/eye;
wherein the method results in an efficacy result, a safety result, or both;
wherein the efficacy result is selected from the group consisting of (1) the subject maintaining best-corrected visual acuity (BCVA) relative to baseline, (2) the subject showing an improvement in their diabetic retinopathy severity scale (DRSS) score relative to baseline, (3) the subject no longer having CI-DME that was present when the surabgene lomparvovec was administered, (4) the subject not developing any sight-threatening ocular diabetic complications in the eye treated with surabgene lomparvovec, (5) the subject not developing ocular diabetic complications requiring treatment per standard of care (SOC) for such complications in the eye treated with surabgene lomparvovec, (6) if the subject develops neovascularization due to diabetic retinopathy requiring treatment per standard of care (SOC), then the subject only requires one SOC treatment to treat the neovascularization, (7) the subject not developing ocular diabetic complications requiring surgical intervention per standard of care (SOC) for such complications in the eye treated with surabgene lomparvovec, (8) the subject has a CST of less than 320 μm or no more than 290 μm after administration of the surabgene lomparvovec, (9) the subject not showing a worsening in their diabetic retinopathy severity scale (DRSS) score of two steps or more relative to baseline, (10) the subject not developing a vision-threatening complication (VTC) due to diabetic retinopathy, (11) subject having either a one-step reduction in their DRSS score, no change in their DRSS score, or a one-step improvement in their ETDRS DRSS score, (12) the subject maintains a ETDRS BCVA of within ±5 letters at week 48 after administration of the surabgene lomparvovec when compared to their ETDRS BCVA when the surabgene lomparvovec was administered, (13) the subject gains more than 5 letters of ETDRS BCVA at week 48 after administration of the surabgene lomparvovec when compared to their ETDRS BCVA when the surabgene lomparvovec was administered;
wherein the safety result is mitigation of ocular inflammation of episcleritis, anterior chamber cell, vitritis, iridocyclitis, iritis, eye inflammation, anterior chamber flare, vitreal cells, keratic precipitates, and uveitis.

Illustrative Embodiment 49-2. A method of treating diabetic retinopathy (DR) with or without center involved-diabetic macular edema (CI-DME) in a subject in need thereof, wherein the method comprises administering surabgene lomparvovec treatment and a steroid treatment; wherein:
a. the surabgene lomparvovec treatment comprises administering a therapeutically effective amount of surabgene lomparvovec to the suprachoroidal space of an eye of the subject; and
b. the steroid treatment comprises administering a therapeutically effective amount of a steroid to the eye of the subject about three days prior to administration of the surabgene lomparvovec; and
wherein the steroid treatment enables administration of surabgene lomparvovec at doses equal to or greater than about 1×10¹² GC/eye;
wherein the method results in an efficacy result, a safety result, or both;
wherein the efficacy result is selected from the group consisting of (1) the subject maintaining best-corrected visual acuity (BCVA) relative to baseline, (2) the subject showing an improvement in their diabetic retinopathy severity scale (DRSS) score relative to baseline, (3) the subject no longer having CI-DME that was present when the surabgene lomparvovec was administered, (4) the subject not developing any sight-threatening ocular diabetic complications in the eye treated with surabgene lomparvovec, (5) the subject not developing ocular diabetic complications requiring treatment per standard of care (SOC) for such complications in the eye treated with surabgene lomparvovec, (6) if the subject develops neovascularization due to diabetic retinopathy requiring treatment per standard of care (SOC), then the subject only requires one SOC treatment to treat the neovascularization, (7) the subject not developing ocular diabetic complications requiring surgical intervention per standard of care (SOC) for such complications in the eye treated with surabgene lomparvovec, (8) the subject has a CST of less than 320 μm or no more than 290 μm after administration of the surabgene lomparvovec, (9) the subject not showing a worsening in their diabetic retinopathy severity scale (DRSS) score of two steps or more relative to baseline, (10) the subject not developing a vision-threatening complication (VTC) due to diabetic retinopathy, (11) subject having either a one-step reduction in their DRSS score, no change in their DRSS score, or a one-step improvement in their ETDRS DRSS score, (12) the subject maintains a ETDRS BCVA of within ±5 letters at week 48 after administration of the surabgene lomparvovec when compared to their ETDRS BCVA when the surabgene lomparvovec was administered, (13) the subject gains more than 5 letters of ETDRS BCVA at week 48 after administration of the surabgene lomparvovec when compared to their ETDRS BCVA when the surabgene lomparvovec was administered;
wherein the safety result is mitigation of ocular inflammation of episcleritis, anterior chamber cell, vitritis, iridocyclitis, iritis, eye inflammation, anterior chamber flare, vitreal cells, keratic precipitates, and uveitis.

Illustrative Embodiment 50. The method of Illustrative Embodiment 49-1 or 49-2, wherein the steroid is administered topically.

Illustrative Embodiment 51. The method of Illustrative Embodiment 50, wherein the steroid administration continues for about 7 to 12 weeks.

Illustrative Embodiment 52. The method of Illustrative Embodiment 51, wherein the steroid is difluprednate.

Illustrative Embodiment 53. The method of Illustrative Embodiment 52, wherein the difluprednate is a 0.05% (w/v) ophthalmic emulsion of difluprednate.

Illustrative Embodiment 54. The method of Illustrative Embodiment 53 wherein the difluprednate is administered using 1 drop 4 times daily in the eye for the 3 days prior to the administration of the surabgene lomparvovec, then 1 drop in the eye the day of surabgene lomparvovec administration, and then, beginning the day after the administration of the surabgene lomparvovec, 1 drop 4 times daily for 4 weeks, followed by 1 drop 3 times daily for 1 week, followed by 1 drop 2 times daily for 1 week, and followed by 1 drop 1 time daily for 1 week.

Illustrative Embodiment 55. The method of Illustrative Embodiment 53, wherein the difluprednate is administered using 1 drop 4 times daily in the eye for the 3 days prior to the administration of the surabgene lomparvovec, and then, beginning the days of the administration of the surabgene lomparvovec, 1 drop 4 times daily for 4 weeks, followed by 1 drop 3 times daily for 2 weeks, followed by 1 drop 2 times daily for 2 weeks, followed by 1 drop 1 time daily for 2 weeks, and followed by 1 drop 1 time every other day for 2 weeks.

Illustrative Embodiment 56. The method of Illustrative Embodiment 53, wherein the difluprednate is administered using 1 drop 4 times daily in the eye for the 3 days prior to the administration of the surabgene lomparvovec, and then, beginning the days of the administration of the surabgene lomparvovec, 1 drop 4 times daily for 4 weeks, followed by 1 drop 3 times daily for 1 week, followed by 1 drop 2 times daily for 1 week, and followed by 1 drop 1 time daily for 1 week.

Illustrative Embodiment 57. The method of Illustrative Embodiment 51, wherein the steroid is prednisolone acetate or dexamethasone.

Illustrative Embodiment 58. The method of Illustrative Embodiment 57, wherein the prednisolone acetate is a 1% (w/v) prednisolone acetate ophthalmic suspension of prednisolone acetate and wherein the dexamethasone is a 0.1% (w/v) dexamethasone ophthalmic suspension, a 0.1% (w/v) dexamethasone phosphate ophthalmic suspension, or a 0.1% (w/v) dexamethasone sodium phosphate ophthalmic suspension.

Illustrative Embodiment 59. The method of Illustrative Embodiment 58, wherein the prednisolone acetate or dexamethasone is administered using 2 drops 4 times daily in the eye for the 3 days prior to the administration of the surabgene lomparvovec, then 2 drops in the eye the day of surabgene lomparvovec administration, and then, beginning the day after the administration of the surabgene lomparvovec, 2 drops 4 times daily for 4 weeks, followed by 2 drops 3 times daily for 1 week, followed by 2 drops 2 times daily for 1 week, and followed by 2 drops 1 time daily for 1 week.

Illustrative Embodiment 60. The method of Illustrative Embodiment 58, wherein the prednisolone acetate or dexamethasone is administered using 2 drops 4 times daily in the eye for the 3 days prior to the administration of the surabgene lomparvovec, and then, beginning the day of the administration of the surabgene lomparvovec, 2 drops 4 times daily for 4 weeks, followed by 2 drops 3 times daily for 2 weeks, followed by 2 drops 2 times daily for 2 weeks, followed by 2 drops 1 time daily for 2 weeks, and followed by 2 drops 1 time every other day for 2 weeks.

Illustrative Embodiment 61. The method of Illustrative Embodiment 58, wherein the prednisolone acetate or dexamethasone is administered using 2 drops 4 times daily in the eye for the 3 days prior to the administration of the surabgene lomparvovec, and then, beginning the day of the administration of the surabgene lomparvovec, 2 drops 4 times daily for 4 weeks, followed by 2 drops 3 times daily for 1 week, followed by 2 drops 2 times daily for 1 week, and followed by 2 drops 1 time daily for 1 week.

Illustrative Embodiment 62. The method of any one of Illustrative Embodiment 49-1 and Embodiments 49-2 to 61, wherein the subject has received one or more injections of ranibizumab prior to the administration of the surabgene lomparvovec.

Illustrative Embodiment 63. The method of Illustrative Embodiment 62, wherein one of the ranibizumab injections is a screening ranibizumab injection administered to the subject about 10 weeks before the administration of the surabgene lomparvovec.

Illustrative Embodiment 64. The method of Illustrative Embodiment 63, wherein about one week after the administration of the screening ranibizumab injection, the subject shows a reduction in central subfield thickness (CST) of at least 10% when compared to the CST when the screening ranibizumab injection was administered.

Illustrative Embodiment 65. The method of Illustrative Embodiment 64, wherein the subject receives one or two pre-treatment injections of aflibercept prior to the administration of the surabgene lomparvovec.

Illustrative Embodiment 66. The method of Illustrative Embodiment 65, wherein the subject receives two pre-treatment injections of aflibercept prior to the administration of the surabgene lomparvovec.

Illustrative Embodiment 67. The method of Illustrative Embodiment 66, wherein the first of the two pre-treatment aflibercept injections is administered about 4 weeks after the screening ranibizumab injection is administered and about 6 weeks before the surabgene lomparvovec is administered.

Illustrative Embodiment 68. The method of Illustrative Embodiment 67, wherein the second of the two pre-treatment aflibercept injections is administered about 4 weeks after the first pre-treatment aflibercept injection is administered and about 2 weeks before the surabgene lomparvovec is administered.

Illustrative Embodiment 69. The method of Illustrative Embodiment 68, wherein the surabgene lomparvovec is administered about 2 weeks after the second pre-treatment aflibercept injection.

Illustrative Embodiment 70. The method of Illustrative Embodiment 69, wherein the subject receives one or two post-treatment aflibercept injections after the administration of the surabgene lomparvovec.

Illustrative Embodiment 71. The method of Illustrative Embodiment 70, wherein the subject receives two post-treatment aflibercept injections after the administration of the surabgene lomparvovec.

Illustrative Embodiment 72. The method of Illustrative Embodiment 71, wherein the first of the two post-treatment aflibercept injections is administered about two weeks after the administration of the surabgene lomparvovec.

Illustrative Embodiment 73. The method of Illustrative Embodiment 72, wherein the second of the two post-treatment aflibercept injections is administered about 4 weeks after the first post-treatment aflibercept injection is administered.

Illustrative Embodiment 74. The method of any one of Illustrative Embodiment 49-1 and Embodiments 49-2 to 73, wherein the method results in an efficacy result that is the subject maintaining best-corrected visual acuity (BCVA) relative to baseline.

Illustrative Embodiment 75. The method of Illustrative Embodiment 74, wherein the subject maintains a ETDRS BCVA of 69 or more letters at week 14, 30, 38, or 54 after the surabgene lomparvovec is administered.

Illustrative Embodiment 76. The method of any one of Illustrative Embodiments 74 or 75, wherein the method results in a safety result that is mitigation of ocular inflammation of episcleritis, anterior chamber cell, vitritis, iridocyclitis, iritis, eye inflammation, anterior chamber flare, vitreal cells, keratic precipitates, and uveitis.

Illustrative Embodiment 77. The method of any one of Illustrative Embodiment 49-1 and Embodiments 49-2 to 73, wherein the method results in an efficacy result that is the subject showing an improvement in their diabetic retinopathy severity scale (DRSS) score relative to baseline.

Illustrative Embodiment 78. The method of Illustrative Embodiment 77, wherein the subject has a 1-step or greater, 2-step or greater, or 3-step or greater improvement in their DRSS score at week 12, 14, 28, 30, 38, 48, 54, 96, or 144 after administration of the surabgene lomparvovec.

Illustrative Embodiment 79. The method of Illustrative Embodiment 77 or 78, wherein the method results in a safety result that is mitigation of ocular inflammation of episcleritis, anterior chamber cell, vitritis, iridocyclitis, iritis, eye inflammation, anterior chamber flare, vitreal cells, keratic precipitates, and uveitis.

Illustrative Embodiment 80. The method of any one of Illustrative Embodiment 49-1 and Embodiments 49-2 to 73, wherein the method results in an efficacy result that is the subject no longer having CI-DME that was present when the surabgene lomparvovec was administered.

Illustrative Embodiment 81. The method of Illustrative Embodiment 80, wherein the subject has an absence of CI-DME at week 54 after administration of the surabgene lomparvovec.

Illustrative Embodiment 82. The method of Illustrative Embodiment 80 or 81, wherein the method results in a safety result that is mitigation of ocular inflammation of episcleritis, anterior chamber cell, vitritis, iridocyclitis, iritis, eye inflammation, anterior chamber flare, vitreal cells, keratic precipitates, and uveitis.

Illustrative Embodiment 83. The method of any one of Illustrative Embodiment 49-1 and Embodiments 49-2 to 73, wherein the method results in an efficacy result that is the subject not developing any sight-threatening ocular diabetic complications in the eye treated with surabgene lomparvovec.

Illustrative Embodiment 84. The method of Illustrative Embodiment 83, wherein the subject does not develop any sight-threatening ocular diabetic complications in the eye treated with surabgene lomparvovec over the period of time from the administration of the surabgene lomparvovec to week 28 or 54 after the administration of the surabgene lomparvovec.

Illustrative Embodiment 85. The method of Illustrative Embodiment 83 or 84, wherein the method results in a safety result that is mitigation of ocular inflammation of episcleritis, anterior chamber cell, vitritis, iridocyclitis, iritis, eye inflammation, anterior chamber flare, vitreal cells, keratic precipitates, and uveitis.

Illustrative Embodiment 86. The method of any one of Illustrative Embodiment 49-1 and Embodiments 49-2 to 73, wherein the method results in an efficacy result that is the subject not developing ocular diabetic complications requiring treatment per standard of care (SOC) for such complications in the eye treated with surabgene lomparvovec.

Illustrative Embodiment 87. The method of Illustrative Embodiment 85, wherein the subject does not develop ocular diabetic complications requiring treatment per standard of care (SOC) for such complications in the eye treated with surabgene lomparvovec over the period of time from the administration of the surabgene lomparvovec to week 28 or 54 after the administration of the surabgene lomparvovec, and wherein the SOC is anti VEGF treatment, PRP treatment, laser treatment, or steroid treatment.

Illustrative Embodiment 88. The method of Illustrative Embodiment 86 or 87, wherein the ocular diabetic complication is neovascularization due to diabetic retinopathy.

Illustrative Embodiment 89. The method of Illustrative Embodiment 86, 87, or 88, wherein the method results in a safety result that is mitigation of ocular inflammation of episcleritis, anterior chamber cell, vitritis, iridocyclitis, iritis, eye inflammation, anterior chamber flare, vitreal cells, keratic precipitates, and uveitis.

Illustrative Embodiment 90. The method of any one of Illustrative Embodiment 49-1 and Embodiments 49-2 to 73, wherein the method results in an efficacy result that is, if the subject develops neovascularization due to diabetic retinopathy requiring treatment per standard of care (SOC), then the subject only requires one SOC treatment to treat the neovascularization.

Illustrative Embodiment 91. The method of Illustrative Embodiment 90, wherein the subject develops neovascularization due to diabetic retinopathy requiring treatment per standard of care (SOC) for such complications in the eye treated with surabgene lomparvovec over the period of time from the administration of the surabgene lomparvovec to week 28 or 54 after the administration of the surabgene lomparvovec; wherein the SOC is anti VEGF treatment or PRP treatment, and wherein the subject only receives one SOC treatment.

Illustrative Embodiment 92. The method of Illustrative Embodiment 90 or 91, wherein the method results in a safety result that is mitigation of ocular inflammation of episcleritis, anterior chamber cell, vitritis, iridocyclitis, iritis, eye inflammation, anterior chamber flare, vitreal cells, keratic precipitates, and uveitis.

Illustrative Embodiment 93. The method of any one of Illustrative Embodiment 49-1 and Embodiments 49-2 to 73, wherein the method results in an efficacy result that is the subject not developing ocular diabetic complications requiring surgical intervention per standard of care (SOC) for such complications in the eye treated with surabgene lomparvovec.

Illustrative Embodiment 94. The method of Illustrative Embodiment 93, wherein the subject does not develop ocular diabetic complications requiring surgical intervention per standard of care (SOC) for such complications in the eye treated with surabgene lomparvovec over the period of time from the administration of the surabgene lomparvovec to week 54 after the administration of the surabgene lomparvovec.

Illustrative Embodiment 95. The method of Illustrative Embodiment 93 or 94, wherein the diabetic complications requiring surgical intervention per SOC is retinal detachment.

Illustrative Embodiment 96. The method of Illustrative Embodiment 913, 94, or 91, wherein the method results in a safety result that is mitigation of ocular inflammation of episcleritis, anterior chamber cell, vitritis, iridocyclitis, iritis, eye inflammation, anterior chamber flare, vitreal cells, keratic precipitates, and uveitis.

Illustrative Embodiment 97. The method of any one of Illustrative Embodiment 49-1 and Embodiments 49-2 to 73, wherein the method results in an efficacy result that is the subject has a CST of less than 320 μm or no more than 290 μm after administration of the surabgene lomparvovec.

Illustrative Embodiment 98. The method of Illustrative Embodiment 97, wherein the subject has a CST of less than 320 μm or no more than 290 μm at week 30 or 54 after administration of the surabgene lomparvovec.

Illustrative Embodiment 99. The method of Illustrative Embodiment 97 or 99, wherein the method results in a safety result that is mitigation of ocular inflammation of episcleritis, anterior chamber cell, vitritis, iridocyclitis, iritis, eye inflammation, anterior chamber flare, vitreal cells, keratic precipitates, and uveitis.

Illustrative Embodiment 100. The method of any one of Illustrative Embodiment 49-1 and Embodiments 49-2 to 73, wherein the method results in an efficacy result that is the subject not showing a worsening in their diabetic retinopathy severity scale (DRSS) score of two steps or more relative to baseline.

Illustrative Embodiment 101. The metho of Illustrative Embodiment 100, wherein the subject does not have a two or more step worsening in their ETDRS DRSS score at week 48, 96, or 144 after administration of the surabgene lomparvovec compared to their ETDRS DRSS score when the surabgene lomparvovec was administered.

Illustrative Embodiment 102. The method of Illustrative Embodiment 100 or 101, wherein the method results in a safety result that is mitigation of ocular inflammation of episcleritis, anterior chamber cell, vitritis, iridocyclitis, iritis, eye inflammation, anterior chamber flare, vitreal cells, keratic precipitates, and uveitis.

Illustrative Embodiment 103. The method of any one of Illustrative Embodiment 49-1 and Embodiments 49-2 to 73, wherein the method results in an efficacy result that is the subject not developing a vision-threatening complication (VTC) due to diabetic retinopathy.

Illustrative Embodiment 104. The method of Illustrative Embodiment 103, wherein the subject does not develop a vision-threatening complication (VTC) due to diabetic retinopathy by week 48, 96, or 144 after administration of the surabgene lomparvovec.

Illustrative Embodiment 105. The method of Illustrative Embodiment 103 or 104, wherein the VTC is CI-DME.

Illustrative Embodiment 106. The method of Illustrative Embodiment 103 or 104, wherein the VTC is proliferative diabetic retinopathy (PDR), wherein the PDR is characterized by one or more of (1) DRSS level increasing to 61 or higher, (2) presence of iris neovascularization of at least 2 cumulative clock hours and/or ANV on gonioscopy, and (2) presence of neovascular glaucoma.

Illustrative Embodiment 107. The method of any one of Illustrative Embodiments 103-104, wherein the method results in a safety result that is mitigation of ocular inflammation of episcleritis, anterior chamber cell, vitritis, iridocyclitis, iritis, eye inflammation, anterior chamber flare, vitreal cells, keratic precipitates, and uveitis.

Illustrative Embodiment 108. The method of any one of Illustrative Embodiment 49-1 and Embodiments 49-2 to 73, wherein the method results in an efficacy result that is the subject having either a one-step reduction in their DRSS score, no change in their DRSS score, or a one-step improvement in their ETDRS DRSS score.

Illustrative Embodiment 109. The method of Illustrative Embodiment 108, wherein the subject has either a one-step reduction in their ETDRS DRSS score, no change in their ETDRS DRSS score, or a one-step improvement in their ETDRS DRSS score at week 48, 96, or 144 after administration of the surabgene lomparvovec when compared to their ETDRS DRSS score when the surabgene lomparvovec was administered.

Illustrative Embodiment 110. The method Illustrative Embodiment 108 or 109, wherein the method results in a safety result that is mitigation of ocular inflammation of episcleritis, anterior chamber cell, vitritis, iridocyclitis, iritis, eye inflammation, anterior chamber flare, vitreal cells, keratic precipitates, and uveitis.

Illustrative Embodiment 111. The method of any one of Illustrative Embodiment 49-1 and Embodiments 49-2 to 73, wherein the method results in an efficacy result that is the subject maintains a ETDRS BCVA of within ±5 letters at week 48 after administration of the surabgene lomparvovec when compared to their ETDRS BCVA when the surabgene lomparvovec was administered.

Illustrative Embodiment 112. The method of Illustrative Embodiment 111, wherein the subject maintains a ETDRS BCVA of within ±5 letters at week 48, 96, or 104 after administration of the surabgene lomparvovec when compared to their ETDRS BCVA when the surabgene lomparvovec was administered.

Illustrative Embodiment 113. The method Illustrative Embodiment 111 or 112, wherein the method results in a safety result that is mitigation of ocular inflammation of episcleritis, anterior chamber cell, vitritis, iridocyclitis, iritis, eye inflammation, anterior chamber flare, vitreal cells, keratic precipitates, and uveitis.

Illustrative Embodiment 114. The method of any one of Illustrative Embodiment 49-1 and Embodiments 49-2 to 73, wherein the method results in an efficacy result that is the subject gains more than 5 letters of ETDRS BCVA at week 48 after administration of the surabgene lomparvovec when compared to their ETDRS BCVA when the surabgene lomparvovec was administered.

Illustrative Embodiment 115. The method of Illustrative Embodiment 114, wherein the subject gains more than 5 letters of ETDRS BCVA at week 48 after administration of the surabgene lomparvovec when compared to their ETDRS BCVA when the surabgene lomparvovec was administered.

Illustrative Embodiment 116. The method of any one of Illustrative Embodiment 49-1 and Embodiments 49-2 to 115, wherein therapeutically effective amount of surabgene lomparvovec is selected from the group consisting of 1×10¹² GC/eye, 1.5×10¹² GC/eye, and 2×10¹² GC/eye.

Illustrative Embodiment 117. The method of any one of Illustrative Embodiment 49-1 and Embodiments 49-2 to 116, wherein the subject has phakic lenses.

Illustrative Embodiment 118. The method of any one of Illustrative Embodiment 49-1 and Embodiments 49-2 to 116, wherein the subject has pseudophakic lenses.

Illustrative Embodiment 119. A method of treating a retinal disease associated with hVEGF in a subject in need thereof, comprising:
administering surabgene lomparvovec to the suprachoroidal space of an eye of said subject, wherein said administration of surabgene lomparvovec is at a dose in an amount of 1×10¹² GC/eye to 2×10¹² GC/eye; and
administering difluprednate or a pharmaceutically acceptable salt thereof to the eye of said subject prior to and after administration of surabgene lomparvovec;
wherein:
   the difluprednate or a pharmaceutically acceptable salt thereof is administered 1 drop 4 times daily for at least 3 days and up to 7 days prior to administration of the single dose of surabgene lomparvovec; and
   the dose of surabgene lomparvovec is safe and efficacious in treating said retinal disease at year 1 after administration of surabgene lomparvovec.

Illustrative Embodiment 120. The method of Illustrative Embodiment 119, wherein the dose of surabgene lomparvovec is safe and efficacious at year 2 after administration of surabgene lomparvovec.

Illustrative Embodiment 121. The method of Illustrative Embodiment 119 or 120, wherein the difluprednate or pharmaceutically acceptable salt thereof is administered at a dose of 0.05% (w/v).

Illustrative Embodiment 122. The method of Illustrative Embodiment 121, wherein the difluprednate or pharmaceutically acceptable salt thereof is a 0.05% (w/v) ophthalmic emulsion of difluprednate.

Illustrative Embodiment 123. The method of any one of Illustrative Embodiments 119-122, wherein the difluprednate or pharmaceutically acceptable salt thereof is administered topically.

Illustrative Embodiment 124. The method of any one of Illustrative Embodiments 119-123, wherein the difluprednate or pharmaceutically acceptable salt thereof is administered 1 drop 4 times daily for 7 days prior to administration of surabgene lomparvovec.

Illustrative Embodiment 125. The method of any one of Illustrative Embodiments 119-124, wherein administration of the difluprednate or pharmaceutically acceptable salt thereof continues for 7 to 16 weeks after said administration of surabgene lomparvovec.

Illustrative Embodiment 126. The method of any one of Illustrative Embodiments 119-124, wherein administration of the difluprednate or pharmaceutically acceptable salt thereof continues for 7 to 20 weeks after said administration of surabgene lomparvovec.

Illustrative Embodiment 127-1. The method of any one of Illustrative Embodiments 119-126 further comprising administering the difluprednate or a pharmaceutically acceptable salt thereof to the eye of said subject beginning Day 2 (the day immediately after the day of the administration of the dose of surabgene lomparvovec), 1 drop 4 times daily for 4 weeks, followed by 1 drop 3 times daily for 2 weeks, followed by 1 drop 2 times daily for 2 weeks, followed by 1 drop once daily for 2 weeks, and followed by 1 drop 1 time every other day for 2 weeks.

Illustrative Embodiment 127-2. The method of Illustrative Embodiment 127-1 further comprising administering 1 drop of the difluprednate or a pharmaceutically acceptable salt thereof to the eye of said subject during the day of, but after, the administration of the dose of surabgene lomparvovec.

Illustrative Embodiment 128-1. The method of any one of Illustrative Embodiments 119-126 further comprising administering the difluprednate or a pharmaceutically acceptable salt thereof to the eye of said subject beginning Day 2 (the day immediately after the day of the administration of the dose of surabgene lomparvovec), 1 drop 4 times daily for 5 weeks, followed by 1 drop 3 times daily for 3 weeks, followed by 1 drop 2 times daily for 3 weeks, followed by 1 drop once daily for 3 weeks, and followed by 1 drop 1 time every other day for 2 weeks.

Illustrative Embodiment 128-2. The method of Illustrative Embodiment 128-1 further comprising administering 1 drop of the difluprednate or a pharmaceutically acceptable salt thereof to the eye of said subject during the day of, but after, the administration of the dose of surabgene lomparvovec.

Illustrative Embodiment 129. A method of treating a retinal disease associated with hVEGF in a subject in need thereof, comprising:
administering surabgene lomparvovec to the suprachoroidal space of an eye of said subject, wherein said administration of surabgene lomparvovec is at a dose in an amount of 1×10¹² GC/eye to 2×10¹² GC/eye; and
administering prednisolone or a pharmaceutically acceptable salt thereof to the eye of said subject prior to and after administration of surabgene lomparvovec;
wherein:
   the prednisolone or a pharmaceutically acceptable salt thereof is administered 2 drops 6 times daily for at least 3 days and up to 7 days prior to administration of surabgene lomparvovec; and
   the surabgene lomparvovec is safe and efficacious in treating said retinal disease at year 1 after administration of the single dose of surabgene lomparvovec.

Illustrative Embodiment 130. The method of Illustrative Embodiment 129, wherein the dose of surabgene lomparvovec is safe and efficacious at year 2 after administration of surabgene lomparvovec.

Illustrative Embodiment 131. The method of Illustrative Embodiment 129 or 130, wherein the prednisolone or pharmaceutically acceptable salt thereof is administered at a dose of 1% (w/v).

Illustrative Embodiment 132. The method of Illustrative Embodiment 131, wherein the prednisolone or pharmaceutically acceptable salt thereof is prednisolone acetate.

Illustrative Embodiment 133. The method of Illustrative Embodiment 132, wherein the prednisolone acetate is a prednisolone acetate ophthalmic solution.

Illustrative Embodiment 134. The method of any one of Illustrative Embodiments 129-133, wherein the prednisolone or pharmaceutically acceptable salt thereof is administered topically.

Illustrative Embodiment 135. The method of any one of Illustrative Embodiments 129-134, wherein the prednisolone or pharmaceutically acceptable salt thereof is administered 2 drops 6 times daily for 7 days prior to administration of the surabgene lomparvovec.

Illustrative Embodiment 136. The method of any one of Illustrative Embodiments 129-135, wherein administration of the prednisolone or pharmaceutically acceptable salt thereof continues for 7 to 16 weeks after said administration of surabgene lomparvovec.

Illustrative Embodiment 137. The method of any one of Illustrative Embodiments 129-135, wherein administration of the prednisolone or pharmaceutically acceptable salt thereof continues for 7 to 20 weeks after said administration of surabgene lomparvovec.

Illustrative Embodiment 138-1. The method of any one of Illustrative Embodiments 129-137 further comprising administering the prednisolone or a pharmaceutically acceptable salt thereof to the eye of said subject beginning Day 2 (the day immediately after the day of the administration of the dose of surabgene lomparvovec), 2 drops 6 times daily for 4 weeks, followed by 2 drops 5 times daily for 1 week, followed by 2 drops 4 times daily for 1 week, followed by 2 drops 3 times daily for 1 week, followed by 2 drops 2 times daily for 1 week, followed by 2 drops once daily for 2 weeks, and followed by 2 drops 1 time every other day for 2 weeks.

Illustrative Embodiment 138-2. The method of Illustrative Embodiment 138-1 further comprising administering 2 drops of the prednisolone or a pharmaceutically acceptable salt thereof to the eye of said subject during the day of, but after, the administration of the dose of surabgene lomparvovec.

Illustrative Embodiment 139-1. The method of any one of Illustrative Embodiments 129-137 further comprising administering the prednisolone or a pharmaceutically acceptable salt thereof to the eye of said subject beginning Day 2 (the day immediately after the day of the administration of the dose of surabgene lomparvovec), 2 drops 6 times daily for 5 weeks, followed by 2 drops 5 times daily for 2 weeks, followed by 2 drops 4 times daily for 2 weeks, followed by 2 drops 3 times daily for 2 weeks, followed by 2 drops 2 times daily for 2 weeks, followed by 2 drops once daily for 1 week, and followed by 2 drops 1 time every other day for 2 weeks.

Illustrative Embodiment 139-2. The method of Illustrative Embodiment 139-1 further comprising administering 2 drops of the prednisolone or a pharmaceutically acceptable salt thereof to the eye of said subject during the day of, but after, the administration of the dose of surabgene lomparvovec.

Illustrative Embodiment 140. The method of any one of Illustrative Embodiments 129-139 further comprising waiting up to 5 minutes after administering a first drop of prednisolone or a pharmaceutically acceptable salt thereof to the eye of said subject to administer a second drop of prednisolone or a pharmaceutically acceptable salt thereof.

Illustrative Embodiment 141. The method of any one of Illustrative Embodiments 129-139 further comprising waiting at least 5 minutes to 10 minutes after administering a first drop of prednisolone or a pharmaceutically acceptable salt thereof to the eye of said subject to administer a second drop of prednisolone or a pharmaceutically acceptable salt thereof.

Illustrative Embodiment 142. The method of any one of Illustrative Embodiments 129-139 further comprising waiting 5 minutes after administering a first drop of prednisolone or a pharmaceutically acceptable salt thereof to the eye of said subject to administer a second drop of prednisolone or a pharmaceutically acceptable salt thereof.

Illustrative Embodiment 143. The method of any one of Illustrative Embodiments 119-142, wherein the retinal disease is nAMD.

Illustrative Embodiment 144-1. The method of any one of Illustrative Embodiments 119-142, wherein the retinal disease is DR.

Illustrative Embodiment 144-2. The method of Illustrative Embodiment 144-1, wherein the retinal disease is DR without DME.

Illustrative Embodiment 144-3. The method of Illustrative Embodiment 144-1, wherein the retinal disease is DR with DME.

Illustrative Embodiment 145. The method of any one of Illustrative Embodiments 119-142, wherein the retinal disease is DME.

Illustrative Embodiment 146. The method of any one of Illustrative Embodiments 119-145, wherein the dose of surabgene lomparvovec is selected from the group consisting of 1×10¹² GC/eye, 1.5×10¹² GC/eye, and 2×10¹² GC/eye.

Illustrative Embodiment 147. The method of any one of Illustrative Embodiments 119-146, wherein the dose of surabgene lomparvovec is 1×10¹² GC/eye.

Illustrative Embodiment 148. The method of any one of Illustrative Embodiments 119-146, wherein the dose of surabgene lomparvovec is 1.5×10¹² GC/eye.

Illustrative Embodiment 149. The method of any one of Illustrative Embodiments 119-146, wherein the dose of surabgene lomparvovec is 2×10¹² GC/eye.

Illustrative Embodiment 150. The method of Illustrative Embodiment 127-1 or 127-2, wherein the dose of surabgene lomparvovec is 1×10¹² GC/eye.

Illustrative Embodiment 151. The method of Illustrative Embodiment 128-1 or 128-2, wherein the dose of surabgene lomparvovec is 1.5×10¹² GC/eye.

Illustrative Embodiment 152. The method of any one of Illustrative Embodiments 138-1, 138-2, or 140-142, wherein the dose of surabgene lomparvovec is 1×10¹² GC/eye.

Illustrative Embodiment 153. The method of any one of Illustrative Embodiments 139-1, 139-2, or 140-142, wherein the dose of surabgene lomparvovec is 1.5×10¹² GC/eye.

Illustrative Embodiment 154. The method of any one of Illustrative Embodiments 119-153, wherein the efficacy result is selected from the group consisting of (1) the subject maintaining best-corrected visual acuity (BCVA) relative to baseline, (2) the subject showing an improvement in their diabetic retinopathy severity scale (DRSS) score relative to baseline, (3) the subject no longer having CI-DME that was present when the surabgene lomparvovec was administered, (4) the subject not developing any sight-threatening ocular diabetic complications in the eye treated with surabgene lomparvovec, (5) the subject not developing ocular diabetic complications requiring treatment per standard of care (SOC) for such complications in the eye treated with surabgene lomparvovec, (6) if the subject develops neovascularization due to diabetic retinopathy requiring treatment per standard of care (SOC), then the subject only requires one SOC treatment to treat the neovascularization, (7) the subject not developing ocular diabetic complications requiring surgical intervention per standard of care (SOC) for such complications in the eye treated with surabgene lomparvovec, (8) the subject has a CST of less than 320 μm or no more than 290 μm after administration of the surabgene lomparvovec, (9) the subject not showing a worsening in their diabetic retinopathy severity scale (DRSS) score of two steps or more relative to baseline, (10) the subject not developing a vision-threatening complication (VTC) due to diabetic retinopathy, (11) subject having either a one-step reduction in their DRSS score, no change in their DRSS score, or a one-step improvement in their ETDRS DRSS score, (12) the subject maintains a ETDRS BCVA of within ±5 letters at week 48 after administration of the surabgene lomparvovec when compared to their ETDRS BCVA when the surabgene lomparvovec was administered, (13) the subject gains more than 5 letters of ETDRS BCVA at week 48 after administration of the surabgene lomparvovec when compared to their ETDRS BCVA when the surabgene lomparvovec was administered; and/or
wherein the safety result is mitigation of ocular inflammation of episcleritis, anterior chamber cell, vitritis, iridocyclitis, iritis, eye inflammation, anterior chamber flare, vitreal cells, keratic precipitates, and uveitis.

Illustrative Embodiment 155. A method of treating a retinal disease associated with hVEGF in a subject in need thereof, comprising:
administering surabgene lomparvovec to the suprachoroidal space of an eye of said subject, wherein said administration of surabgene lomparvovec is at a dose in an amount of 1×10¹² GC/eye to 2×10¹² GC/eye; and
topically administering an anti-inflammatory steroid to the eye of said subject prior to and after said administration of surabgene lomparvovec.

Illustrative Embodiment 156. The method of Illustrative Embodiment 155, wherein the anti-inflammatory steroid is difluprednate or a pharmaceutically acceptable salt thereof, and is administered for 7 days prior to said administration of surabgene lomparvovec.

Illustrative Embodiment 157. The method of Illustrative Embodiment 155, where the anti-inflammatory steroid is difluprednate or a pharmaceutically acceptable salt thereof, and is administered 1 drop 4 times daily for at least 3 days and up to 7 days prior to said administration of surabgene lomparvovec.

Illustrative Embodiment 158. The method of Illustrative Embodiment 156 or 157, wherein the difluprednate or pharmaceutically acceptable salt thereof is administered at a dose of 0.05% (w/v).

Illustrative Embodiment 159. The method of Illustrative Embodiment 158, wherein the difluprednate or pharmaceutically acceptable salt thereof is a 0.05% (w/v) ophthalmic emulsion of difluprednate.

Illustrative Embodiment 160. The method of any one of Illustrative Embodiments 156-159, wherein the difluprednate or pharmaceutically acceptable salt thereof is administered 1 drop 4 times daily for 7 days prior to said administration of surabgene lomparvovec.

Illustrative Embodiment 161. The method of any one of Illustrative Embodiments 156-160, wherein administration of the difluprednate or pharmaceutically acceptable salt thereof continues for 7 to 16 weeks after said administration of surabgene lomparvovec.

Illustrative Embodiment 162. The method of Illustrative Embodiment 161, wherein administration of the difluprednate or pharmaceutically acceptable salt thereof continues for 12 weeks after said administration of surabgene lomparvovec.

Illustrative Embodiment 163. The method of any one of Illustrative Embodiments 156-162, further comprising administering the difluprednate or pharmaceutically acceptable salt thereof to the eye of said subject beginning Day 2 (the day immediately after the day of the administration of the dose of surabgene lomparvovec), 1 drop 4 times daily for 4 weeks, followed by 1 drop 3 times daily for 2 weeks, followed by 1 drop 2 times daily for 2 weeks, followed by 1 drop once daily for 2 weeks, and followed by 1 drop 1 time every other day for 2 weeks.

Illustrative Embodiment 164. The method of Illustrative Embodiment 163 further comprising administering 1 drop of the difluprednate or a pharmaceutically acceptable salt thereof to the eye of said subject during the day of, but after, the administration of the dose of surabgene lomparvovec.

Illustrative Embodiment 165. The method of any one of Illustrative Embodiments 156-161 further comprising administering the difluprednate or pharmaceutically acceptable salt thereof to the eye of said subject beginning Day 2 (the day immediately after the day of the administration of the dose of surabgene lomparvovec), 1 drop 4 times daily for 5 weeks, followed by 1 drop 3 times daily for 3 weeks, followed by 1 drop 2 times daily for 3 weeks, followed by 1 drop once daily for 3 weeks, and followed by 1 drop 1 time every other day for 2 weeks.

Illustrative Embodiment 166. The method of Illustrative Embodiment 165 further comprising administering 1 drop of the difluprednate or a pharmaceutically acceptable salt thereof to the eye of said subject during the day of, but after, the administration of the dose of surabgene lomparvovec.

Illustrative Embodiment 167. The method of any one of Illustrative Embodiments 155-166, wherein the dose of surabgene lomparvovec is safe and efficacious in treating said retinal disease at year 1 after said administration of surabgene lomparvovec.

Illustrative Embodiment 168. The method of any one of Illustrative Embodiments 155-166, wherein the dose of surabgene lomparvovec is safe and efficacious in treating said retinal disease at year 2 after said administration of surabgene lomparvovec.

Illustrative Embodiment 169. The method of Illustrative Embodiment 155, wherein the anti-inflammatory steroid is prednisolone or a pharmaceutically acceptable salt thereof, and is administered 7 days prior to said administration of surabgene lomparvovec.

Illustrative Embodiment 170. The method of Illustrative Embodiment 155, wherein the anti-inflammatory steroid is prednisolone or a pharmaceutically acceptable salt thereof, and is administered 2 drops 6 times daily for at least 3 days and up to 7 days prior to said administration of surabgene lomparvovec.

Illustrative Embodiment 171. The method of Illustrative Embodiment 169 or 170, wherein the anti-inflammatory steroid is prednisolone acetate.

Illustrative Embodiment 172. The method of Illustrative Embodiment 171, wherein the prednisolone acetate is administered at a dose of 1% (w/v).

Illustrative Embodiment 173. The method of Illustrative Embodiment 172, wherein the prednisolone acetate is a prednisolone acetate ophthalmic suspension.

Illustrative Embodiment 174. The method of any one of Illustrative Embodiments 169-173, wherein the prednisolone or pharmaceutically acceptable salt thereof is administered 2 drops 6 times daily for 7 days prior to said administration of surabgene lomparvovec.

Illustrative Embodiment 175. The method of any one of Illustrative Embodiments 169-174, wherein administration of the prednisolone or pharmaceutically acceptable salt thereof continues for 7 to 16 weeks after said administration of surabgene lomparvovec.

Illustrative Embodiment 176. The method of Illustrative Embodiment 175, wherein administration of the prednisolone or pharmaceutically acceptable salt thereof continues for 12 weeks after said administration of surabgene lomparvovec.

Illustrative Embodiment 177. The method of any one of Illustrative Embodiments 169-176 further comprising administering the prednisolone or pharmaceutically acceptable salt thereof to the eye of said subject beginning Day 2 (the day immediately after the day of the administration of the dose of surabgene lomparvovec), 2 drops 6 times daily for 4 weeks, followed by 2 drops 5 times daily for 1 week, followed by 2 drops 4 times daily for 1 week, followed by 2 drops 3 times daily for 1 week, followed by 2 drops 2 times daily for 1 week, followed by 2 drops once daily for 2 weeks, and followed by 2 drops 1 time every other day for 2 weeks.

Illustrative Embodiment 178. The method of Illustrative Embodiment 177 further comprising administering 2 drops of the prednisolone or pharmaceutically acceptable salt thereof to the eye of said subject during the day of, but after, the administration of the dose of surabgene lomparvovec.

Illustrative Embodiment 179. The method of any one of Illustrative Embodiments 169-175, further comprising administering the prednisolone or pharmaceutically acceptable salt thereof to the eye of said subject beginning Day 2 (the day immediately after the day of the administration of the dose of surabgene lomparvovec), 2 drops 6 times daily for 5 weeks, followed by 2 drops 5 times daily for 2 weeks, followed by 2 drops 4 times daily for 2 weeks, followed by 2 drops 3 times daily for 2 weeks, followed by 2 drops 2 times daily for 2 weeks, followed by 2 drops once daily for 1 week, and followed by 2 drops 1 time every other day for 2 weeks.

Illustrative Embodiment 180. The method of Illustrative Embodiment 179 further comprising administering 2 drops of the prednisolone or a pharmaceutically acceptable salt thereof to the eye of said subject during the day of, but after, the administration of the dose of surabgene lomparvovec.

Illustrative Embodiment 181. The method of any one of Illustrative Embodiments 169-180, wherein the dose of surabgene lomparvovec is safe and efficacious in treating said retinal disease at year 1 after said administration of surabgene lomparvovec.

Illustrative Embodiment 182. The method of any one of Illustrative Embodiments 169-180, wherein the dose of surabgene lomparvovec is safe and efficacious in treating said retinal disease at year 2 after said administration of surabgene lomparvovec.

Illustrative Embodiment 183. The method of any one of Illustrative Embodiments 170-182, further comprising waiting up to 5 minutes after administering a first drop of the prednisolone or pharmaceutically acceptable salt thereof to the eye of said subject to administer a second drop of the prednisolone or pharmaceutically acceptable salt thereof.

Illustrative Embodiment 184. The method of any one of Illustrative Embodiments 170-182, further comprising waiting at least 5 minutes to 10 minutes after administering a first drop of the prednisolone or pharmaceutically acceptable salt thereof to the eye of said subject to administer a second drop of the prednisolone or pharmaceutically acceptable salt thereof.

Illustrative Embodiment 185. The method of any one of Illustrative Embodiments 170-182 further comprising waiting 5 minutes after administering a first drop of prednisolone or a pharmaceutically acceptable salt thereof to the eye of said subject to administer a second drop of prednisolone or a pharmaceutically acceptable salt thereof.

Illustrative Embodiment 186. The method of any one of Illustrative Embodiments 155-185, wherein the retinal disease is nAMD.

Illustrative Embodiment 187. The method of any one of Illustrative Embodiments 155-185, wherein the retinal disease is DR.

Illustrative Embodiment 188. The method of any one of Illustrative Embodiments 155-185, wherein the retinal disease is DME.

Illustrative Embodiment 189. The method of Illustrative Embodiment 187, wherein the retinal disease is DR without DME.

Illustrative Embodiment 190. The method of Illustrative Embodiment 187, wherein the retinal disease is DR with DME.

Illustrative Embodiment 191. The method of any one of Illustrative Embodiments 155-190, wherein the dose of surabgene lomparvovec is selected from the group consisting of 1×10¹² GC/eye, 1.5×10¹² GC/eye, and 2×10¹² GC/eye.

Illustrative Embodiment 192. The method of any one of Illustrative Embodiments 155-191, wherein the dose of surabgene lomparvovec is 1×10¹² GC/eye.

Illustrative Embodiment 193. The method of any one of Illustrative Embodiments 155-191, wherein the dose of surabgene lomparvovec is 1.5×10¹² GC/eye.

Illustrative Embodiment 194. The method of any one of Illustrative Embodiments 155-191, wherein the dose of surabgene lomparvovec is 2×10¹² GC/eye.

Illustrative Embodiment 195. A method of treating a retinal disease associated with hVEGF in a subject in need thereof, comprising:
administering surabgene lomparvovec to the suprachoroidal space of an eye of said subject, wherein said administration of surabgene lomparvovec is at a dose in an amount of 1×10¹² GC/eye to 2×10¹² GC/eye; and
topically administering an anti-inflammatory steroid to the eye of said subject prior to and after said administration of surabgene lomparvovec.

Illustrative Embodiment 196. The method of Illustrative Embodiment 195, wherein the anti-inflammatory steroid is administered daily for 7 days prior to said administration of surabgene lomparvovec, followed by a tapering dose schedule during 12 to 27 weeks, including 12 weeks, 16 weeks, 20 weeks, 21 weeks, 22 weeks, 23 weeks, 24 weeks, 25 weeks, 26 weeks, or 27 weeks, after said administration of surabgene lomparvovec.

Illustrative Embodiment 197. The method of Illustrative Embodiment 196, wherein said retinal disease is nAMD, DR or DME.

Illustrative Embodiment 198. The method of Illustrative Embodiment 197, wherein the dose of surabgene lomparvovec is 1×10¹² GC/eye, and is safe and efficacious in treating said retinal disease at year 1 after said administration of surabgene lomparvovec without additional administration of surabgene lomparvovec or any anti-hVEGF agent.

Illustrative Embodiment 199. A method of treating a retinal disease associated with hVEGF in a subject in need thereof, comprising:
administering surabgene lomparvovec to the suprachoroidal space of an eye of said subject, wherein said administration of surabgene lomparvovec is at a dose in an amount of 1×10¹² GC/eye to 2×10¹² GC/eye; and
topically administering difluprednate or a pharmaceutically acceptable salt thereof to the eye of said subject prior to and after said administration of surabgene lomparvovec.

Illustrative Embodiment 200. The method of Illustrative Embodiment 199, wherein the difluprednate or pharmaceutically acceptable salt thereof is administered daily for 7 days prior to said administration of surabgene lomparvovec, followed by a tapering dose schedule during 12 to 27 weeks, including 12 weeks, 16 weeks, 20 weeks, 21 weeks, 22 weeks, 23 weeks, 24 weeks, 25 weeks, 26 weeks, or 27 weeks, after said administration of surabgene lomparvovec.

Illustrative Embodiment 201. The method of Illustrative Embodiment 200, wherein the difluprednate or pharmaceutically acceptable salt thereof is administered 1 drop 4 times daily for 7 days prior to said administration of surabgene lomparvovec.

Illustrative Embodiment 202. The method of Illustrative Embodiment 201, wherein the tapering dose schedule comprises administering the difluprednate or pharmaceutically acceptable salt thereof to the eye of said subject beginning Day 2 (the day immediately after the day of the administration of the dose of surabgene lomparvovec), 1 drop 4 times daily for 4 weeks, followed by 1 drop 3 times daily for 2 weeks, followed by 1 drop 2 times daily for 2 weeks, followed by 1 drop once daily for 2 weeks, and followed by 1 drop 1 time every other day for 2 weeks.

Illustrative Embodiment 203. The method of Illustrative Embodiment 202, further comprising administering 1 drop of the difluprednate or pharmaceutically acceptable salt thereof to the eye of said subject during the day of, but after, the administration of the dose of surabgene lomparvovec.

Illustrative Embodiment 204. The method of Illustrative Embodiment 200, wherein the retinal disease is nAMD, DR or DME.

Illustrative Embodiment 205. The method of Illustrative Embodiment 204, wherein the dose of surabgene lomparvovec is 1×10¹² GC/eye.

Illustrative Embodiment 206. The method of Illustrative Embodiment 205, wherein the dose of surabgene lomparvovec is safe and efficacious in treating said retinal disease at year 1 after said administration of surabgene lomparvovec without additional administration of surabgene lomparvovec or any anti-hVEGF agent.

Illustrative Embodiment 207. A method of treating a retinal disease associated with hVEGF in a subject in need thereof, comprising:
administering surabgene lomparvovec to the suprachoroidal space of an eye of said subject, wherein said administration of surabgene lomparvovec is at a dose in an amount of 1×10¹² GC/eye to 2×10¹² GC/eye; and
topically administering prednisolone or a pharmaceutically acceptable salt thereof to the eye of said subject prior to and after said administration of surabgene lomparvovec.

Illustrative Embodiment 208. The method of Illustrative Embodiment 207, wherein the prednisolone or pharmaceutically acceptable salt thereof is administered daily for 7 days prior to said administration of surabgene lomparvovec, followed by a tapering dose schedule during 12 to 27 weeks, including 12 weeks, 16 weeks, 20 weeks, 21 weeks, 22 weeks, 23 weeks, 24 weeks, 25 weeks, 26 weeks, or 27 weeks, after said administration of surabgene lomparvovec.

Illustrative Embodiment 209. The method of Illustrative Embodiment 208, wherein the prednisolone or pharmaceutically acceptable salt thereof is administered 2 drops 6 times daily for 7 days prior to said administration of surabgene lomparvovec.

Illustrative Embodiment 210. The method of Illustrative Embodiment 209, wherein the tapering dose schedule comprises administering the prednisolone or pharmaceutically acceptable salt thereof to the eye of said subject beginning Day 2 (the day immediately after the day of the administration of the dose of surabgene lomparvovec), 2 drops 6 times daily for 4 weeks, followed by 2 drops 5 times daily for 1 week, followed by 2 drops 4 times daily for 1 week, followed by 2 drops 3 times daily for 1 week, followed by 2 drops 2 times daily for 1 week, followed by 2 drops once daily for 2 weeks, and followed by 2 drops 1 time every other day for 2 weeks.

Illustrative Embodiment 211. The method of Illustrative Embodiment 210, further comprising administering 2 drops of the prednisolone or pharmaceutically acceptable salt thereof to the eye of said subject during the day of, but after, the administration of the dose of surabgene lomparvovec.

Illustrative Embodiment 212. The method of Illustrative Embodiment 208, wherein the retinal disease is nAMD, DR or DME.

Illustrative Embodiment 213. The method of Illustrative Embodiment 212, wherein the dose of surabgene lomparvovec is 1×10¹² GC/eye.

Illustrative Embodiment 214. The method of Illustrative Embodiment 213, wherein the dose of surabgene lomparvovec is safe and efficacious in treating said retinal disease at year 1 after said administration of surabgene lomparvovec without additional administration of surabgene lomparvovec or any anti-hVEGF agent.

What is claimed is:

1. A method of treating a retinal disease associated with hVEGF in a subject in need thereof, comprising:
administering surabgene lomparvovec to the suprachoroidal space of an eye of said subject, wherein said administration of surabgene lomparvovec is at a dose in an amount of 1×10¹² GC/eye to 2×10¹² GC/eye; and
topically administering an anti-inflammatory steroid to the eye of said subject prior to and after said administration of surabgene lomparvovec.

2. The method of claim 1, wherein the anti-inflammatory steroid is administered daily for 7 days prior to said administration of surabgene lomparvovec, followed by a tapering dose schedule during 12 to 27 weeks after said administration of surabgene lomparvovec.

3. The method of claim 2, wherein said retinal disease is nAMD, DR or DME.

4. The method of claim 3, wherein the dose of surabgene lomparvovec is 1×10¹² GC/eye, and is safe and efficacious in treating said retinal disease at year 1 after said administration of surabgene lomparvovec without additional administration of surabgene lomparvovec or any anti-hVEGF agent.

5. A method of treating a retinal disease associated with hVEGF in a subject in need thereof, comprising:
administering surabgene lomparvovec to the suprachoroidal space of an eye of said subject, wherein said administration of surabgene lomparvovec is at a dose in an amount of 1×10¹² GC/eye to 2×10¹² GC/eye; and
topically administering difluprednate or a pharmaceutically acceptable salt thereof to the eye of said subject prior to and after said administration of surabgene lomparvovec.

6. The method of claim 5, wherein the difluprednate or pharmaceutically acceptable salt thereof is administered daily for 7 days prior to said administration of surabgene lomparvovec, followed by a tapering dose schedule during 12 to 27 weeks after said administration of surabgene lomparvovec.

7. The method of claim 6, wherein the difluprednate or pharmaceutically acceptable salt thereof is administered 1 drop 4 times daily for 7 days prior to said administration of surabgene lomparvovec.

8. The method of claim 7, wherein the tapering dose schedule comprises administering the difluprednate or pharmaceutically acceptable salt thereof to the eye of said subject beginning Day 2 (the day immediately after the day of the administration of the dose of surabgene lomparvovec), 1 drop 4 times daily for 4 weeks, followed by 1 drop 3 times daily for 2 weeks, followed by 1 drop 2 times daily for 2 weeks, followed by 1 drop once daily for 2 weeks, and followed by 1 drop 1 time every other day for 2 weeks.

9. The method of claim 8, further comprising administering 1 drop of the difluprednate or pharmaceutically acceptable salt thereof to the eye of said subject during the day of, but after, the administration of the dose of surabgene lomparvovec.

10. The method of claim 6, wherein the retinal disease is nAMD, DR or DME.

11. The method of claim 10, wherein the dose of surabgene lomparvovec is 1×10¹² GC/eye.

12. The method of claim 11, wherein the dose of surabgene lomparvovec is safe and efficacious in treating said retinal disease at year 1 after said administration of surabgene lomparvovec without additional administration of surabgene lomparvovec or any anti-hVEGF agent.

13. A method of treating a retinal disease associated with hVEGF in a subject in need thereof, comprising:
administering surabgene lomparvovec to the suprachoroidal space of an eye of said subject, wherein said administration of surabgene lomparvovec is at a dose in an amount of 1×10¹² GC/eye to 2×10¹² GC/eye; and
topically administering prednisolone or a pharmaceutically acceptable salt thereof to the eye of said subject prior to and after said administration of surabgene lomparvovec.

14. The method of claim 13, wherein the prednisolone or pharmaceutically acceptable salt thereof is administered daily for 7 days prior to said administration of surabgene lomparvovec, followed by a tapering dose schedule during 12 to 27 weeks after said administration of surabgene lomparvovec.

15. The method of claim 14, wherein the prednisolone or pharmaceutically acceptable salt thereof is administered 2 drops 6 times daily for 7 days prior to said administration of surabgene lomparvovec.

16. The method of claim 15, wherein the tapering dose schedule comprises administering the prednisolone or pharmaceutically acceptable salt thereof to the eye of said subject beginning Day 2 (the day immediately after the day of the administration of the dose of surabgene lomparvovec), 2 drops 6 times daily for 4 weeks, followed by 2 drops 5 times daily for 1 week, followed by 2 drops 4 times daily for 1 week, followed by 2 drops 3 times daily for 1 week, followed by 2 drops 2 times daily for 1 week, followed by 2 drops once daily for 2 weeks, and followed by 2 drops 1 time every other day for 2 weeks.

17. The method of claim 16, further comprising administering 2 drops of the prednisolone or pharmaceutically acceptable salt thereof to the eye of said subject during the day of, but after, the administration of the dose of surabgene lomparvovec.

18. The method of claim 14, wherein the retinal disease is nAMD, DR or DME.

19. The method of claim 18, wherein the dose of surabgene lomparvovec is 1×10¹² GC/eye.

20. The method of claim 19, wherein the dose of surabgene lomparvovec is safe and efficacious in treating said retinal disease at year 1 after said administration of surabgene lomparvovec without additional administration of surabgene lomparvovec or any anti-hVEGF agent.
